(19) United States
(12) Patent Application Publication
(10) Pub. No.: US 2024/0079130 A1
(43) Pub. Date: Mar. 7, 2024

(54) USER INTERFACES FOR HEALTH TRACKING

(71) Applicant: Apple Inc., Cupertino, CA (US)

(72) Inventors: Lindsey MARATTA, Sunnyvale, CA (US); Roxanne B. BRITTAIN, San Francisco, CA (US); Nicholas D. FELTON, Sunnyvale, CA (US); Karen L. JENKINS, Mountain View, CA (US); Michael PANGBURN, San Jose, CA (US)

(21) Appl. No.: 18/237,339

(22) Filed: Aug. 23, 2023

Related U.S. Application Data
(60) Provisional application No. 63/404,139, filed on Sep. 6, 2022.

(51) Int. Cl. G16H 40/67; A61B 5/00; G16H 50/30

(52) U.S. Cl. CPC G16H 40/67; A61B 5/7475; G16H 50/30

(57) ABSTRACT

The present disclosure generally relates to tracking health information and generating related predictions and/or notifications.

### CROSS-REFERENCE TO RELATED APPLICATIONS

This application claims priority to U.S. Provisional Patent Application No. 63/404,139, filed Sep. 6, 2022, the contents of which are incorporated by reference in their entirety.

### FIELD

The present disclosure relates generally to computer user interfaces, and more specifically to techniques for tracking health information and generating predictions and notifications.

### BACKGROUND

Recurring (e.g., reoccurring) events, such as recurring health events can be tracked on electronic devices to log past events and to predict future events.

### BRIEF SUMMARY

Some techniques for tracking health information using electronic devices, however, are generally cumbersome and inefficient. For example, some existing techniques use a complex and time-consuming user interface, which may include multiple key presses or keystrokes. Existing techniques require more time than necessary, wasting user time and device energy. This latter consideration is particularly important in battery-operated devices.

Accordingly, the present technique provides electronic devices with faster, more efficient methods and interfaces for tracking health information and for generating related predictions and/or notifications. Such methods and interfaces optionally complement or replace other methods for tracking health information and for generating related predictions and/or notifications. Such methods and interfaces reduce the cognitive burden on a user and produce a more efficient human-machine interface. For battery-operated computing devices, such methods and interfaces conserve power and increase the time between battery charges.

In accordance with some embodiments, a method is described. The method comprises, at a computer system that is in communication with a display generation component, one or more input devices, and a temperature sensor: receiving, via the one or more input devices, an input corresponding to a request to enter a sleep tracking mode; in response to the input, entering the sleep tracking mode; during the sleep tracking mode, causing a first set of user body temperature information to be collected via the temperature sensor; receiving notification data of a first type; in response to receiving the notification data of the first type: in accordance with a determination that the computer system is not in the sleep tracking mode, outputting a first notification corresponding to the notification data of the first type; and in accordance with a determination that the computer system is currently in the sleep tracking mode, forgoing outputting the first notification corresponding to the notification data of the first type; and after causing the first set of user body temperature information to be collected, displaying, via display generation component, a body temperature user interface that includes representations of one or more sets of user body temperature information including the first set of user body temperature information.

In accordance with some embodiments, a non-transitory computer-readable storage medium is described. The non-transitory computer-readable storage medium stores one or more programs configured to be executed by one or more processors of a computer system that is in communication with a display generation component, one or more input devices, and a temperature sensor, the one or more programs including instructions for: receiving, via the one or more input devices, an input corresponding to a request to enter a sleep tracking mode; in response to the input, entering the sleep tracking mode; during the sleep tracking mode, causing a first set of user body temperature information to be collected via the temperature sensor; receiving notification data of a first type; in response to receiving the notification data of the first type: in accordance with a determination that the computer system is not in the sleep tracking mode, outputting a first notification corresponding to the notification data of the first type; and in accordance with a determination that the computer system is currently in the sleep tracking mode, forgoing outputting the first notification corresponding to the notification data of the first type; and after causing the first set of user body temperature information to be collected, displaying, via display generation component, a body temperature user interface that includes representations of one or more sets of user body temperature information including the first set of user body temperature information.

In accordance with some embodiments, a transitory computer-readable storage medium is described. The transitory computer-readable storage medium stores one or more programs configured to be executed by one or more processors of a computer system that is in communication with a display generation component, one or more input devices, and a temperature sensor, the one or more programs including instructions for: receiving, via the one or more input devices, an input corresponding to a request to enter a sleep tracking mode; in response to the input, entering the sleep tracking mode; during the sleep tracking mode, causing a first set of user body temperature information to be collected via the temperature sensor; receiving notification data of a first type; in response to receiving the notification data of the first type: in accordance with a determination that the computer system is not in the sleep tracking mode, outputting a first notification corresponding to the notification data of the first type; and in accordance with a determination that the computer system is currently in the sleep tracking mode, forgoing outputting the first notification corresponding to the notification data of the first type; and after causing the first set of user body temperature information to be collected, displaying, via display generation component, a body temperature user interface that includes representations of one or more sets of user body temperature information including the first set of user body temperature information.

In accordance with some embodiments, a computer system is described. The computer system is configured to communicate with a display generation component, one or more input devices, and a temperature sensor, and comprises: one or more processors; and memory storing one or more programs configured to be executed by the one or more processors, the one or more programs including instructions for: receiving, via the one or more input devices, an input corresponding to a request to enter a sleep tracking mode; in response to the input, entering the sleep tracking mode; during the sleep tracking mode, causing a first set of user body temperature information to be collected via the temperature sensor; receiving notification data of a first type; in response to receiving the notification data of the first type: in accordance with a determination that the computer system is not in the sleep tracking mode, outputting a first notification corresponding to the notification data of the first type; and in accordance with a determination that the computer system is currently in the sleep tracking mode, forgoing outputting the first notification corresponding to the notification data of the first type; and after causing the first set of user body temperature information to be collected, displaying, via display generation component, a body temperature user interface that includes representations of one or more sets of user body temperature information including the first set of user body temperature information.

In accordance with some embodiments, a computer system is described. The computer system is configured to communicate with a display generation component, one or more input devices, and a temperature sensor, and comprises: means for receiving, via the one or more input devices, an input corresponding to a request to enter a sleep tracking mode; in response to the input, entering the sleep tracking mode; means for, during the sleep tracking mode, causing a first set of user body temperature information to be collected via the temperature sensor; receiving notification data of a first type; means for, in response to receiving the notification data of the first type: in accordance with a determination that the computer system is not in the sleep tracking mode, outputting a first notification corresponding to the notification data of the first type; and in accordance with a determination that the computer system is currently in the sleep tracking mode, forgoing outputting the first notification corresponding to the notification data of the first type; and means for, after causing the first set of user body temperature information to be collected, displaying, via display generation component, a body temperature user interface that includes representations of one or more sets of user body temperature information including the first set of user body temperature information.

In accordance with some embodiments, a computer program product is described. The computer program product comprises one or more programs configured to be executed by one or more processors of a computer system that is in communication with a display generation component, one or more input devices, and a temperature sensor, the one or more programs including instructions for: receiving, via the one or more input devices, an input corresponding to a request to enter a sleep tracking mode; in response to the input, entering the sleep tracking mode; during the sleep tracking mode, causing a first set of user body temperature information to be collected via the temperature sensor; receiving notification data of a first type; in response to receiving the notification data of the first type: in accordance with a determination that the computer system is not in the sleep tracking mode, outputting a first notification corresponding to the notification data of the first type; and in accordance with a determination that the computer system is currently in the sleep tracking mode, forgoing outputting the first notification corresponding to the notification data of the first type; and after causing the first set of user body temperature information to be collected, displaying, via display generation component, a body temperature user interface that includes representations of one or more sets of user body temperature information including the first set of user body temperature information.

In accordance with some embodiments, a method is described. The method comprises, at a computer system that is in communication with a display generation component and one or more input devices: receiving body temperature information corresponding to a user; and after receiving the body temperature information corresponding to the user, displaying, via the display generation component, a push notification that causes the display generation component to transition from an inactive state to an active state and that indicates to the user that a predicted ovulation date has been determined for the user, wherein the predicted ovulation date was determined based on the body temperature information corresponding to the user.

In accordance with some embodiments, a non-transitory computer-readable storage medium is described. The non-transitory computer-readable storage medium stores one or more programs configured to be executed by one or more processors of a computer system that is in communication with a display generation component and one or more input devices, the one or more programs including instructions for: receiving body temperature information corresponding to a user; and after receiving the body temperature information corresponding to the user, displaying, via the display generation component, a push notification that causes the display generation component to transition from an inactive state to an active state and that indicates to the user that a predicted ovulation date has been determined for the user, wherein the predicted ovulation date was determined based on the body temperature information corresponding to the user.

In accordance with some embodiments, a transitory computer-readable storage medium is described. The transitory computer-readable storage medium stores one or more programs configured to be executed by one or more processors of a computer system that is in communication with a display generation component and one or more input devices, the one or more programs including instructions for: receiving body temperature information corresponding to a user; and after receiving the body temperature information corresponding to the user, displaying, via the display generation component, a push notification that causes the display generation component to transition from an inactive state to an active state and that indicates to the user that a predicted ovulation date has been determined for the user, wherein the predicted ovulation date was determined based on the body temperature information corresponding to the user.

In accordance with some embodiments, a computer system is described. The computer system is configured to communicate with a display generation component and one or more input devices, and comprises: one or more processors; and memory storing one or more programs configured to be executed by the one or more processors, the one or more programs including instructions for: receiving body temperature information corresponding to a user; and after receiving the body temperature information corresponding to the user, displaying, via the display generation component, a push notification that causes the display generation component to transition from an inactive state to an active state and that indicates to the user that a predicted ovulation date has been determined for the user, wherein the predicted ovulation date was determined based on the body temperature information corresponding to the user.

In accordance with some embodiments, a computer system is described. The computer system is configured to communicate with a display generation component and one or more input devices. The computer system comprises: means for receiving body temperature information corresponding to a user; and means for, after receiving the body temperature information corresponding to the user, displaying, via the display generation component, a push notification that causes the display generation component to transition from an inactive state to an active state and that indicates to the user that a predicted ovulation date has been determined for the user, wherein the predicted ovulation date was determined based on the body temperature information corresponding to the user.

In accordance with some embodiments, a computer program product is described. The computer program product comprises one or more programs configured to be executed by one or more processors of a computer system that is in communication with a display generation component and one or more input devices, the one or more programs including instructions for: receiving body temperature information corresponding to a user; and after receiving the body temperature information corresponding to the user, displaying, via the display generation component, a push notification that causes the display generation component to transition from an inactive state to an active state and that indicates to the user that a predicted ovulation date has been determined for the user, wherein the predicted ovulation date was determined based on the body temperature information corresponding to the user.

In accordance with some embodiments, a method is described. The method comprises, at a computer system that is in communication with a display generation component and one or more input devices: displaying, via the display generation component, at a first time, a cycle tracking user interface including displaying a first fertile window prediction for a user; receiving body temperature information corresponding to the user; after receiving the body temperature information corresponding to the user, determining, at a second time subsequent to the first time, based on the body temperature information corresponding to the user, a predicted ovulation date for the user; and displaying, via the display generation component, at a third time subsequent to the second time, an updated cycle tracking user interface, including concurrently displaying: a representation of the predicted ovulation date for the user; and an updated fertile window prediction for the user different from the first fertile window prediction for the user.

In accordance with some embodiments, a non-transitory computer-readable storage medium is described. The non-transitory computer-readable storage medium stores one or more programs configured to be executed by one or more processors of a computer system that is in communication with a display generation component and one or more input devices, the one or more programs including instructions for: displaying, via the display generation component, at a first time, a cycle tracking user interface including displaying a first fertile window prediction for a user; receiving body temperature information corresponding to the user; after receiving the body temperature information corresponding to the user, determining, at a second time subsequent to the first time, based on the body temperature information corresponding to the user, a predicted ovulation date for the user; and displaying, via the display generation component, at a third time subsequent to the second time, an updated cycle tracking user interface, including concurrently displaying: a representation of the predicted ovulation date for the user; and an updated fertile window prediction for the user different from the first fertile window prediction for the user.

In accordance with some embodiments, a transitory computer-readable storage medium is described. The transitory computer-readable storage medium stores one or more programs configured to be executed by one or more processors of a computer system that is in communication with a display generation component and one or more input devices, the one or more programs including instructions for: displaying, via the display generation component, at a first time, a cycle tracking user interface including displaying a first fertile window prediction for a user; receiving body temperature information corresponding to the user; after receiving the body temperature information corresponding to the user, determining, at a second time subsequent to the first time, based on the body temperature information corresponding to the user, a predicted ovulation date for the user; and displaying, via the display generation component, at a third time subsequent to the second time, an updated cycle tracking user interface, including concurrently displaying: a representation of the predicted ovulation date for the user; and an updated fertile window prediction for the user different from the first fertile window prediction for the user.

In accordance with some embodiments, a computer system is described. The computer system is configured to communicate with a display generation component and one or more input devices, and comprises: one or more processors; and memory storing one or more programs configured to be executed by the one or more processors, the one or more programs including instructions for: displaying, via the display generation component, at a first time, a cycle tracking user interface including displaying a first fertile window prediction for a user; receiving body temperature information corresponding to the user; after receiving the body temperature information corresponding to the user, determining, at a second time subsequent to the first time, based on the body temperature information corresponding to the user, a predicted ovulation date for the user; and displaying, via the display generation component, at a third time subsequent to the second time, an updated cycle tracking user interface, including concurrently displaying: a representation of the predicted ovulation date for the user; and an updated fertile window prediction for the user different from the first fertile window prediction for the user.

In accordance with some embodiments, a computer system is described. The computer system is configured to communicate with a display generation component and one or more input devices. The computer system comprises: means for displaying, via the display generation component, at a first time, a cycle tracking user interface including displaying a first fertile window prediction for a user; means for receiving body temperature information corresponding to the user; means for, after receiving the body temperature information corresponding to the user, determining, at a second time subsequent to the first time, based on the body temperature information corresponding to the user, a predicted ovulation date for the user; and means for displaying, via the display generation component, at a third time subsequent to the second time, an updated cycle tracking user interface, including concurrently displaying: a representation of the predicted ovulation date for the user; and an updated fertile window prediction for the user different from the first fertile window prediction for the user.

In accordance with some embodiments, a computer program product is described. The computer program product comprises one or more programs configured to be executed by one or more processors of a computer system that is in communication with a display generation component and one or more input devices, the one or more programs including instructions for: displaying, via the display generation component, at a first time, a cycle tracking user interface including displaying a first fertile window prediction for a user; receiving body temperature information corresponding to the user; after receiving the body temperature information corresponding to the user, determining, at a second time subsequent to the first time, based on the body temperature information corresponding to the user, a predicted ovulation date for the user; and displaying, via the display generation component, at a third time subsequent to the second time, an updated cycle tracking user interface, including concurrently displaying: a representation of the predicted ovulation date for the user; and an updated fertile window prediction for the user different from the first fertile window prediction for the user.

In accordance with some embodiments, a method is described. The method comprises, at a computer system that is in communication with a display generation component and one or more input devices: receiving, via the one or more input devices, menstrual cycle information for a user; and in accordance with a determination that the menstrual cycle information satisfies a first set of criteria, displaying, via the display generation component, a push notification that indicates to the user that the computer system has identified a potential health issue for the user.

In accordance with some embodiments, a non-transitory computer-readable storage medium is described. The non-transitory computer-readable storage medium stores one or more programs configured to be executed by one or more processors of a computer system that is in communication with a display generation component and one or more input devices, the one or more programs including instructions for: receiving, via the one or more input devices, menstrual cycle information for a user; and in accordance with a determination that the menstrual cycle information satisfies a first set of criteria, displaying, via the display generation component, a push notification that indicates to the user that the computer system has identified a potential health issue for the user.

In accordance with some embodiments, a transitory computer-readable storage medium is described. The transitory computer-readable storage medium stores one or more programs configured to be executed by one or more processors of a computer system that is in communication with a display generation component and one or more input devices, the one or more programs including instructions for: receiving, via the one or more input devices, menstrual cycle information for a user; and in accordance with a determination that the menstrual cycle information satisfies a first set of criteria, displaying, via the display generation component, a push notification that indicates to the user that the computer system has identified a potential health issue for the user.

In accordance with some embodiments, a computer system is described. The computer system is configured to communicate with a display generation component and one or more input devices, and comprises: one or more processors; and memory storing one or more programs configured to be executed by the one or more processors, the one or more programs including instructions for: receiving, via the one or more input devices, menstrual cycle information for a user; and in accordance with a determination that the menstrual cycle information satisfies a first set of criteria, displaying, via the display generation component, a push notification that indicates to the user that the computer system has identified a potential health issue for the user.

In accordance with some embodiments, a computer system is described. The computer system is configured to communicate with a display generation component and one or more input devices. The computer system comprises: means for receiving, via the one or more input devices, menstrual cycle information for a user; and means for, in accordance with a determination that the menstrual cycle information satisfies a first set of criteria, displaying, via the display generation component, a push notification that indicates to the user that the computer system has identified a potential health issue for the user.

In accordance with some embodiments, a computer program product is described. The computer program product comprises one or more programs configured to be executed by one or more processors of a computer system that is in communication with a display generation component and one or more input devices, the one or more programs including instructions for: receiving, via the one or more input devices, menstrual cycle information for a user; and in accordance with a determination that the menstrual cycle information satisfies a first set of criteria, displaying, via the display generation component, a push notification that indicates to the user that the computer system has identified a potential health issue for the user.

Executable instructions for performing these functions are, optionally, included in a non-transitory computer-readable storage medium or other computer program product configured for execution by one or more processors. Executable instructions for performing these functions are, optionally, included in a transitory computer-readable storage medium or other computer program product configured for execution by one or more processors.

Thus, devices are provided with faster, more efficient methods and interfaces for tracking health information and for generating related predictions and/or notifications, thereby increasing the effectiveness, efficiency, and user satisfaction with such devices. Such methods and interfaces may complement or replace other methods for tracking health information and for generating related predictions and/or notifications.

### DESCRIPTION OF THE FIGURES

For a better understanding of the various described embodiments, reference should be made to the Description of Embodiments below, in conjunction with the following drawings in which like reference numerals refer to corresponding parts throughout the figures.

FIG. 1A is a block diagram illustrating a portable multifunction device with a touch-sensitive display in accordance with some embodiments.

FIG. 1B is a block diagram illustrating exemplary components for event handling in accordance with some embodiments.

FIG. 2 illustrates a portable multifunction device having a touch screen in accordance with some embodiments.

FIG. 3 is a block diagram of an exemplary multifunction device with a display and a touch-sensitive surface in accordance with some embodiments.

FIG. 4A illustrates an exemplary user interface for a menu of applications on a portable multifunction device in accordance with some embodiments.

FIG. 4B illustrates an exemplary user interface for a multifunction device with a touch-sensitive surface that is separate from the display in accordance with some embodiments.

FIG. 5A illustrates a personal electronic device in accordance with some embodiments.

FIG. 5B is a block diagram illustrating a personal electronic device in accordance with some embodiments.

FIGS. 6A-6Q illustrate exemplary user interfaces for tracking body temperature information, in accordance with some embodiments.

FIG. 7 is a flow diagram illustrating methods for tracking body temperature information, in accordance with some embodiments.

FIGS. 8A-8K illustrate exemplary user interfaces for generating and providing ovulation date predictions, in accordance with some embodiments.

FIG. 9A is a flow diagram illustrating methods for generating and providing ovulation date predictions, in accordance with some embodiments.

FIG. 9B is a flow diagram illustrating methods for generating and providing ovulation date predictions, in accordance with some embodiments.

FIGS. 10A-10N illustrate exemplary user interfaces for generating and providing health-related notifications, in accordance with some embodiments.

FIG. 11 is a flow diagram illustrating methods for generating and providing health-related notifications, in accordance with some embodiments.

### DESCRIPTION OF EMBODIMENTS

The following description sets forth exemplary methods, parameters, and the like. It should be recognized, however, that such description is not intended as a limitation on the scope of the present disclosure but is instead provided as a description of exemplary embodiments.

There is a need for electronic devices that provide efficient methods and interfaces for tracking health information and for generating related predictions and/or notifications. Such techniques can reduce the cognitive burden on a user who track health information, thereby enhancing productivity. Further, such techniques can reduce processor and battery power otherwise wasted on redundant user inputs.

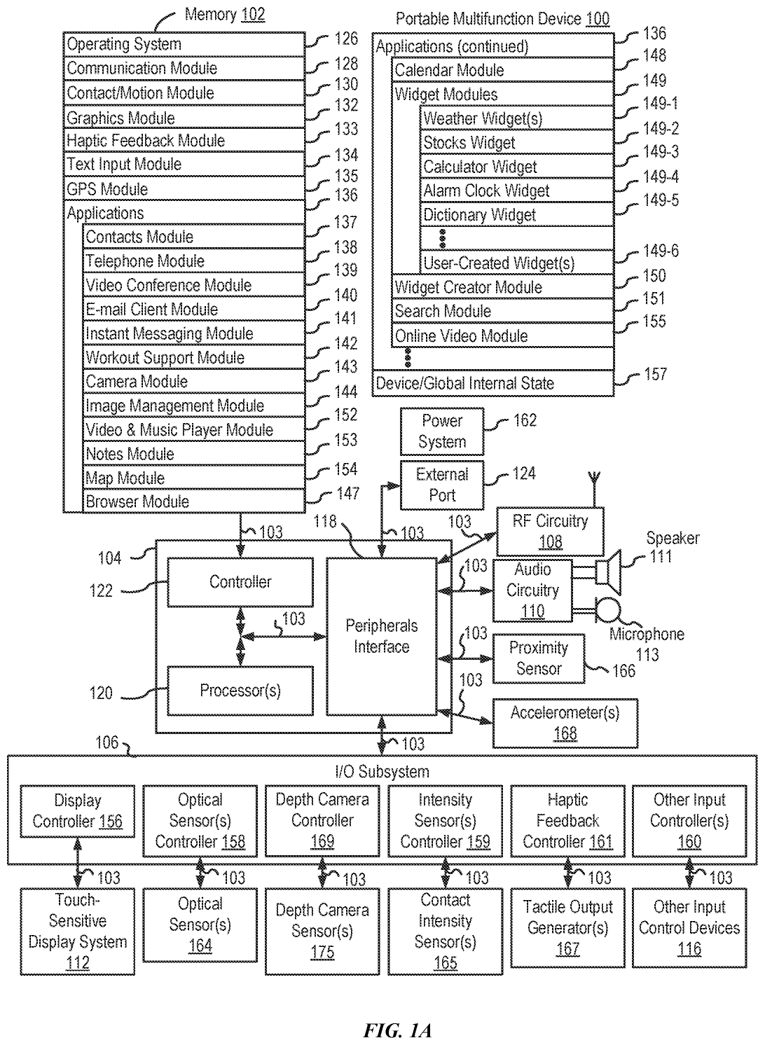

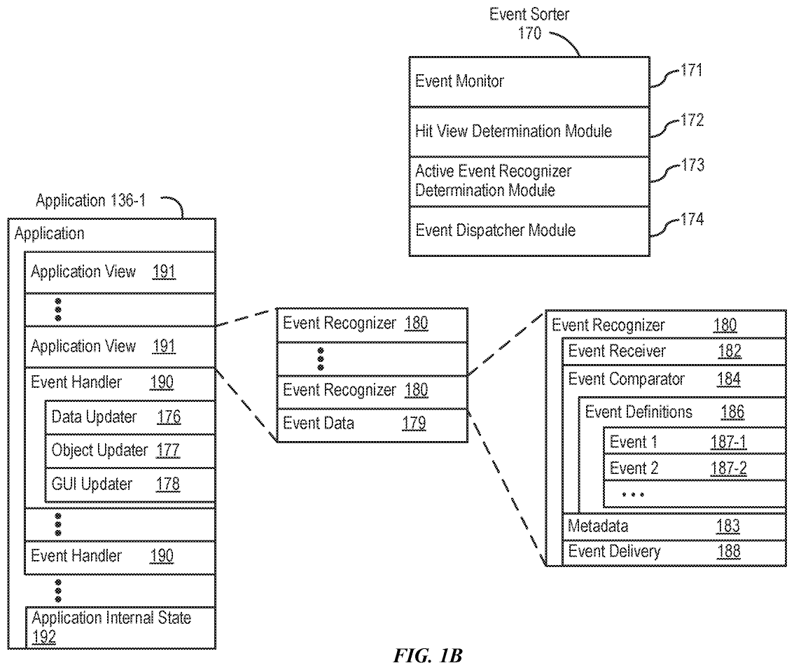

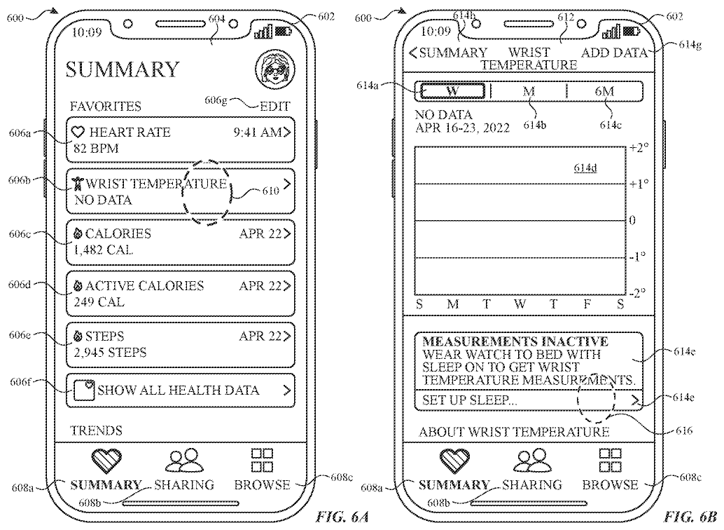

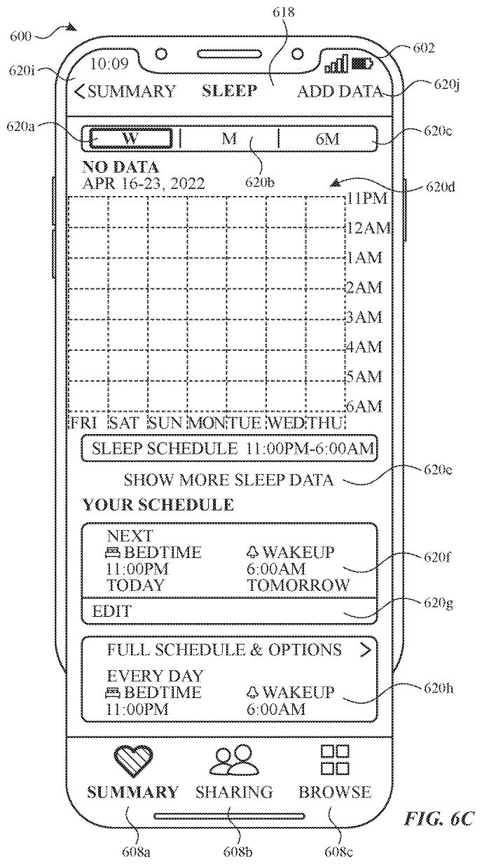

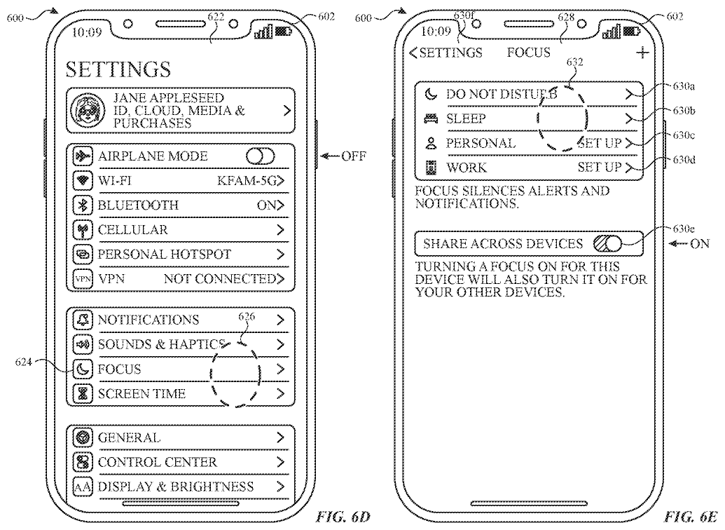

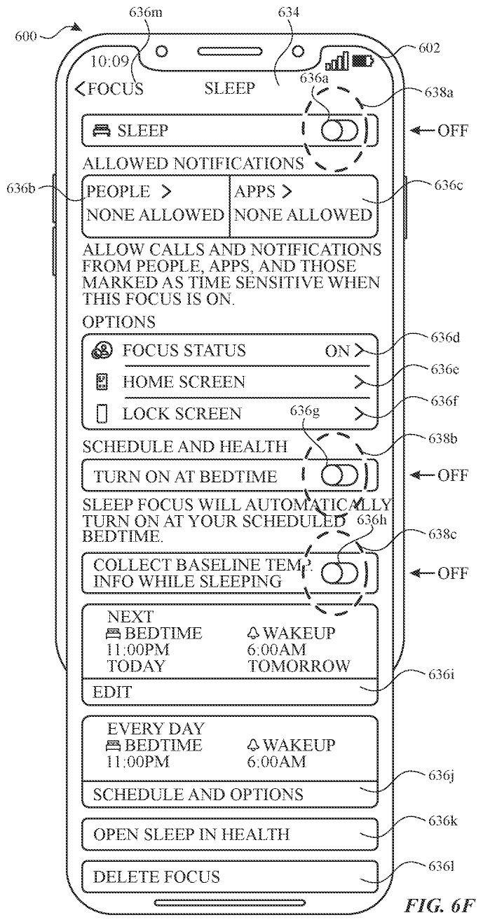

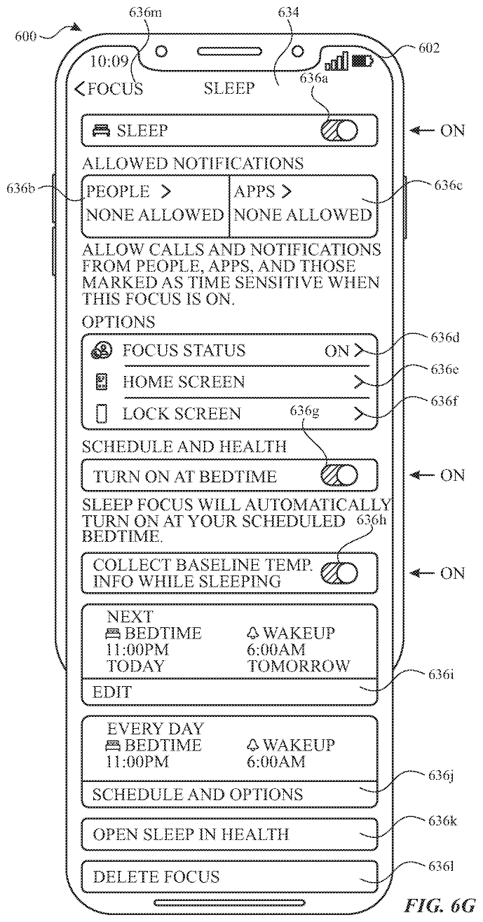

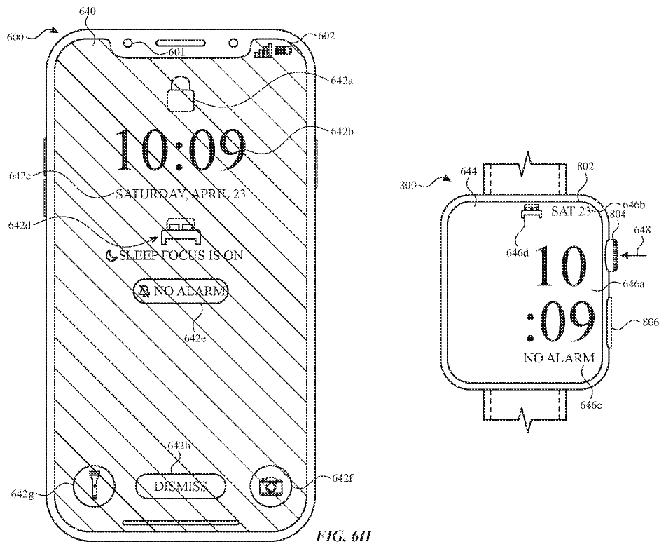

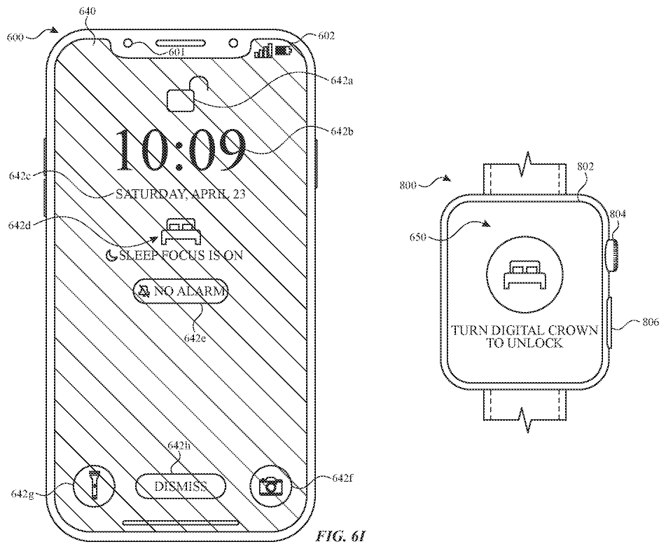

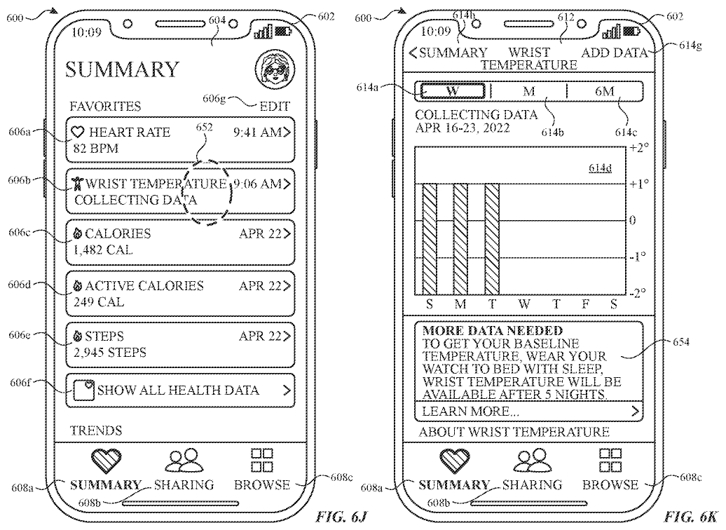

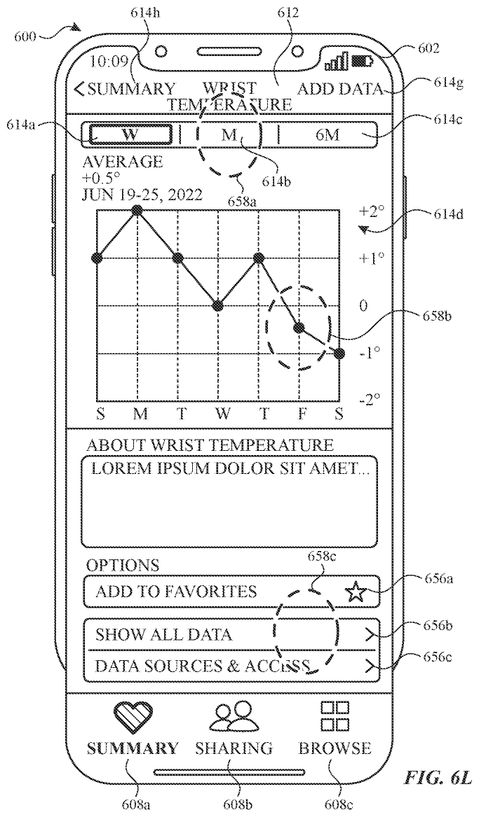

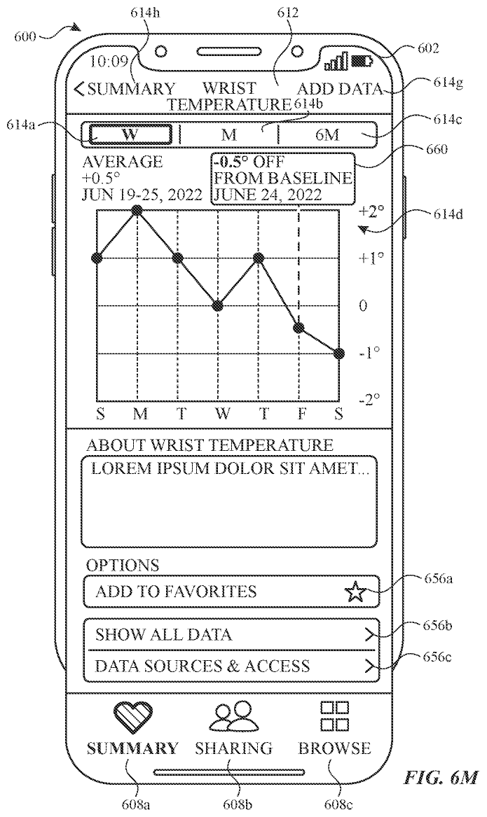

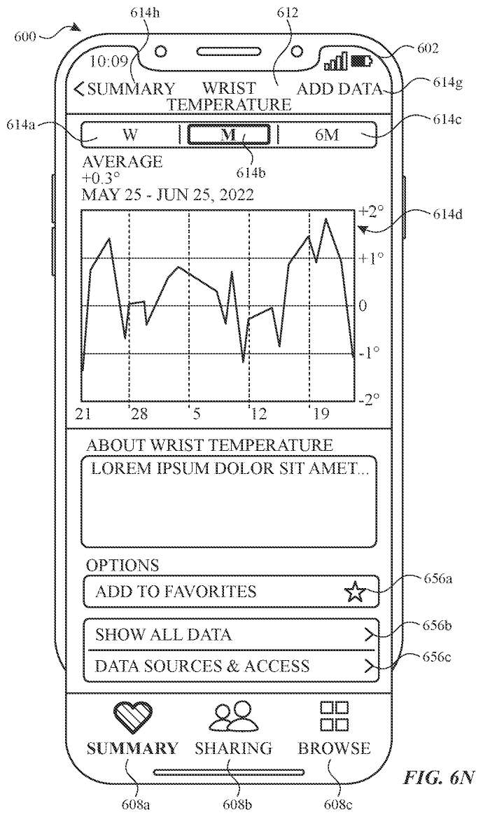

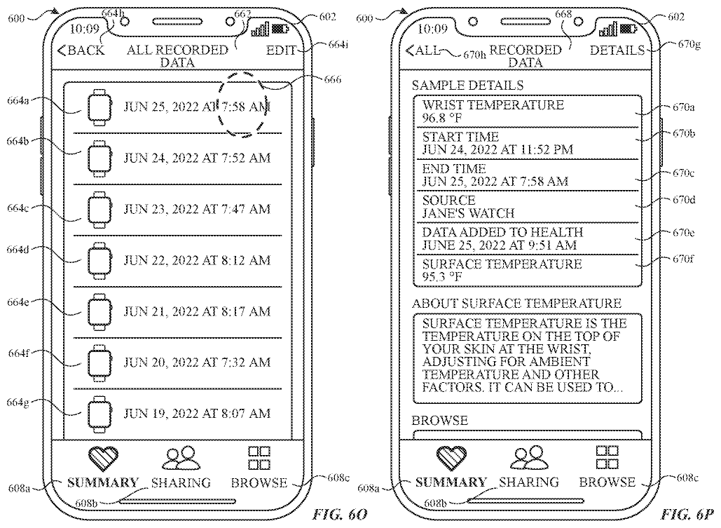

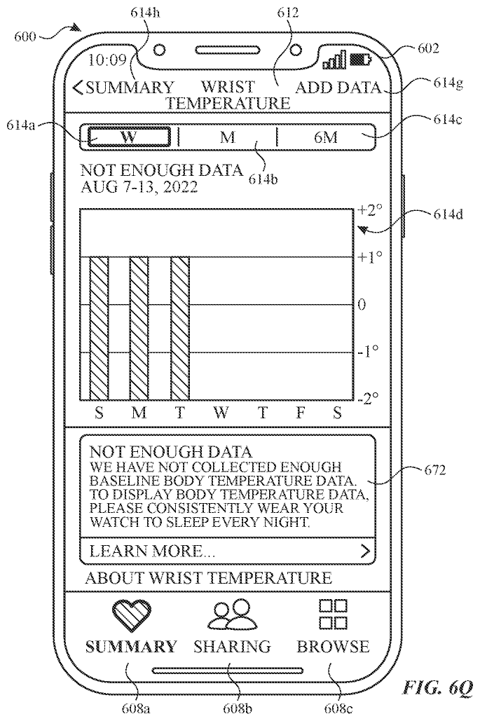

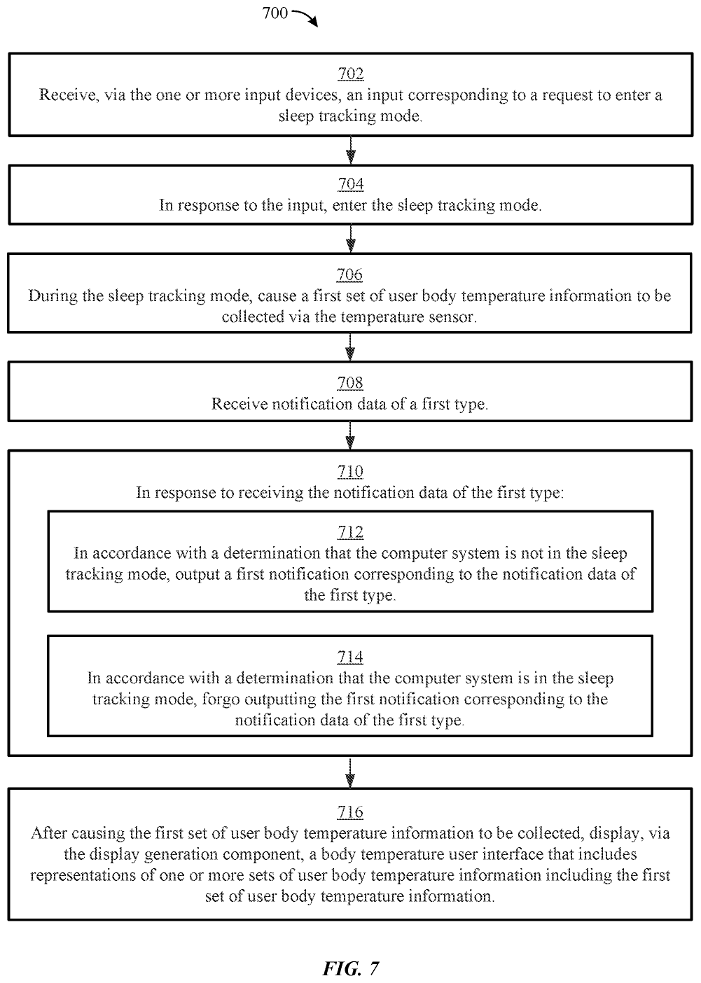

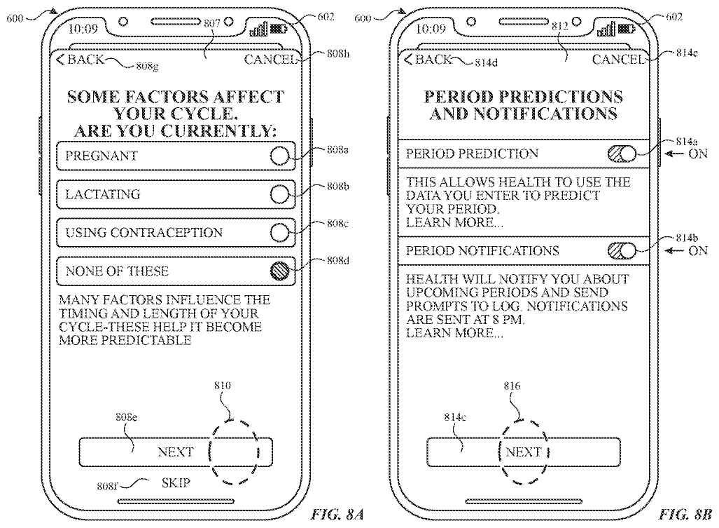

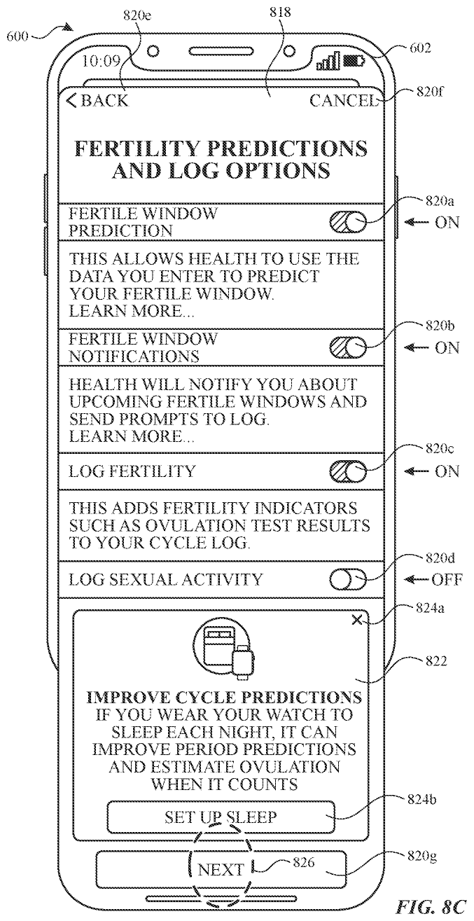

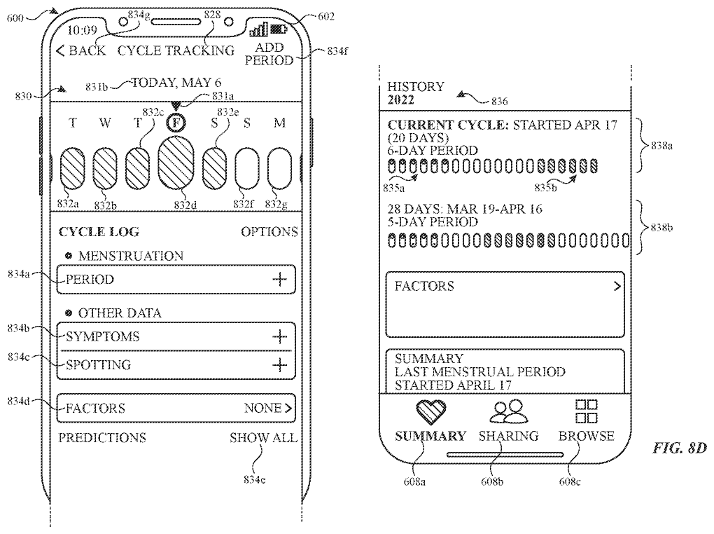

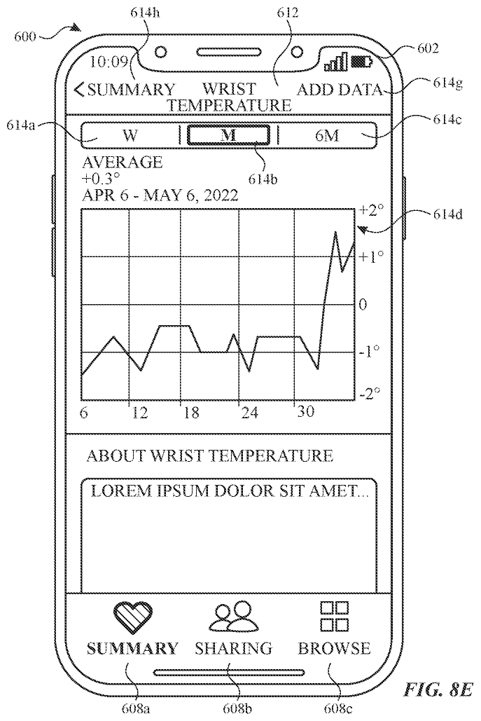

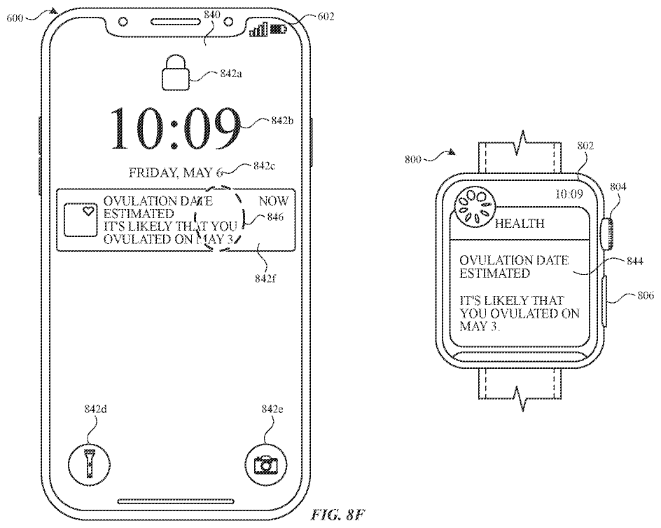

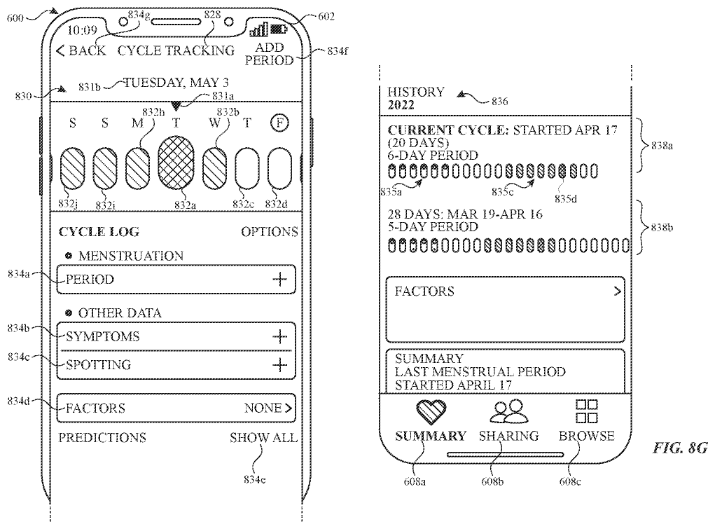

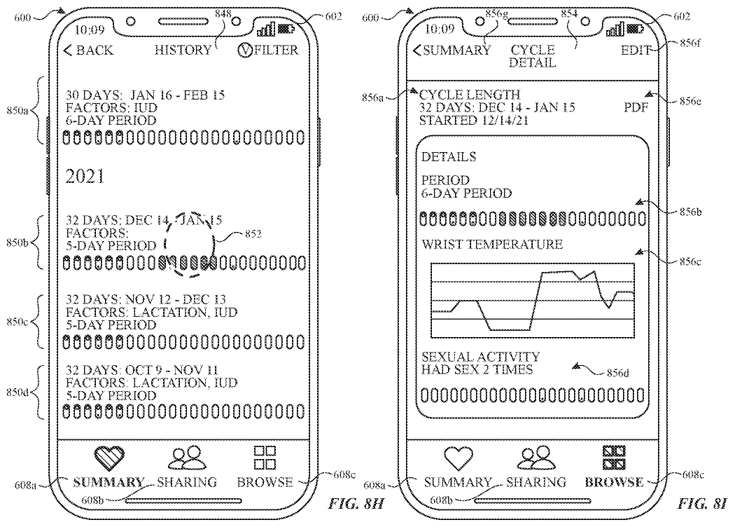

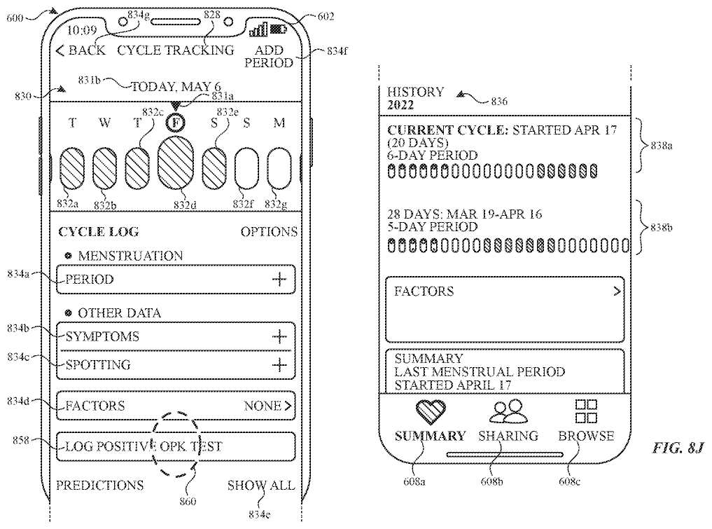

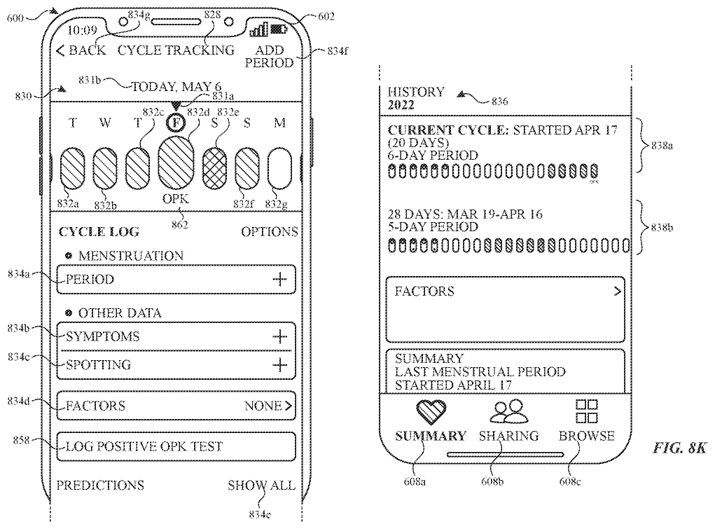

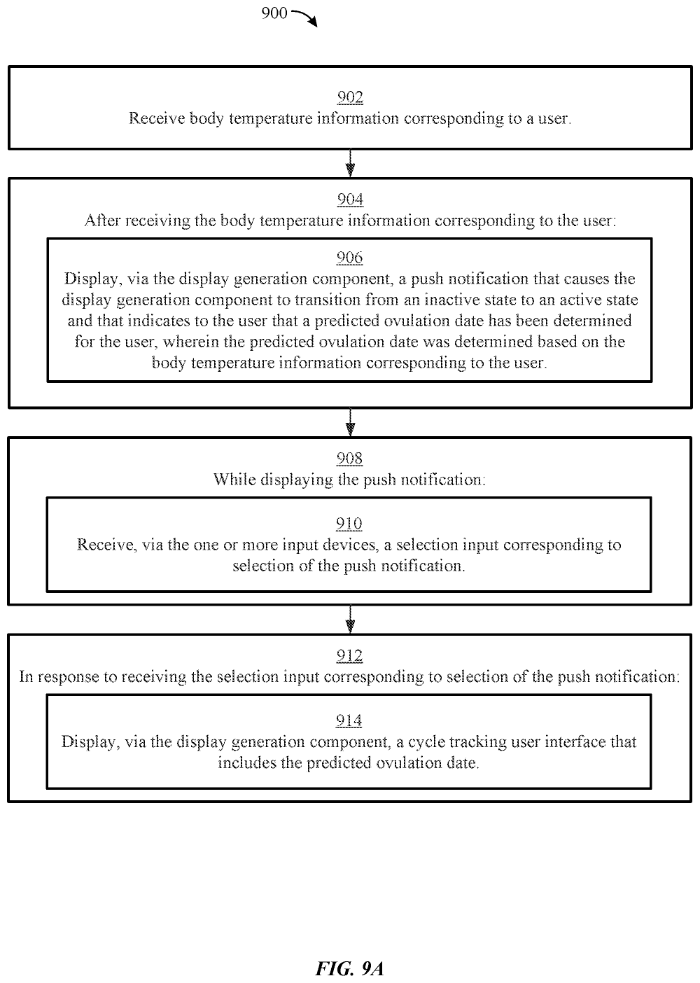

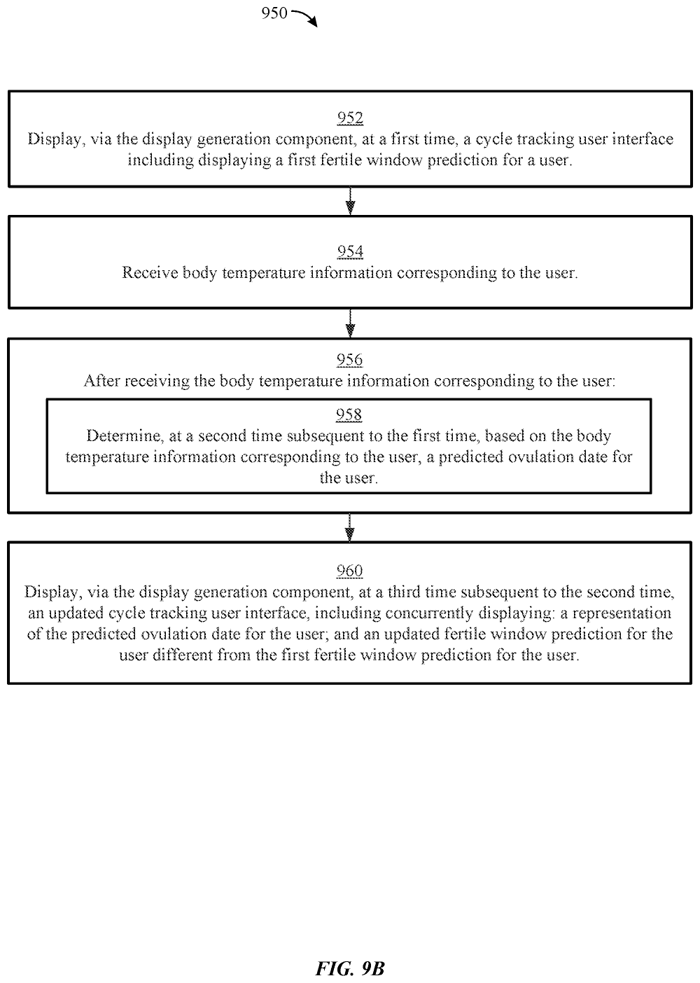

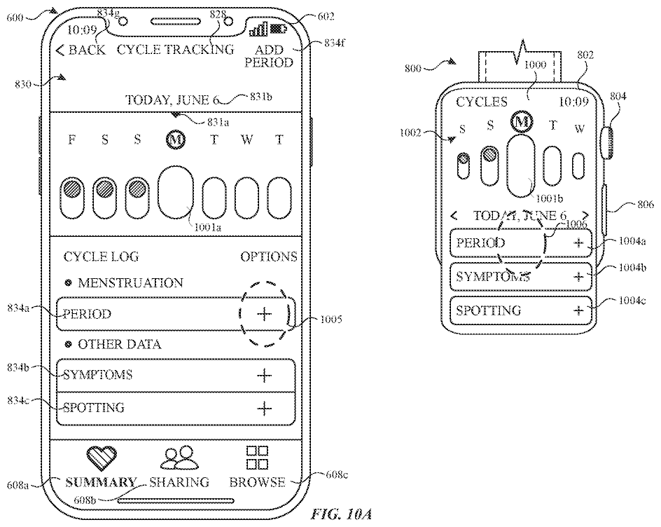

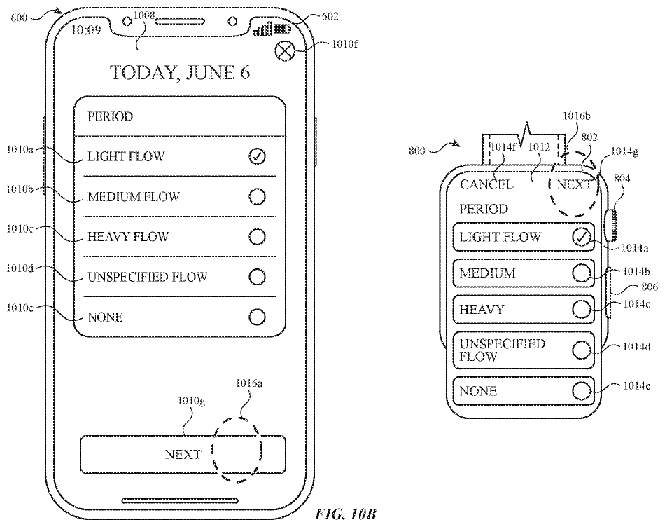

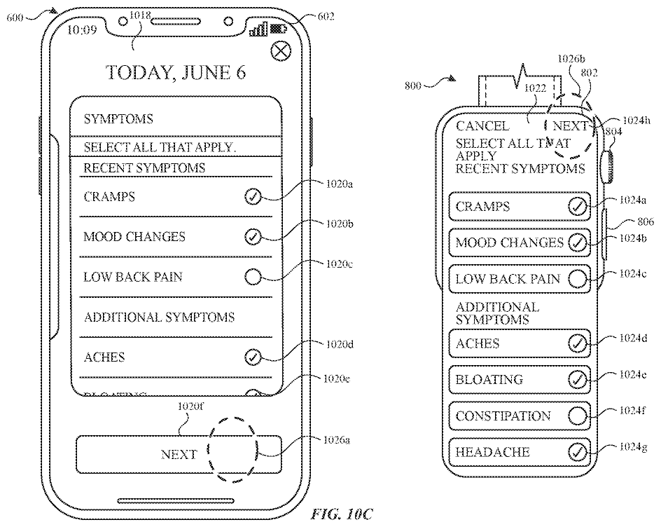

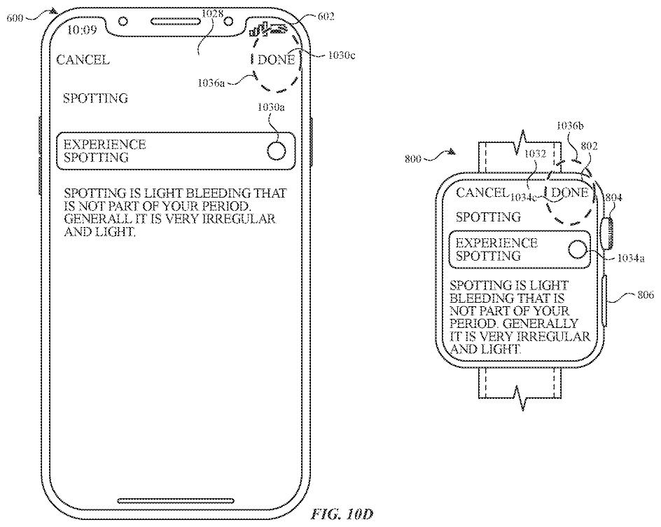

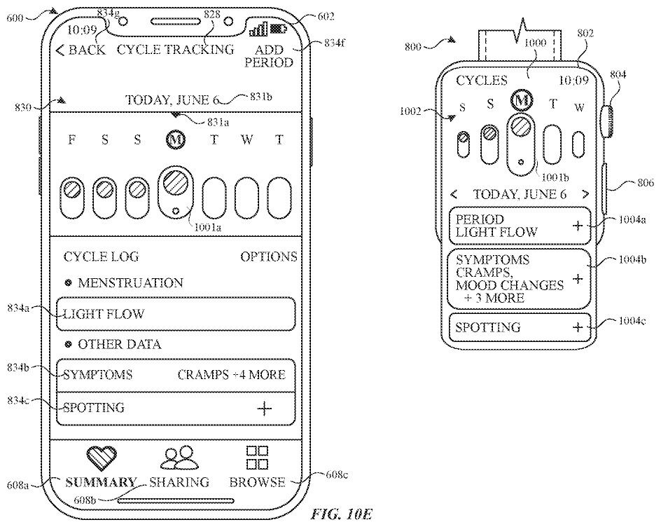

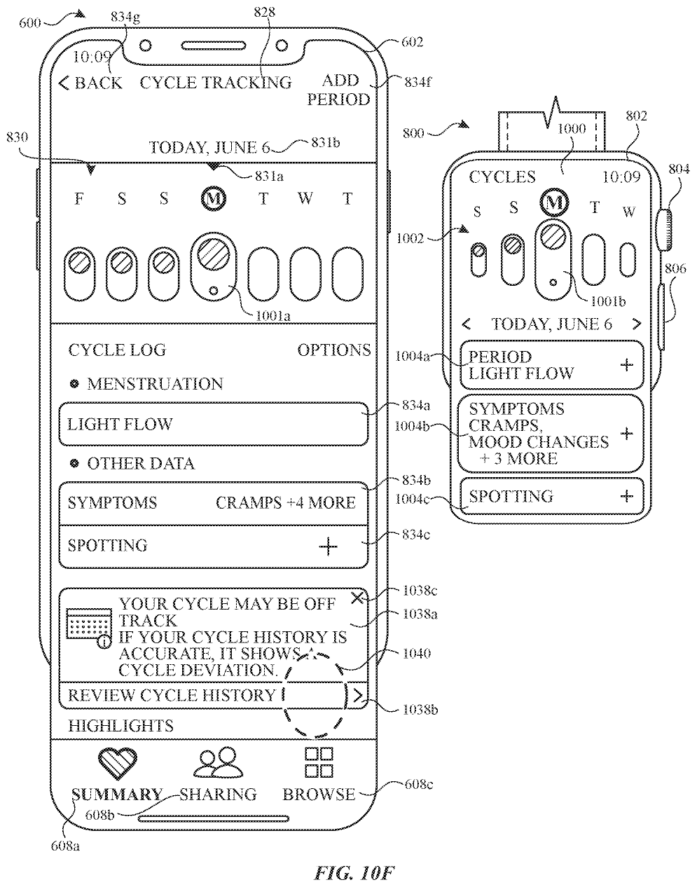

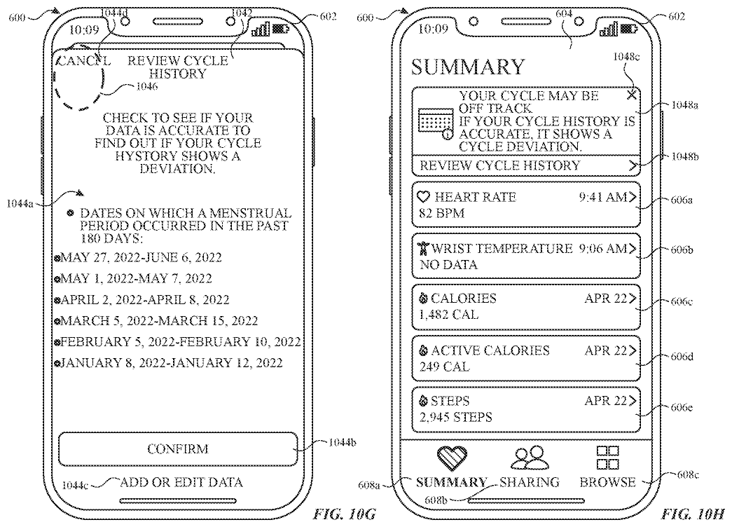

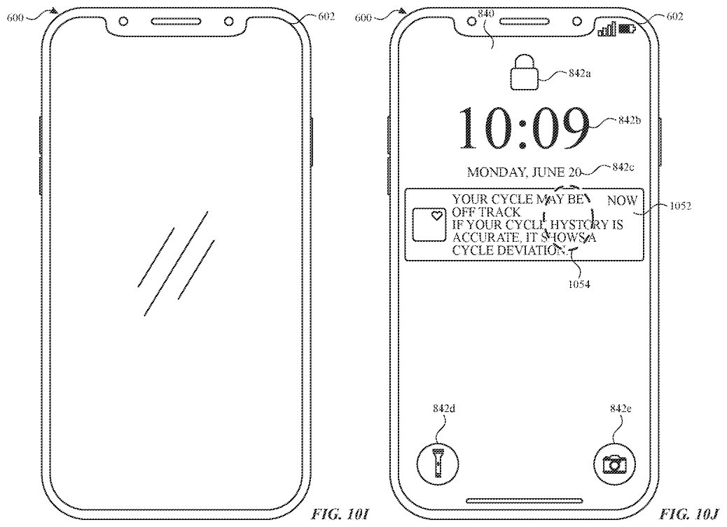

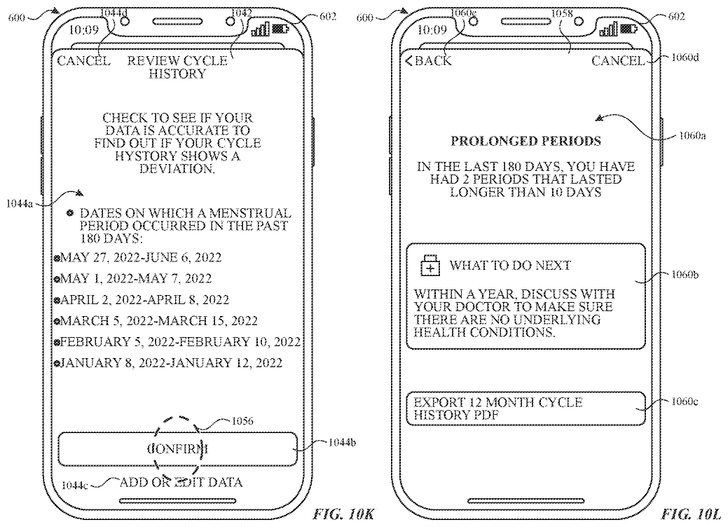

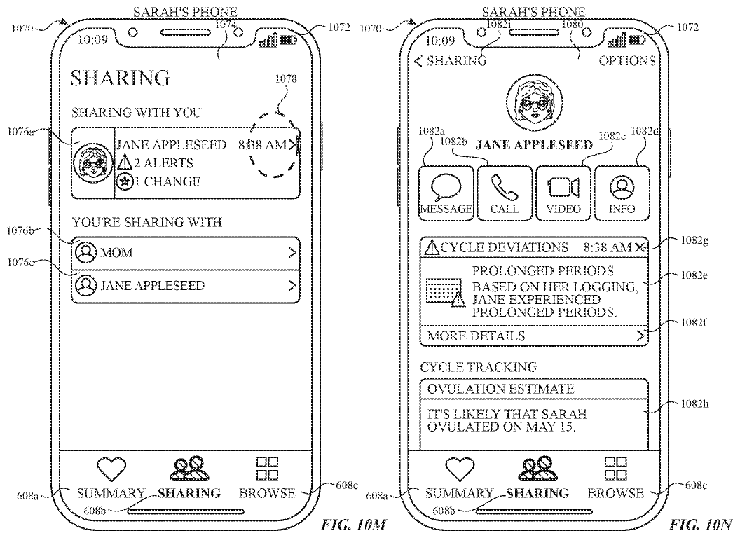

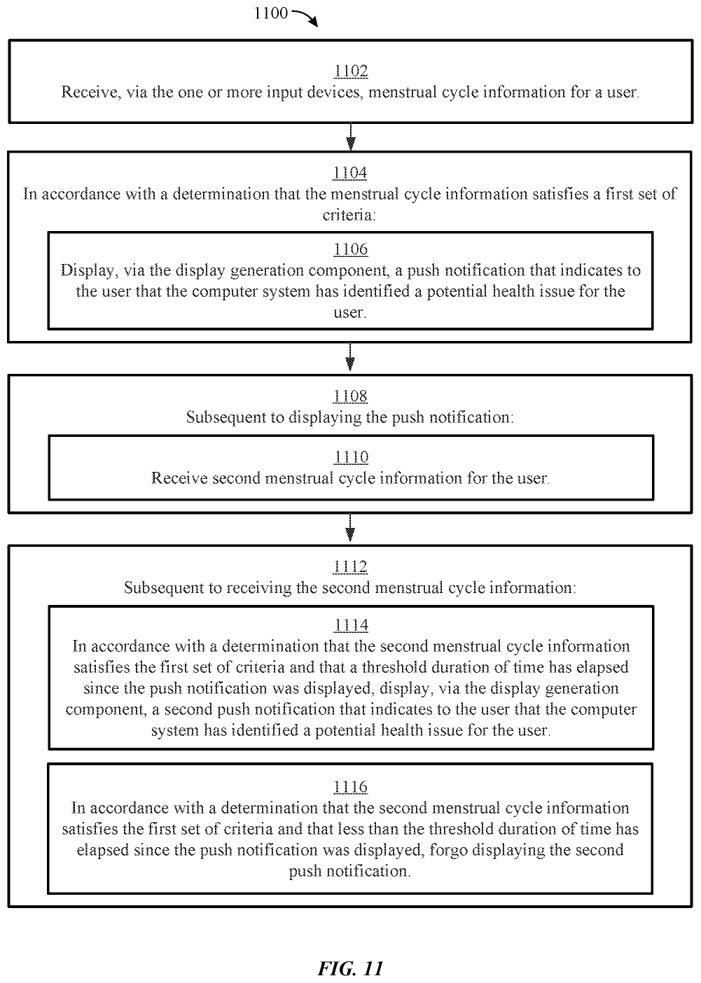

Below, FIGS. 1A-1B, 2, 3, 4A-4B, and 5A-5B provide a description of exemplary devices for performing the techniques for tracking health information and for generating related predictions and/or notifications. FIGS. 6A-6Q illustrate exemplary user interfaces for tracking body temperature information. FIG. 7 is a flow diagram illustrating methods of tracking body temperature information in accordance with some embodiments. The user interfaces in FIGS. 6A-6Q are used to illustrate the processes described below, including the processes in FIG. 7. FIGS. 8A-8K illustrate exemplary user interfaces for generating and providing ovulation date predictions. FIG. 9A is a flow diagram illustrating methods of generating and providing ovulation date predictions in accordance with some embodiments. FIG. 9B is a flow diagram illustrating methods of generating and providing ovulation date predictions in accordance with some embodiments. The user interfaces in FIGS. 8A-8K are used to illustrate the processes described below, including the processes in FIGS. 9A and 9B. FIGS. 10A-10N illustrate exemplary user interfaces for generating and providing health-related notifications. FIG. 11 is a flow diagram illustrating methods of generating and providing health-related notifications in accordance with some embodiments. The user interfaces in FIGS. 10A-10N are used to illustrate the processes described below, including the processes in FIG. 11.

The processes described below enhance the operability of the devices and make the user-device interfaces more efficient (e.g., by helping the user to provide proper inputs and reducing user mistakes when operating/interacting with the device) through various techniques, including by providing improved visual feedback to the user, reducing the number of inputs needed to perform an operation, providing additional control options without cluttering the user interface with additional displayed controls, performing an operation when a set of conditions has been met without requiring further user input, and/or additional techniques. These techniques also reduce power usage and improve battery life of the device by enabling the user to use the device more quickly and efficiently.

In addition, in methods described herein where one or more steps are contingent upon one or more conditions having been met, it should be understood that the described method can be repeated in multiple repetitions so that over the course of the repetitions all of the conditions upon which steps in the method are contingent have been met in different repetitions of the method. For example, if a method requires performing a first step if a condition is satisfied, and a second step if the condition is not satisfied, then a person of ordinary skill would appreciate that the claimed steps are repeated until the condition has been both satisfied and not satisfied, in no particular order. Thus, a method described with one or more steps that are contingent upon one or more conditions having been met could be rewritten as a method that is repeated until each of the conditions described in the method has been met. This, however, is not required of system or computer readable medium claims where the system or computer readable medium contains instructions for performing the contingent operations based on the satisfaction of the corresponding one or more conditions and thus is capable of determining whether the contingency has or has not been satisfied without explicitly repeating steps of a method until all of the conditions upon which steps in the method are contingent have been met. A person having ordinary skill in the art would also understand that, similar to a method with contingent steps, a system or computer readable storage medium can repeat the steps of a method as many times as are needed to ensure that all of the contingent steps have been performed.

Although the following description uses terms “first,” “second,” etc. to describe various elements, these elements should not be limited by the terms. In some embodiments, these terms are used to distinguish one element from another. For example, a first touch could be termed a second touch, and, similarly, a second touch could be termed a first touch, without departing from the scope of the various described embodiments. In some embodiments, the first touch and the second touch are two separate references to the same touch. In some embodiments, the first touch and the second touch are both touches, but they are not the same touch.

The terminology used in the description of the various described embodiments herein is for the purpose of describing particular embodiments only and is not intended to be limiting. As used in the description of the various described embodiments and the appended claims, the singular forms “a,” “an,” and “the” are intended to include the plural forms as well, unless the context clearly indicates otherwise. It will also be understood that the term “and/or” as used herein refers to and encompasses any and all possible combinations of one or more of the associated listed items. It will be further understood that the terms “includes,” “including,” “comprises,” and/or “comprising,” when used in this specification, specify the presence of stated features, integers, steps, operations, elements, and/or components, but do not preclude the presence or addition of one or more other features, integers, steps, operations, elements, components, and/or groups thereof.

The term “if” is, optionally, construed to mean “when” or “upon” or “in response to determining” or “in response to detecting,” depending on the context. Similarly, the phrase “if it is determined” or “if [a stated condition or event] is detected” is, optionally, construed to mean “upon determining” or “in response to determining” or “upon detecting [the stated condition or event]” or “in response to detecting [the stated condition or event],” depending on the context.

Embodiments of electronic devices, user interfaces for such devices, and associated processes for using such devices are described. In some embodiments, the device is a portable communications device, such as a mobile telephone, that also contains other functions, such as PDA and/or music player functions. Exemplary embodiments of portable multifunction devices include, without limitation, the iPhone®, iPod Touch®, and iPad® devices from Apple Inc. of Cupertino, California. Other portable electronic devices, such as laptops or tablet computers with touch-sensitive surfaces (e.g., touch screen displays and/or touchpads), are, optionally, used. It should also be understood that, in some embodiments, the device is not a portable communications device, but is a desktop computer with a touch-sensitive surface (e.g., a touch screen display and/or a touchpad). In some embodiments, the electronic device is a computer system that is in communication (e.g., via wireless communication, via wired communication) with a display generation component. The display generation component is configured to provide visual output, such as display via a CRT display, display via an LED display, or display via image projection. In some embodiments, the display generation component is integrated with the computer system. In some embodiments, the display generation component is separate from the computer system. As used herein, “displaying” content includes causing to display the content (e.g., video data rendered or decoded by display controller 156) by transmitting, via a wired or wireless connection, data (e.g., image data or video data) to an integrated or external display generation component to visually produce the content.

In the discussion that follows, an electronic device that includes a display and a touch-sensitive surface is described. It should be understood, however, that the electronic device optionally includes one or more other physical user-interface devices, such as a physical keyboard, a mouse, and/or a joystick.

The device typically supports a variety of applications, such as one or more of the following: a drawing application, a presentation application, a word processing application, a website creation application, a disk authoring application, a spreadsheet application, a gaming application, a telephone application, a video conferencing application, an e-mail application, an instant messaging application, a workout support application, a photo management application, a digital camera application, a digital video camera application, a web browsing application, a digital music player application, and/or a digital video player application.

The various applications that are executed on the device optionally use at least one common physical user-interface device, such as the touch-sensitive surface. One or more functions of the touch-sensitive surface as well as corresponding information displayed on the device are, optionally, adjusted and/or varied from one application to the next and/or within a respective application. In this way, a common physical architecture (such as the touch-sensitive surface) of the device optionally supports the variety of applications with user interfaces that are intuitive and transparent to the user.

Attention is now directed toward embodiments of portable devices with touch-sensitive displays. FIG. 1A is a block diagram illustrating portable multifunction device 100 with touch-sensitive display system 112 in accordance with some embodiments. Touch-sensitive display 112 is sometimes called a “touch screen” for convenience and is sometimes known as or called a “touch-sensitive display system.” Device 100 includes memory 102 (which optionally includes one or more computer-readable storage mediums), memory controller 122, one or more processing units (CPUs) 120, peripherals interface 118, RF circuitry 108, audio circuitry 110, speaker 111, microphone 113, input/output (I/O) subsystem 106, other input control devices 116, and external port 124. Device 100 optionally includes one or more optical sensors 164. Device 100 optionally includes one or more contact intensity sensors 165 for detecting intensity of contacts on device 100 (e.g., a touch-sensitive surface such as touch-sensitive display system 112 of device 100). Device 100 optionally includes one or more tactile output generators 167 for generating tactile outputs on device 100 (e.g., generating tactile outputs on a touch-sensitive surface such as touch-sensitive display system 112 of device 100 or touchpad 355 of device 300). These components optionally communicate over one or more communication buses or signal lines 103.

As used in the specification and claims, the term “intensity” of a contact on a touch-sensitive surface refers to the force or pressure (force per unit area) of a contact (e.g., a finger contact) on the touch-sensitive surface, or to a substitute (proxy) for the force or pressure of a contact on the touch-sensitive surface. The intensity of a contact has a range of values that includes at least four distinct values and more typically includes hundreds of distinct values (e.g., at least 256). Intensity of a contact is, optionally, determined (or measured) using various approaches and various sensors or combinations of sensors. For example, one or more force sensors underneath or adjacent to the touch-sensitive surface are, optionally, used to measure force at various points on the touch-sensitive surface. In some implementations, force measurements from multiple force sensors are combined (e.g., a weighted average) to determine an estimated force of a contact. Similarly, a pressure-sensitive tip of a stylus is, optionally, used to determine a pressure of the stylus on the touch-sensitive surface. Alternatively, the size of the contact area detected on the touch-sensitive surface and/or changes thereto, the capacitance of the touch-sensitive surface proximate to the contact and/or changes thereto, and/or the resistance of the touch-sensitive surface proximate to the contact and/or changes thereto are, optionally, used as a substitute for the force or pressure of the contact on the touch-sensitive surface. In some implementations, the substitute measurements for contact force or pressure are used directly to determine whether an intensity threshold has been exceeded (e.g., the intensity threshold is described in units corresponding to the substitute measurements). In some implementations, the substitute measurements for contact force or pressure are converted to an estimated force or pressure, and the estimated force or pressure is used to determine whether an intensity threshold has been exceeded (e.g., the intensity threshold is a pressure threshold measured in units of pressure). Using the intensity of a contact as an attribute of a user input allows for user access to additional device functionality that may otherwise not be accessible by the user on a reduced-size device with limited real estate for displaying affordances (e.g., on a touch-sensitive display) and/or receiving user input (e.g., via a touch-sensitive display, a touch-sensitive surface, or a physical/mechanical control such as a knob or a button).

As used in the specification and claims, the term “tactile output” refers to physical displacement of a device relative to a previous position of the device, physical displacement of a component (e.g., a touch-sensitive surface) of a device relative to another component (e.g., housing) of the device, or displacement of the component relative to a center of mass of the device that will be detected by a user with the user's sense of touch. For example, in situations where the device or the component of the device is in contact with a surface of a user that is sensitive to touch (e.g., a finger, palm, or other part of a user's hand), the tactile output generated by the physical displacement will be interpreted by the user as a tactile sensation corresponding to a perceived change in physical characteristics of the device or the component of the device. For example, movement of a touch-sensitive surface (e.g., a touch-sensitive display or trackpad) is, optionally, interpreted by the user as a “down click” or “up click” of a physical actuator button. In some cases, a user will feel a tactile sensation such as an “down click” or “up click” even when there is no movement of a physical actuator button associated with the touch-sensitive surface that is physically pressed (e.g., displaced) by the user's movements. As another example, movement of the touch-sensitive surface is, optionally, interpreted or sensed by the user as “roughness” of the touch-sensitive surface, even when there is no change in smoothness of the touch-sensitive surface. While such interpretations of touch by a user will be subject to the individualized sensory perceptions of the user, there are many sensory perceptions of touch that are common to a large majority of users. Thus, when a tactile output is described as corresponding to a particular sensory perception of a user (e.g., an “up click,” a “down click,” “roughness”), unless otherwise stated, the generated tactile output corresponds to physical displacement of the device or a component thereof that will generate the described sensory perception for a typical (or average) user.

It should be appreciated that device 100 is only one example of a portable multifunction device, and that device 100 optionally has more or fewer components than shown, optionally combines two or more components, or optionally has a different configuration or arrangement of the components. The various components shown in FIG. 1A are implemented in hardware, software, or a combination of both hardware and software, including one or more signal processing and/or application-specific integrated circuits.

Memory 102 optionally includes high-speed random access memory and optionally also includes non-volatile memory, such as one or more magnetic disk storage devices, flash memory devices, or other non-volatile solid-state memory devices. Memory controller 122 optionally controls access to memory 102 by other components of device 100.

Peripherals interface 118 can be used to couple input and output peripherals of the device to CPU 120 and memory 102. The one or more processors 120 run or execute various software programs (such as computer programs (e.g., including instructions)) and/or sets of instructions stored in memory 102 to perform various functions for device 100 and to process data. In some embodiments, peripherals interface 118, CPU 120, and memory controller 122 are, optionally, implemented on a single chip, such as chip 104. In some other embodiments, they are, optionally, implemented on separate chips.

RF (radio frequency) circuitry 108 receives and sends RF signals, also called electromagnetic signals. RF circuitry 108 converts electrical signals to/from electromagnetic signals and communicates with communications networks and other communications devices via the electromagnetic signals. RF circuitry 108 optionally includes well-known circuitry for performing these functions, including but not limited to an antenna system, an RF transceiver, one or more amplifiers, a tuner, one or more oscillators, a digital signal processor, a CODEC chipset, a subscriber identity module (SIM) card, memory, and so forth. RF circuitry 108 optionally communicates with networks, such as the Internet, also referred to as the World Wide Web (WWW), an intranet and/or a wireless network, such as a cellular telephone network, a wireless local area network (LAN) and/or a metropolitan area network (MAN), and other devices by wireless communication. The RF circuitry 108 optionally includes well-known circuitry for detecting near field communication (NFC) fields, such as by a short-range communication radio. The wireless communication optionally uses any of a plurality of communications standards, protocols, and technologies, including but not limited to Global System for Mobile Communications (GSM), Enhanced Data GSM Environment (EDGE), high-speed downlink packet access (HSDPA), high-speed uplink packet access (HSUPA), Evolution, Data-Only (EV-DO), HSPA, HSPA+, Dual-Cell HSPA (DC-HSPDA), long term evolution (LTE), near field communication (NFC), wideband code division multiple access (W-CDMA), code division multiple access (CDMA), time division multiple access (TDMA), Bluetooth, Bluetooth Low Energy (BTLE), Wireless Fidelity (Wi-Fi) (e.g., IEEE 802.11a, IEEE 802.11b, IEEE 802.11g, IEEE 802.11n, and/or IEEE 802.11ac), voice over Internet Protocol (VoIP), Wi-MAX, a protocol for e-mail (e.g., Internet message access protocol (IMAP) and/or post office protocol (POP)), instant messaging (e.g., extensible messaging and presence protocol (XMPP), Session Initiation Protocol for Instant Messaging and Presence Leveraging Extensions (SIMPLE), Instant Messaging and Presence Service (IMPS)), and/or Short Message Service (SMS), or any other suitable communication protocol, including communication protocols not yet developed as of the filing date of this document.

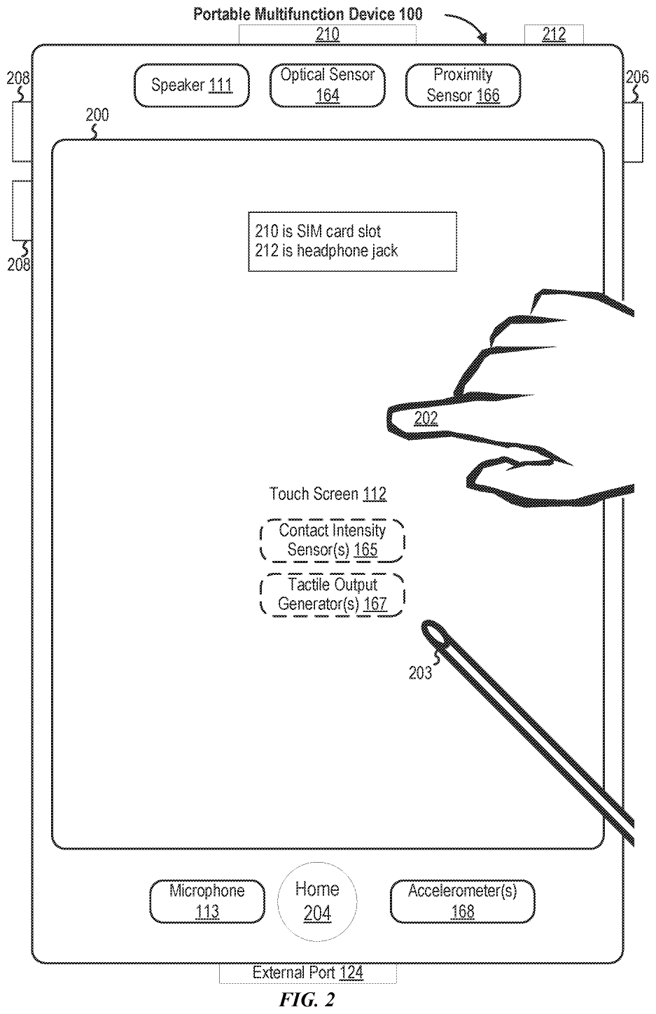

Audio circuitry 110, speaker 111, and microphone 113 provide an audio interface between a user and device 100. Audio circuitry 110 receives audio data from peripherals interface 118, converts the audio data to an electrical signal, and transmits the electrical signal to speaker 111. Speaker 111 converts the electrical signal to human-audible sound waves. Audio circuitry 110 also receives electrical signals converted by microphone 113 from sound waves. Audio circuitry 110 converts the electrical signal to audio data and transmits the audio data to peripherals interface 118 for processing. Audio data is, optionally, retrieved from and/or transmitted to memory 102 and/or RF circuitry 108 by peripherals interface 118. In some embodiments, audio circuitry 110 also includes a headset jack (e.g., 212, FIG. 2). The headset jack provides an interface between audio circuitry 110 and removable audio input/output peripherals, such as output-only headphones or a headset with both output (e.g., a headphone for one or both ears) and input (e.g., a microphone).

I/O subsystem 106 couples input/output peripherals on device 100, such as touch screen 112 and other input control devices 116, to peripherals interface 118. I/O subsystem 106 optionally includes display controller 156, optical sensor controller 158, depth camera controller 169, intensity sensor controller 159, haptic feedback controller 161, and one or more input controllers 160 for other input or control devices. The one or more input controllers 160 receive/send electrical signals from/to other input control devices 116. The other input control devices 116 optionally include physical buttons (e.g., push buttons, rocker buttons, etc.), dials, slider switches, joysticks, click wheels, and so forth. In some embodiments, input controller(s) 160 are, optionally, coupled to any (or none) of the following: a keyboard, an infrared port, a USB port, and a pointer device such as a mouse. The one or more buttons (e.g., 208, FIG. 2) optionally include an up/down button for volume control of speaker 111 and/or microphone 113. The one or more buttons optionally include a push button (e.g., 206, FIG. 2). In some embodiments, the electronic device is a computer system that is in communication (e.g., via wireless communication, via wired communication) with one or more input devices. In some embodiments, the one or more input devices include a touch-sensitive surface (e.g., a trackpad, as part of a touch-sensitive display). In some embodiments, the one or more input devices include one or more camera sensors (e.g., one or more optical sensors 164 and/or one or more depth camera sensors 175), such as for tracking a user's gestures (e.g., hand gestures and/or air gestures) as input. In some embodiments, the one or more input devices are integrated with the computer system. In some embodiments, the one or more input devices are separate from the computer system. In some embodiments, an air gesture is a gesture that is detected without the user touching an input element that is part of the device (or independently of an input element that is a part of the device) and is based on detected motion of a portion of the user's body through the air including motion of the user's body relative to an absolute reference (e.g., an angle of the user's arm relative to the ground or a distance of the user's hand relative to the ground), relative to another portion of the user's body (e.g., movement of a hand of the user relative to a shoulder of the user, movement of one hand of the user relative to another hand of the user, and/or movement of a finger of the user relative to another finger or portion of a hand of the user), and/or absolute motion of a portion of the user's body (e.g., a tap gesture that includes movement of a hand in a predetermined pose by a predetermined amount and/or speed, or a shake gesture that includes a predetermined speed or amount of rotation of a portion of the user's body).

A quick press of the push button optionally disengages a lock of touch screen 112 or optionally begins a process that uses gestures on the touch screen to unlock the device, as described in U.S. patent application Ser. No. 11/322,549, “Unlocking a Device by Performing Gestures on an Unlock Image,” filed Dec. 23, 2005, U.S. Pat. No. 7,657,849, which is hereby incorporated by reference in its entirety. A longer press of the push button (e.g., 206) optionally turns power to device 100 on or off. The functionality of one or more of the buttons are, optionally, user-customizable. Touch screen 112 is used to implement virtual or soft buttons and one or more soft keyboards.

Touch-sensitive display 112 provides an input interface and an output interface between the device and a user. Display controller 156 receives and/or sends electrical signals from/to touch screen 112. Touch screen 112 displays visual output to the user. The visual output optionally includes graphics, text, icons, video, and any combination thereof (collectively termed “graphics”). In some embodiments, some or all of the visual output optionally corresponds to user-interface objects.

Touch screen 112 has a touch-sensitive surface, sensor, or set of sensors that accepts input from the user based on haptic and/or tactile contact. Touch screen 112 and display controller 156 (along with any associated modules and/or sets of instructions in memory 102) detect contact (and any movement or breaking of the contact) on touch screen 112 and convert the detected contact into interaction with user-interface objects (e.g., one or more soft keys, icons, web pages, or images) that are displayed on touch screen 112. In an exemplary embodiment, a point of contact between touch screen 112 and the user corresponds to a finger of the user.

Touch screen 112 optionally uses LCD (liquid crystal display) technology, LPD (light emitting polymer display) technology, or LED (light emitting diode) technology, although other display technologies are used in other embodiments. Touch screen 112 and display controller 156 optionally detect contact and any movement or breaking thereof using any of a plurality of touch sensing technologies now known or later developed, including but not limited to capacitive, resistive, infrared, and surface acoustic wave technologies, as well as other proximity sensor arrays or other elements for determining one or more points of contact with touch screen 112. In an exemplary embodiment, projected mutual capacitance sensing technology is used, such as that found in the iPhone® and iPod Touch® from Apple Inc. of Cupertino, California.

A touch-sensitive display in some embodiments of touch screen 112 is, optionally, analogous to the multi-touch sensitive touchpads described in the following U.S. Pat. No. 6,323,846 (Westerman et al.), U.S. Pat. No. 6,570,557 (Westerman et al.), and/or U.S. Pat. No. 6,677,932 (Westerman), and/or U.S. Patent Publication 2002/0015024A1, each of which is hereby incorporated by reference in its entirety. However, touch screen 112 displays visual output from device 100, whereas touch-sensitive touchpads do not provide visual output.

A touch-sensitive display in some embodiments of touch screen 112 is described in the following applications: (1) U.S. patent application Ser. No. 11/381,313, “Multipoint Touch Surface Controller,” filed May 2, 2006; (2) U.S. patent application Ser. No. 10/840,862, “Multipoint Touchscreen,” filed May 6, 2004; (3) U.S. patent application Ser. No. 10/903,964, “Gestures For Touch Sensitive Input Devices,” filed Jul. 30, 2004; (4) U.S. patent application Ser. No. 11/048,264, “Gestures For Touch Sensitive Input Devices,” filed Jan. 31, 2005; (5) U.S. patent application Ser. No. 11/038,590, “Mode-Based Graphical User Interfaces For Touch Sensitive Input Devices,” filed Jan. 18, 2005; (6) U.S. patent application Ser. No. 11/228,758, “Virtual Input Device Placement On A Touch Screen User Interface,” filed Sep. 16, 2005; (7) U.S. patent application Ser. No. 11/228,700, “Operation Of A Computer With A Touch Screen Interface,” filed Sep. 16, 2005; (8) U.S. patent application Ser. No. 11/228,737, “Activating Virtual Keys Of A Touch-Screen Virtual Keyboard,” filed Sep. 16, 2005; and (9) U.S. patent application Ser. No. 11/367,749, “Multi-Functional Hand-Held Device,” filed Mar. 3, 2006. All of these applications are incorporated by reference herein in their entirety.

Touch screen 112 optionally has a video resolution in excess of 100 dpi. In some embodiments, the touch screen has a video resolution of approximately 160 dpi. The user optionally makes contact with touch screen 112 using any suitable object or appendage, such as a stylus, a finger, and so forth. In some embodiments, the user interface is designed to work primarily with finger-based contacts and gestures, which can be less precise than stylus-based input due to the larger area of contact of a finger on the touch screen. In some embodiments, the device translates the rough finger-based input into a precise pointer/cursor position or command for performing the actions desired by the user.

In some embodiments, in addition to the touch screen, device 100 optionally includes a touchpad for activating or deactivating particular functions. In some embodiments, the touchpad is a touch-sensitive area of the device that, unlike the touch screen, does not display visual output. The touchpad is, optionally, a touch-sensitive surface that is separate from touch screen 112 or an extension of the touch-sensitive surface formed by the touch screen.

Device 100 also includes power system 162 for powering the various components. Power system 162 optionally includes a power management system, one or more power sources (e.g., battery, alternating current (AC)), a recharging system, a power failure detection circuit, a power converter or inverter, a power status indicator (e.g., a light-emitting diode (LED)) and any other components associated with the generation, management and distribution of power in portable devices.

Device 100 optionally also includes one or more optical sensors 164. FIG. 1A shows an optical sensor coupled to optical sensor controller 158 in I/O subsystem 106. Optical sensor 164 optionally includes charge-coupled device (CCD) or complementary metal-oxide semiconductor (CMOS) phototransistors. Optical sensor 164 receives light from the environment, projected through one or more lenses, and converts the light to data representing an image. In conjunction with imaging module 143 (also called a camera module), optical sensor 164 optionally captures still images or video. In some embodiments, an optical sensor is located on the back of device 100, opposite touch screen display 112 on the front of the device so that the touch screen display is enabled for use as a viewfinder for still and/or video image acquisition. In some embodiments, an optical sensor is located on the front of the device so that the user's image is, optionally, obtained for video conferencing while the user views the other video conference participants on the touch screen display. In some embodiments, the position of optical sensor 164 can be changed by the user (e.g., by rotating the lens and the sensor in the device housing) so that a single optical sensor 164 is used along with the touch screen display for both video conferencing and still and/or video image acquisition.

Device 100 optionally also includes one or more depth camera sensors 175. FIG. 1A shows a depth camera sensor coupled to depth camera controller 169 in I/O subsystem 106. Depth camera sensor 175 receives data from the environment to create a three dimensional model of an object (e.g., a face) within a scene from a viewpoint (e.g., a depth camera sensor). In some embodiments, in conjunction with imaging module 143 (also called a camera module), depth camera sensor 175 is optionally used to determine a depth map of different portions of an image captured by the imaging module 143. In some embodiments, a depth camera sensor is located on the front of device 100 so that the user's image with depth information is, optionally, obtained for video conferencing while the user views the other video conference participants on the touch screen display and to capture selfies with depth map data. In some embodiments, the depth camera sensor 175 is located on the back of device, or on the back and the front of the device 100. In some embodiments, the position of depth camera sensor 175 can be changed by the user (e.g., by rotating the lens and the sensor in the device housing) so that a depth camera sensor 175 is used along with the touch screen display for both video conferencing and still and/or video image acquisition.

Device 100 optionally also includes one or more contact intensity sensors 165. FIG. 1A shows a contact intensity sensor coupled to intensity sensor controller 159 in I/O subsystem 106. Contact intensity sensor 165 optionally includes one or more piezoresistive strain gauges, capacitive force sensors, electric force sensors, piezoelectric force sensors, optical force sensors, capacitive touch-sensitive surfaces, or other intensity sensors (e.g., sensors used to measure the force (or pressure) of a contact on a touch-sensitive surface). Contact intensity sensor 165 receives contact intensity information (e.g., pressure information or a proxy for pressure information) from the environment. In some embodiments, at least one contact intensity sensor is collocated with, or proximate to, a touch-sensitive surface (e.g., touch-sensitive display system 112). In some embodiments, at least one contact intensity sensor is located on the back of device 100, opposite touch screen display 112, which is located on the front of device 100.

Device 100 optionally also includes one or more proximity sensors 166. FIG. 1A shows proximity sensor 166 coupled to peripherals interface 118. Alternately, proximity sensor 166 is, optionally, coupled to input controller 160 in I/O subsystem 106. Proximity sensor 166 optionally performs as described in U.S. patent application Ser. No. 11/241,839, “Proximity Detector In Handheld Device”; Ser. No. 11/240,788, “Proximity Detector In Handheld Device”; Ser. No. 11/620,702, “Using Ambient Light Sensor To Augment Proximity Sensor Output”; Ser. No. 11/586,862, “Automated Response To And Sensing Of User Activity In Portable Devices”; and Ser. No. 11/638,251, “Methods And Systems For Automatic Configuration Of Peripherals,” which are hereby incorporated by reference in their entirety. In some embodiments, the proximity sensor turns off and disables touch screen 112 when the multifunction device is placed near the user's ear (e.g., when the user is making a phone call).

Device 100 optionally also includes one or more tactile output generators 167. FIG. 1A shows a tactile output generator coupled to haptic feedback controller 161 in I/O subsystem 106. Tactile output generator 167 optionally includes one or more electroacoustic devices such as speakers or other audio components and/or electromechanical devices that convert energy into linear motion such as a motor, solenoid, electroactive polymer, piezoelectric actuator, electrostatic actuator, or other tactile output generating component (e.g., a component that converts electrical signals into tactile outputs on the device). Contact intensity sensor 165 receives tactile feedback generation instructions from haptic feedback module 133 and generates tactile outputs on device 100 that are capable of being sensed by a user of device 100. In some embodiments, at least one tactile output generator is collocated with, or proximate to, a touch-sensitive surface (e.g., touch-sensitive display system 112) and, optionally, generates a tactile output by moving the touch-sensitive surface vertically (e.g., in/out of a surface of device 100) or laterally (e.g., back and forth in the same plane as a surface of device 100). In some embodiments, at least one tactile output generator sensor is located on the back of device 100, opposite touch screen display 112, which is located on the front of device 100.

Device 100 optionally also includes one or more accelerometers 168. FIG. 1A shows accelerometer 168 coupled to peripherals interface 118. Alternately, accelerometer 168 is, optionally, coupled to an input controller 160 in I/O subsystem 106. Accelerometer 168 optionally performs as described in U.S. Patent Publication No. 20050190059, “Acceleration-based Theft Detection System for Portable Electronic Devices,” and U.S. Patent Publication No. 20060017692, “Methods And Apparatuses For Operating A Portable Device Based On An Accelerometer,” both of which are incorporated by reference herein in their entirety. In some embodiments, information is displayed on the touch screen display in a portrait view or a landscape view based on an analysis of data received from the one or more accelerometers. Device 100 optionally includes, in addition to accelerometer(s) 168, a magnetometer and a GPS (or GLONASS or other global navigation system) receiver for obtaining information concerning the location and orientation (e.g., portrait or landscape) of device 100.

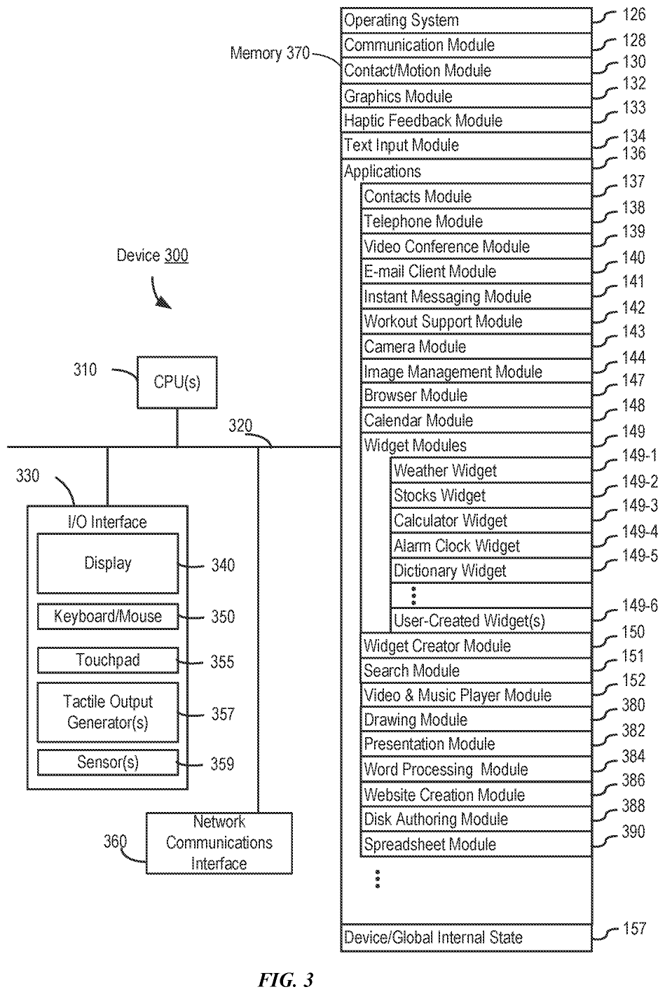

In some embodiments, the software components stored in memory 102 include operating system 126, communication module (or set of instructions) 128, contact/motion module (or set of instructions) 130, graphics module (or set of instructions) 132, text input module (or set of instructions) 134, Global Positioning System (GPS) module (or set of instructions) 135, and applications (or sets of instructions) 136. Furthermore, in some embodiments, memory 102 (FIG. 1A) or 370 (FIG. 3) stores device/global internal state 157, as shown in FIGS. 1A and 3. Device/global internal state 157 includes one or more of: active application state, indicating which applications, if any, are currently active; display state, indicating what applications, views or other information occupy various regions of touch screen display 112; sensor state, including information obtained from the device's various sensors and input control devices 116; and location information concerning the device's location and/or attitude.

Operating system 126 (e.g., Darwin, RTXC, LINUX, UNIX, OS X, iOS, WINDOWS, or an embedded operating system such as VxWorks) includes various software components and/or drivers for controlling and managing general system tasks (e.g., memory management, storage device control, power management, etc.) and facilitates communication between various hardware and software components.

Communication module 128 facilitates communication with other devices over one or more external ports 124 and also includes various software components for handling data received by RF circuitry 108 and/or external port 124. External port 124 (e.g., Universal Serial Bus (USB), FIREWIRE, etc.) is adapted for coupling directly to other devices or indirectly over a network (e.g., the Internet, wireless LAN, etc.). In some embodiments, the external port is a multi-pin (e.g., 30-pin) connector that is the same as, or similar to and/or compatible with, the 30-pin connector used on iPod® (trademark of Apple Inc.) devices.

Contact/motion module 130 optionally detects contact with touch screen 112 (in conjunction with display controller 156) and other touch-sensitive devices (e.g., a touchpad or physical click wheel). Contact/motion module 130 includes various software components for performing various operations related to detection of contact, such as determining if contact has occurred (e.g., detecting a finger-down event), determining an intensity of the contact (e.g., the force or pressure of the contact or a substitute for the force or pressure of the contact), determining if there is movement of the contact and tracking the movement across the touch-sensitive surface (e.g., detecting one or more finger-dragging events), and determining if the contact has ceased (e.g., detecting a finger-up event or a break in contact). Contact/motion module 130 receives contact data from the touch-sensitive surface. Determining movement of the point of contact, which is represented by a series of contact data, optionally includes determining speed (magnitude), velocity (magnitude and direction), and/or an acceleration (a change in magnitude and/or direction) of the point of contact. These operations are, optionally, applied to single contacts (e.g., one finger contacts) or to multiple simultaneous contacts (e.g., “multitouch”/multiple finger contacts). In some embodiments, contact/motion module 130 and display controller 156 detect contact on a touchpad.

In some embodiments, contact/motion module 130 uses a set of one or more intensity thresholds to determine whether an operation has been performed by a user (e.g., to determine whether a user has “clicked” on an icon). In some embodiments, at least a subset of the intensity thresholds are determined in accordance with software parameters (e.g., the intensity thresholds are not determined by the activation thresholds of particular physical actuators and can be adjusted without changing the physical hardware of device 100). For example, a mouse “click” threshold of a trackpad or touch screen display can be set to any of a large range of predefined threshold values without changing the trackpad or touch screen display hardware. Additionally, in some implementations, a user of the device is provided with software settings for adjusting one or more of the set of intensity thresholds (e.g., by adjusting individual intensity thresholds and/or by adjusting a plurality of intensity thresholds at once with a system-level click “intensity” parameter).

Contact/motion module 130 optionally detects a gesture input by a user. Different gestures on the touch-sensitive surface have different contact patterns (e.g., different motions, timings, and/or intensities of detected contacts). Thus, a gesture is, optionally, detected by detecting a particular contact pattern. For example, detecting a finger tap gesture includes detecting a finger-down event followed by detecting a finger-up (liftoff) event at the same position (or substantially the same position) as the finger-down event (e.g., at the position of an icon). As another example, detecting a finger swipe gesture on the touch-sensitive surface includes detecting a finger-down event followed by detecting one or more finger-dragging events, and subsequently followed by detecting a finger-up (liftoff) event.

Graphics module 132 includes various known software components for rendering and displaying graphics on touch screen 112 or other display, including components for changing the visual impact (e.g., brightness, transparency, saturation, contrast, or other visual property) of graphics that are displayed. As used herein, the term “graphics” includes any object that can be displayed to a user, including, without limitation, text, web pages, icons (such as user-interface objects including soft keys), digital images, videos, animations, and the like.

In some embodiments, graphics module 132 stores data representing graphics to be used. Each graphic is, optionally, assigned a corresponding code. Graphics module 132 receives, from applications etc., one or more codes specifying graphics to be displayed along with, if necessary, coordinate data and other graphic property data, and then generates screen image data to output to display controller 156.

Haptic feedback module 133 includes various software components for generating instructions used by tactile output generator(s) 167 to produce tactile outputs at one or more locations on device 100 in response to user interactions with device 100.

Text input module 134, which is, optionally, a component of graphics module 132, provides soft keyboards for entering text in various applications (e.g., contacts 137, e-mail 140, IM 141, browser 147, and any other application that needs text input).

GPS module 135 determines the location of the device and provides this information for use in various applications (e.g., to telephone module 138 for use in location-based dialing; to camera module 143 as picture/video metadata; and to applications that provide location-based services such as weather widgets, local yellow page widgets, and map/navigation widgets).

Applications 136 optionally include the following modules (or sets of instructions), or a subset or superset thereof:
Contacts module 137 (sometimes called an address book or contact list);
Telephone module 138;
Video conference module 139;
E-mail client module 140;
Instant messaging (IM) module 141;
Workout support module 142;
Camera module 143 for still and/or video images;
Image management module 144;
Video player module;
Music player module;
Browser module 147;
Calendar module 148;
Widget modules 149, which optionally include one or more of: weather widget 149-1, stocks widget 149-2, calculator widget 149-3, alarm clock widget 149-4, dictionary widget 149-5, and other widgets obtained by the user, as well as user-created widgets 149-6;
Widget creator module 150 for making user-created widgets 149-6;
Search module 151;
Video and music player module 152, which merges video player module and music player module;
Notes module 153;
Map module 154; and/or
Online video module 155.

Examples of other applications 136 that are, optionally, stored in memory 102 include other word processing applications, other image editing applications, drawing applications, presentation applications, JAVA-enabled applications, encryption, digital rights management, voice recognition, and voice replication.

In conjunction with touch screen 112, display controller 156, contact/motion module 130, graphics module 132, and text input module 134, contacts module 137 are, optionally, used to manage an address book or contact list (e.g., stored in application internal state 192 of contacts module 137 in memory 102 or memory 370), including: adding name(s) to the address book; deleting name(s) from the address book; associating telephone number(s), e-mail address(es), physical address(es) or other information with a name; associating an image with a name; categorizing and sorting names; providing telephone numbers or e-mail addresses to initiate and/or facilitate communications by telephone 138, video conference module 139, e-mail 140, or IM 141; and so forth.

In conjunction with RF circuitry 108, audio circuitry 110, speaker 111, microphone 113, touch screen 112, display controller 156, contact/motion module 130, graphics module 132, and text input module 134, telephone module 138 are optionally, used to enter a sequence of characters corresponding to a telephone number, access one or more telephone numbers in contacts module 137, modify a telephone number that has been entered, dial a respective telephone number, conduct a conversation, and disconnect or hang up when the conversation is completed. As noted above, the wireless communication optionally uses any of a plurality of communications standards, protocols, and technologies.

In conjunction with RF circuitry 108, audio circuitry 110, speaker 111, microphone 113, touch screen 112, display controller 156, optical sensor 164, optical sensor controller 158, contact/motion module 130, graphics module 132, text input module 134, contacts module 137, and telephone module 138, video conference module 139 includes executable instructions to initiate, conduct, and terminate a video conference between a user and one or more other participants in accordance with user instructions.

In conjunction with RF circuitry 108, touch screen 112, display controller 156, contact/motion module 130, graphics module 132, and text input module 134, e-mail client module 140 includes executable instructions to create, send, receive, and manage e-mail in response to user instructions. In conjunction with image management module 144, e-mail client module 140 makes it very easy to create and send e-mails with still or video images taken with camera module 143.

In conjunction with RF circuitry 108, touch screen 112, display controller 156, contact/motion module 130, graphics module 132, and text input module 134, the instant messaging module 141 includes executable instructions to enter a sequence of characters corresponding to an instant message, to modify previously entered characters, to transmit a respective instant message (for example, using a Short Message Service (SMS) or Multimedia Message Service (MMS) protocol for telephony-based instant messages or using XMPP, SIMPLE, or IMPS for Internet-based instant messages), to receive instant messages, and to view received instant messages. In some embodiments, transmitted and/or received instant messages optionally include graphics, photos, audio files, video files and/or other attachments as are supported in an MMS and/or an Enhanced Messaging Service (EMS). As used herein, “instant messaging” refers to both telephony-based messages (e.g., messages sent using SMS or MMS) and Internet-based messages (e.g., messages sent using XMPP, SIMPLE, or IMPS).

In conjunction with RF circuitry 108, touch screen 112, display controller 156, contact/motion module 130, graphics module 132, text input module 134, GPS module 135, map module 154, and music player module, workout support module 142 includes executable instructions to create workouts (e.g., with time, distance, and/or calorie burning goals); communicate with workout sensors (sports devices); receive workout sensor data; calibrate sensors used to monitor a workout; select and play music for a workout; and display, store, and transmit workout data.

In conjunction with touch screen 112, display controller 156, optical sensor(s) 164, optical sensor controller 158, contact/motion module 130, graphics module 132, and image management module 144, camera module 143 includes executable instructions to capture still images or video (including a video stream) and store them into memory 102, modify characteristics of a still image or video, or delete a still image or video from memory 102.

In conjunction with touch screen 112, display controller 156, contact/motion module 130, graphics module 132, text input module 134, and camera module 143, image management module 144 includes executable instructions to arrange, modify (e.g., edit), or otherwise manipulate, label, delete, present (e.g., in a digital slide show or album), and store still and/or video images.

In conjunction with RF circuitry 108, touch screen 112, display controller 156, contact/motion module 130, graphics module 132, and text input module 134, browser module 147 includes executable instructions to browse the Internet in accordance with user instructions, including searching, linking to, receiving, and displaying web pages or portions thereof, as well as attachments and other files linked to web pages.

In conjunction with RF circuitry 108, touch screen 112, display controller 156, contact/motion module 130, graphics module 132, text input module 134, e-mail client module 140, and browser module 147, calendar module 148 includes executable instructions to create, display, modify, and store calendars and data associated with calendars (e.g., calendar entries, to-do lists, etc.) in accordance with user instructions.

In conjunction with RF circuitry 108, touch screen 112, display controller 156, contact/motion module 130, graphics module 132, text input module 134, and browser module 147, widget modules 149 are mini-applications that are, optionally, downloaded and used by a user (e.g., weather widget 149-1, stocks widget 149-2, calculator widget 149-3, alarm clock widget 149-4, and dictionary widget 149-5) or created by the user (e.g., user-created widget 149-6). In some embodiments, a widget includes an HTML (Hypertext Markup Language) file, a CSS (Cascading Style Sheets) file, and a JavaScript file. In some embodiments, a widget includes an XML (Extensible Markup Language) file and a JavaScript file (e.g., Yahoo! Widgets).

In conjunction with RF circuitry 108, touch screen 112, display controller 156, contact/motion module 130, graphics module 132, text input module 134, and browser module 147, the widget creator module 150 are, optionally, used by a user to create widgets (e.g., turning a user-specified portion of a web page into a widget).

In conjunction with touch screen 112, display controller 156, contact/motion module 130, graphics module 132, and text input module 134, search module 151 includes executable instructions to search for text, music, sound, image, video, and/or other files in memory 102 that match one or more search criteria (e.g., one or more user-specified search terms) in accordance with user instructions.

In conjunction with touch screen 112, display controller 156, contact/motion module 130, graphics module 132, audio circuitry 110, speaker 111, RF circuitry 108, and browser module 147, video and music player module 152 includes executable instructions that allow the user to download and play back recorded music and other sound files stored in one or more file formats, such as MP3 or AAC files, and executable instructions to display, present, or otherwise play back videos (e.g., on touch screen 112 or on an external, connected display via external port 124). In some embodiments, device 100 optionally includes the functionality of an MP3 player, such as an iPod (trademark of Apple Inc.).

In conjunction with touch screen 112, display controller 156, contact/motion module 130, graphics module 132, and text input module 134, notes module 153 includes executable instructions to create and manage notes, to-do lists, and the like in accordance with user instructions.

In conjunction with RF circuitry 108, touch screen 112, display controller 156, contact/motion module 130, graphics module 132, text input module 134, GPS module 135, and browser module 147, map module 154 are, optionally, used to receive, display, modify, and store maps and data associated with maps (e.g., driving directions, data on stores and other points of interest at or near a particular location, and other location-based data) in accordance with user instructions.

In conjunction with touch screen 112, display controller 156, contact/motion module 130, graphics module 132, audio circuitry 110, speaker 111, RF circuitry 108, text input module 134, e-mail client module 140, and browser module 147, online video module 155 includes instructions that allow the user to access, browse, receive (e.g., by streaming and/or download), play back (e.g., on the touch screen or on an external, connected display via external port 124), send an e-mail with a link to a particular online video, and otherwise manage online videos in one or more file formats, such as H.264. In some embodiments, instant messaging module 141, rather than e-mail client module 140, is used to send a link to a particular online video. Additional description of the online video application can be found in U.S. Provisional Patent Application No. 60/936,562, “Portable Multifunction Device, Method, and Graphical User Interface for Playing Online Videos,” filed Jun. 20, 2007, and U.S. patent application Ser. No. 11/968,067, “Portable Multifunction Device, Method, and Graphical User Interface for Playing Online Videos,” filed Dec. 31, 2007, the contents of which are hereby incorporated by reference in their entirety.

Each of the above-identified modules and applications corresponds to a set of executable instructions for performing one or more functions described above and the methods described in this application (e.g., the computer-implemented methods and other information processing methods described herein). These modules (e.g., sets of instructions) need not be implemented as separate software programs (such as computer programs (e.g., including instructions)), procedures, or modules, and thus various subsets of these modules are, optionally, combined or otherwise rearranged in various embodiments. For example, video player module is, optionally, combined with music player module into a single module (e.g., video and music player module 152, FIG. 1A). In some embodiments, memory 102 optionally stores a subset of the modules and data structures identified above. Furthermore, memory 102 optionally stores additional modules and data structures not described above.

In some embodiments, device 100 is a device where operation of a predefined set of functions on the device is performed exclusively through a touch screen and/or a touchpad. By using a touch screen and/or a touchpad as the primary input control device for operation of device 100, the number of physical input control devices (such as push buttons, dials, and the like) on device 100 is, optionally, reduced.

The predefined set of functions that are performed exclusively through a touch screen and/or a touchpad optionally include navigation between user interfaces. In some embodiments, the touchpad, when touched by the user, navigates device 100 to a main, home, or root menu from any user interface that is displayed on device 100. In such embodiments, a “menu button” is implemented using a touchpad. In some other embodiments, the menu button is a physical push button or other physical input control device instead of a touchpad.

FIG. 1B is a block diagram illustrating exemplary components for event handling in accordance with some embodiments. In some embodiments, memory 102 (FIG. 1A) or 370 (FIG. 3) includes event sorter 170 (e.g., in operating system 126) and a respective application 136-1 (e.g., any of the aforementioned applications 137-151, 155, 380-390).

Event sorter 170 receives event information and determines the application 136-1 and application view 191 of application 136-1 to which to deliver the event information. Event sorter 170 includes event monitor 171 and event dispatcher module 174. In some embodiments, application 136-1 includes application internal state 192, which indicates the current application view(s) displayed on touch-sensitive display 112 when the application is active or executing. In some embodiments, device/global internal state 157 is used by event sorter 170 to determine which application(s) is (are) currently active, and application internal state 192 is used by event sorter 170 to determine application views 191 to which to deliver event information.

In some embodiments, application internal state 192 includes additional information, such as one or more of: resume information to be used when application 136-1 resumes execution, user interface state information that indicates information being displayed or that is ready for display by application 136-1, a state queue for enabling the user to go back to a prior state or view of application 136-1, and a redo/undo queue of previous actions taken by the user.

Event monitor 171 receives event information from peripherals interface 118. Event information includes information about a sub-event (e.g., a user touch on touch-sensitive display 112, as part of a multi-touch gesture). Peripherals interface 118 transmits information it receives from I/O subsystem 106 or a sensor, such as proximity sensor 166, accelerometer(s) 168, and/or microphone 113 (through audio circuitry 110). Information that peripherals interface 118 receives from I/O subsystem 106 includes information from touch-sensitive display 112 or a touch-sensitive surface.

In some embodiments, event monitor 171 sends requests to the peripherals interface 118 at predetermined intervals. In response, peripherals interface 118 transmits event information. In other embodiments, peripherals interface 118 transmits event information only when there is a significant event (e.g., receiving an input above a predetermined noise threshold and/or for more than a predetermined duration).

In some embodiments, event sorter 170 also includes a hit view determination module 172 and/or an active event recognizer determination module 173.

Hit view determination module 172 provides software procedures for determining where a sub-event has taken place within one or more views when touch-sensitive display 112 displays more than one view. Views are made up of controls and other elements that a user can see on the display.

Another aspect of the user interface associated with an application is a set of views, sometimes herein called application views or user interface windows, in which information is displayed and touch-based gestures occur. The application views (of a respective application) in which a touch is detected optionally correspond to programmatic levels within a programmatic or view hierarchy of the application. For example, the lowest level view in which a touch is detected is, optionally, called the hit view, and the set of events that are recognized as proper inputs are, optionally, determined based, at least in part, on the hit view of the initial touch that begins a touch-based gesture.

Hit view determination module 172 receives information related to sub-events of a touch-based gesture. When an application has multiple views organized in a hierarchy, hit view determination module 172 identifies a hit view as the lowest view in the hierarchy which should handle the sub-event. In most circumstances, the hit view is the lowest level view in which an initiating sub-event occurs (e.g., the first sub-event in the sequence of sub-events that form an event or potential event). Once the hit view is identified by the hit view determination module 172, the hit view typically receives all sub-events related to the same touch or input source for which it was identified as the hit view.

Active event recognizer determination module 173 determines which view or views within a view hierarchy should receive a particular sequence of sub-events. In some embodiments, active event recognizer determination module 173 determines that only the hit view should receive a particular sequence of sub-events. In other embodiments, active event recognizer determination module 173 determines that all views that include the physical location of a sub-event are actively involved views, and therefore determines that all actively involved views should receive a particular sequence of sub-events. In other embodiments, even if touch sub-events were entirely confined to the area associated with one particular view, views higher in the hierarchy would still remain as actively involved views.

Event dispatcher module 174 dispatches the event information to an event recognizer (e.g., event recognizer 180). In embodiments including active event recognizer determination module 173, event dispatcher module 174 delivers the event information to an event recognizer determined by active event recognizer determination module 173. In some embodiments, event dispatcher module 174 stores in an event queue the event information, which is retrieved by a respective event receiver 182.

In some embodiments, operating system 126 includes event sorter 170. Alternatively, application 136-1 includes event sorter 170. In yet other embodiments, event sorter 170 is a stand-alone module, or a part of another module stored in memory 102, such as contact/motion module 130.

In some embodiments, application 136-1 includes a plurality of event handlers 190 and one or more application views 191, each of which includes instructions for handling touch events that occur within a respective view of the application's user interface. Each application view 191 of the application 136-1 includes one or more event recognizers 180. Typically, a respective application view 191 includes a plurality of event recognizers 180. In other embodiments, one or more of event recognizers 180 are part of a separate module, such as a user interface kit or a higher level object from which application 136-1 inherits methods and other properties. In some embodiments, a respective event handler 190 includes one or more of: data updater 176, object updater 177, GUI updater 178, and/or event data 179 received from event sorter 170. Event handler 190 optionally utilizes or calls data updater 176, object updater 177, or GUI updater 178 to update the application internal state 192. Alternatively, one or more of the application views 191 include one or more respective event handlers 190. Also, in some embodiments, one or more of data updater 176, object updater 177, and GUI updater 178 are included in a respective application view 191.

A respective event recognizer 180 receives event information (e.g., event data 179) from event sorter 170 and identifies an event from the event information. Event recognizer 180 includes event receiver 182 and event comparator 184. In some embodiments, event recognizer 180 also includes at least a subset of: metadata 183, and event delivery instructions 188 (which optionally include sub-event delivery instructions).

Event receiver 182 receives event information from event sorter 170. The event information includes information about a sub-event, for example, a touch or a touch movement. Depending on the sub-event, the event information also includes additional information, such as location of the sub-event. When the sub-event concerns motion of a touch, the event information optionally also includes speed and direction of the sub-event. In some embodiments, events include rotation of the device from one orientation to another (e.g., from a portrait orientation to a landscape orientation, or vice versa), and the event information includes corresponding information about the current orientation (also called device attitude) of the device.

Event comparator 184 compares the event information to predefined event or sub-event definitions and, based on the comparison, determines an event or sub-event, or determines or updates the state of an event or sub-event. In some embodiments, event comparator 184 includes event definitions 186. Event definitions 186 contain definitions of events (e.g., predefined sequences of sub-events), for example, event 1 (187-1), event 2 (187-2), and others. In some embodiments, sub-events in an event (e.g., 187-1 and/or 187-2) include, for example, touch begin, touch end, touch movement, touch cancellation, and multiple touching. In one example, the definition for event 1 (187-1) is a double tap on a displayed object. The double tap, for example, comprises a first touch (touch begin) on the displayed object for a predetermined phase, a first liftoff (touch end) for a predetermined phase, a second touch (touch begin) on the displayed object for a predetermined phase, and a second liftoff (touch end) for a predetermined phase. In another example, the definition for event 2 (187-2) is a dragging on a displayed object. The dragging, for example, comprises a touch (or contact) on the displayed object for a predetermined phase, a movement of the touch across touch-sensitive display 112, and liftoff of the touch (touch end). In some embodiments, the event also includes information for one or more associated event handlers 190.

In some embodiments, event definitions 186 include a definition of an event for a respective user-interface object. In some embodiments, event comparator 184 performs a hit test to determine which user-interface object is associated with a sub-event. For example, in an application view in which three user-interface objects are displayed on touch-sensitive display 112, when a touch is detected on touch-sensitive display 112, event comparator 184 performs a hit test to determine which of the three user-interface objects is associated with the touch (sub-event). If each displayed object is associated with a respective event handler 190, the event comparator uses the result of the hit test to determine which event handler 190 should be activated. For example, event comparator 184 selects an event handler associated with the sub-event and the object triggering the hit test.

In some embodiments, the definition for a respective event (187) also includes delayed actions that delay delivery of the event information until after it has been determined whether the sequence of sub-events does or does not correspond to the event recognizer's event type.

When a respective event recognizer 180 determines that the series of sub-events do not match any of the events in event definitions 186, the respective event recognizer 180 enters an event impossible, event failed, or event ended state, after which it disregards subsequent sub-events of the touch-based gesture. In this situation, other event recognizers, if any, that remain active for the hit view continue to track and process sub-events of an ongoing touch-based gesture.

In some embodiments, a respective event recognizer 180 includes metadata 183 with configurable properties, flags, and/or lists that indicate how the event delivery system should perform sub-event delivery to actively involved event recognizers. In some embodiments, metadata 183 includes configurable properties, flags, and/or lists that indicate how event recognizers interact, or are enabled to interact, with one another. In some embodiments, metadata 183 includes configurable properties, flags, and/or lists that indicate whether sub-events are delivered to varying levels in the view or programmatic hierarchy.

In some embodiments, a respective event recognizer 180 activates event handler 190 associated with an event when one or more particular sub-events of an event are recognized. In some embodiments, a respective event recognizer 180 delivers event information associated with the event to event handler 190. Activating an event handler 190 is distinct from sending (and deferred sending) sub-events to a respective hit view. In some embodiments, event recognizer 180 throws a flag associated with the recognized event, and event handler 190 associated with the flag catches the flag and performs a predefined process.

In some embodiments, event delivery instructions 188 include sub-event delivery instructions that deliver event information about a sub-event without activating an event handler. Instead, the sub-event delivery instructions deliver event information to event handlers associated with the series of sub-events or to actively involved views. Event handlers associated with the series of sub-events or with actively involved views receive the event information and perform a predetermined process.

In some embodiments, data updater 176 creates and updates data used in application 136-1. For example, data updater 176 updates the telephone number used in contacts module 137, or stores a video file used in video player module. In some embodiments, object updater 177 creates and updates objects used in application 136-1. For example, object updater 177 creates a new user-interface object or updates the position of a user-interface object. GUI updater 178 updates the GUI. For example, GUI updater 178 prepares display information and sends it to graphics module 132 for display on a touch-sensitive display.

In some embodiments, event handler(s) 190 includes or has access to data updater 176, object updater 177, and GUI updater 178. In some embodiments, data updater 176, object updater 177, and GUI updater 178 are included in a single module of a respective application 136-1 or application view 191. In other embodiments, they are included in two or more software modules.

It shall be understood that the foregoing discussion regarding event handling of user touches on touch-sensitive displays also applies to other forms of user inputs to operate multifunction devices 100 with input devices, not all of which are initiated on touch screens. For example, mouse movement and mouse button presses, optionally coordinated with single or multiple keyboard presses or holds; contact movements such as taps, drags, scrolls, etc. on touchpads; pen stylus inputs; movement of the device; oral instructions; detected eye movements; biometric inputs; and/or any combination thereof are optionally utilized as inputs corresponding to sub-events which define an event to be recognized.

FIG. 2 illustrates a portable multifunction device 100 having a touch screen 112 in accordance with some embodiments. The touch screen optionally displays one or more graphics within user interface (UI) 200. In this embodiment, as well as others described below, a user is enabled to select one or more of the graphics by making a gesture on the graphics, for example, with one or more fingers 202 (not drawn to scale in the figure) or one or more styluses 203 (not drawn to scale in the figure). In some embodiments, selection of one or more graphics occurs when the user breaks contact with the one or more graphics. In some embodiments, the gesture optionally includes one or more taps, one or more swipes (from left to right, right to left, upward and/or downward), and/or a rolling of a finger (from right to left, left to right, upward and/or downward) that has made contact with device 100. In some implementations or circumstances, inadvertent contact with a graphic does not select the graphic. For example, a swipe gesture that sweeps over an application icon optionally does not select the corresponding application when the gesture corresponding to selection is a tap.

Device 100 optionally also include one or more physical buttons, such as “home” or menu button 204. As described previously, menu button 204 is, optionally, used to navigate to any application 136 in a set of applications that are, optionally, executed on device 100. Alternatively, in some embodiments, the menu button is implemented as a soft key in a GUI displayed on touch screen 112.

In some embodiments, device 100 includes touch screen 112, menu button 204, push button 206 for powering the device on/off and locking the device, volume adjustment button(s) 208, subscriber identity module (SIM) card slot 210, headset jack 212, and docking/charging external port 124. Push button 206 is, optionally, used to turn the power on/off on the device by depressing the button and holding the button in the depressed state for a predefined time interval; to lock the device by depressing the button and releasing the button before the predefined time interval has elapsed; and/or to unlock the device or initiate an unlock process. In an alternative embodiment, device 100 also accepts verbal input for activation or deactivation of some functions through microphone 113. Device 100 also, optionally, includes one or more contact intensity sensors 165 for detecting intensity of contacts on touch screen 112 and/or one or more tactile output generators 167 for generating tactile outputs for a user of device 100.

FIG. 3 is a block diagram of an exemplary multifunction device with a display and a touch-sensitive surface in accordance with some embodiments. Device 300 need not be portable. In some embodiments, device 300 is a laptop computer, a desktop computer, a tablet computer, a multimedia player device, a navigation device, an educational device (such as a child's learning toy), a gaming system, or a control device (e.g., a home or industrial controller). Device 300 typically includes one or more processing units (CPUs) 310, one or more network or other communications interfaces 360, memory 370, and one or more communication buses 320 for interconnecting these components. Communication buses 320 optionally include circuitry (sometimes called a chipset) that interconnects and controls communications between system components. Device 300 includes input/output (I/O) interface 330 comprising display 340, which is typically a touch screen display. I/O interface 330 also optionally includes a keyboard and/or mouse (or other pointing device) 350 and touchpad 355, tactile output generator 357 for generating tactile outputs on device 300 (e.g., similar to tactile output generator(s) 167 described above with reference to FIG. 1A), sensors 359 (e.g., optical, acceleration, proximity, touch-sensitive, and/or contact intensity sensors similar to contact intensity sensor(s) 165 described above with reference to FIG. 1A). Memory 370 includes high-speed random access memory, such as DRAM, SRAM, DDR RAM, or other random access solid state memory devices; and optionally includes non-volatile memory, such as one or more magnetic disk storage devices, optical disk storage devices, flash memory devices, or other non-volatile solid state storage devices. Memory 370 optionally includes one or more storage devices remotely located from CPU(s) 310. In some embodiments, memory 370 stores programs, modules, and data structures analogous to the programs, modules, and data structures stored in memory 102 of portable multifunction device 100 (FIG. 1A), or a subset thereof. Furthermore, memory 370 optionally stores additional programs, modules, and data structures not present in memory 102 of portable multifunction device 100. For example, memory 370 of device 300 optionally stores drawing module 380, presentation module 382, word processing module 384, website creation module 386, disk authoring module 388, and/or spreadsheet module 390, while memory 102 of portable multifunction device 100 (FIG. 1A) optionally does not store these modules.

Each of the above-identified elements in FIG. 3 is, optionally, stored in one or more of the previously mentioned memory devices. Each of the above-identified modules corresponds to a set of instructions for performing a function described above. The above-identified modules or computer programs (e.g., sets of instructions or including instructions) need not be implemented as separate software programs (such as computer programs (e.g., including instructions)), procedures, or modules, and thus various subsets of these modules are, optionally, combined or otherwise rearranged in various embodiments. In some embodiments, memory 370 optionally stores a subset of the modules and data structures identified above. Furthermore, memory 370 optionally stores additional modules and data structures not described above.

Attention is now directed towards embodiments of user interfaces that are, optionally, implemented on, for example, portable multifunction device 100.

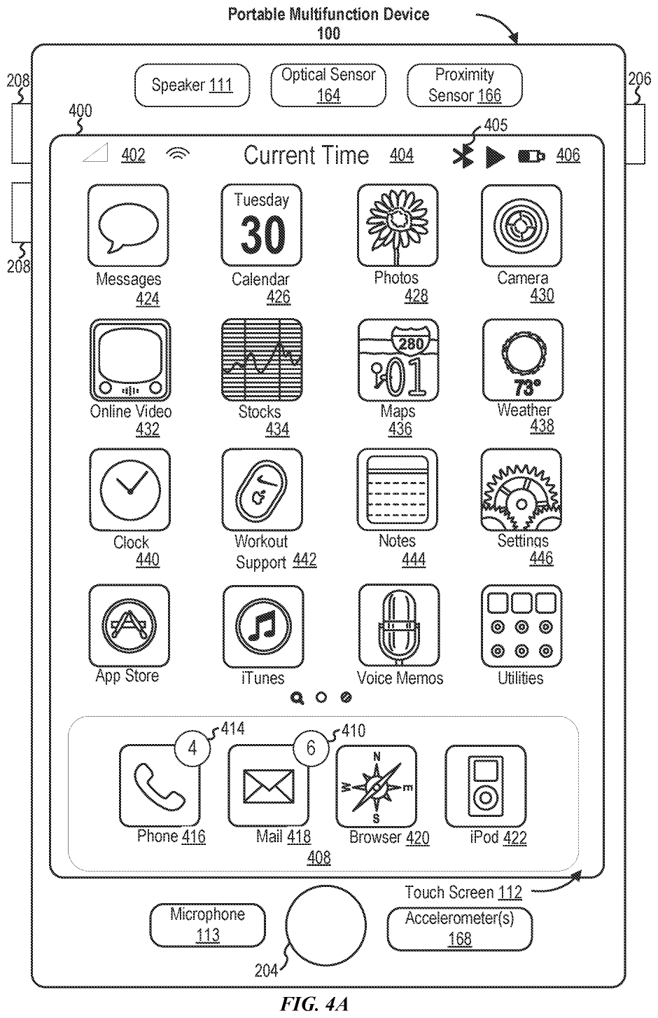

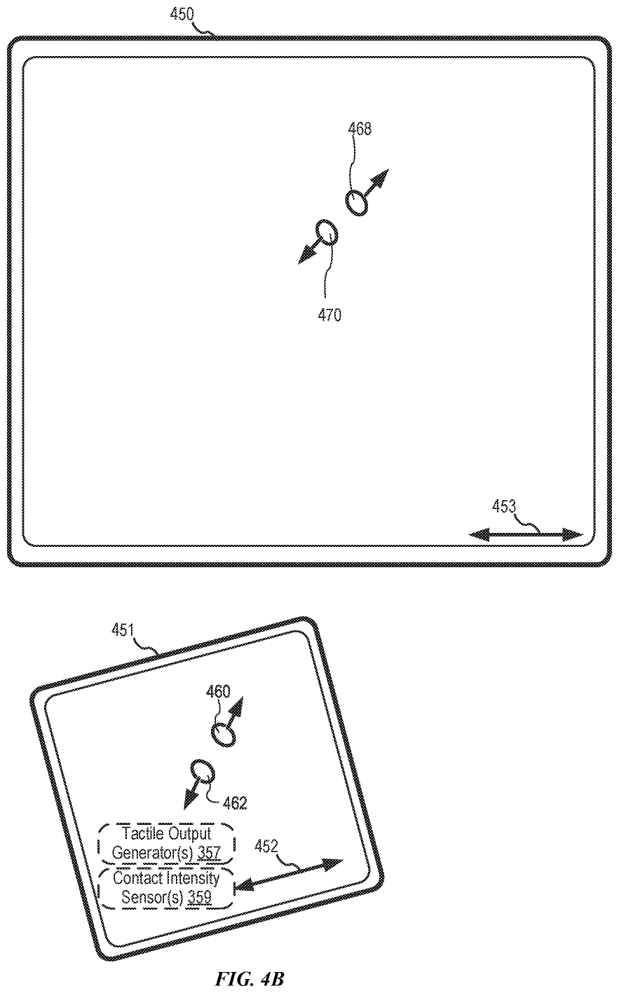

FIG. 4A illustrates an exemplary user interface for a menu of applications on portable multifunction device 100 in accordance with some embodiments. Similar user interfaces are, optionally, implemented on device 300. In some embodiments, user interface 400 includes the following elements, or a subset or superset thereof:
Signal strength indicator(s) 402 for wireless communication(s), such as cellular and Wi-Fi signals;
Time 404;
Bluetooth indicator 405;
Battery status indicator 406;
Tray 408 with icons for frequently used applications, such as:
   Icon 416 for telephone module 138, labeled “Phone,” which optionally includes an indicator 414 of the number of missed calls or voicemail messages;
   Icon 418 for e-mail client module 140, labeled “Mail,” which optionally includes an indicator 410 of the number of unread e-mails;
   Icon 420 for browser module 147, labeled “Browser;” and
   Icon 422 for video and music player module 152, also referred to as iPod (trademark of Apple Inc.) module 152, labeled “iPod;” and
Icons for other applications, such as:
   Icon 424 for IM module 141, labeled “Messages;”
   Icon 426 for calendar module 148, labeled “Calendar;”
   Icon 428 for image management module 144, labeled “Photos;”
   Icon 430 for camera module 143, labeled “Camera;”
   Icon 432 for online video module 155, labeled “Online Video;”
   Icon 434 for stocks widget 149-2, labeled “Stocks;”
   Icon 436 for map module 154, labeled “Maps;”
   Icon 438 for weather widget 149-1, labeled “Weather;”
   Icon 440 for alarm clock widget 149-4, labeled “Clock;”
   Icon 442 for workout support module 142, labeled “Workout Support;”
   Icon 444 for notes module 153, labeled “Notes;” and
   Icon 446 for a settings application or module, labeled “Settings,” which provides access to settings for device 100 and its various applications 136.

It should be noted that the icon labels illustrated in FIG. 4A are merely exemplary. For example, icon 422 for video and music player module 152 is labeled “Music” or “Music Player.” Other labels are, optionally, used for various application icons. In some embodiments, a label for a respective application icon includes a name of an application corresponding to the respective application icon. In some embodiments, a label for a particular application icon is distinct from a name of an application corresponding to the particular application icon.

FIG. 4B illustrates an exemplary user interface on a device (e.g., device 300, FIG. 3) with a touch-sensitive surface 451 (e.g., a tablet or touchpad 355, FIG. 3) that is separate from the display 450 (e.g., touch screen display 112). Device 300 also, optionally, includes one or more contact intensity sensors (e.g., one or more of sensors 359) for detecting intensity of contacts on touch-sensitive surface 451 and/or one or more tactile output generators 357 for generating tactile outputs for a user of device 300.

Although some of the examples that follow will be given with reference to inputs on touch screen display 112 (where the touch-sensitive surface and the display are combined), in some embodiments, the device detects inputs on a touch-sensitive surface that is separate from the display, as shown in FIG. 4B. In some embodiments, the touch-sensitive surface (e.g., 451 in FIG. 4B) has a primary axis (e.g., 452 in FIG. 4B) that corresponds to a primary axis (e.g., 453 in FIG. 4B) on the display (e.g., 450). In accordance with these embodiments, the device detects contacts (e.g., 460 and 462 in FIG. 4B) with the touch-sensitive surface 451 at locations that correspond to respective locations on the display (e.g., in FIG. 4B, 460 corresponds to 468 and 462 corresponds to 470). In this way, user inputs (e.g., contacts 460 and 462, and movements thereof) detected by the device on the touch-sensitive surface (e.g., 451 in FIG. 4B) are used by the device to manipulate the user interface on the display (e.g., 450 in FIG. 4B) of the multifunction device when the touch-sensitive surface is separate from the display. It should be understood that similar methods are, optionally, used for other user interfaces described herein.

Additionally, while the following examples are given primarily with reference to finger inputs (e.g., finger contacts, finger tap gestures, finger swipe gestures), it should be understood that, in some embodiments, one or more of the finger inputs are replaced with input from another input device (e.g., a mouse-based input or stylus input). For example, a swipe gesture is, optionally, replaced with a mouse click (e.g., instead of a contact) followed by movement of the cursor along the path of the swipe (e.g., instead of movement of the contact). As another example, a tap gesture is, optionally, replaced with a mouse click while the cursor is located over the location of the tap gesture (e.g., instead of detection of the contact followed by ceasing to detect the contact). Similarly, when multiple user inputs are simultaneously detected, it should be understood that multiple computer mice are, optionally, used simultaneously, or a mouse and finger contacts are, optionally, used simultaneously.

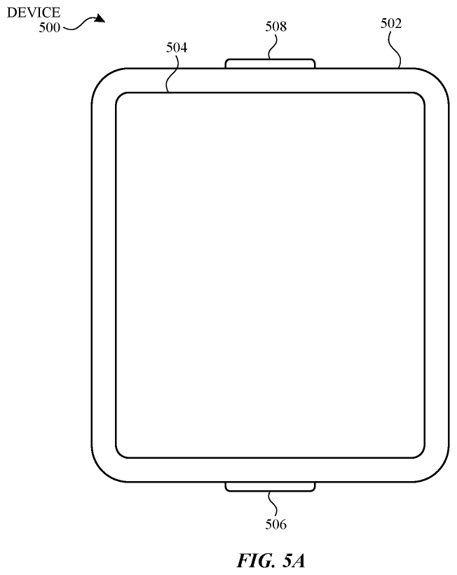

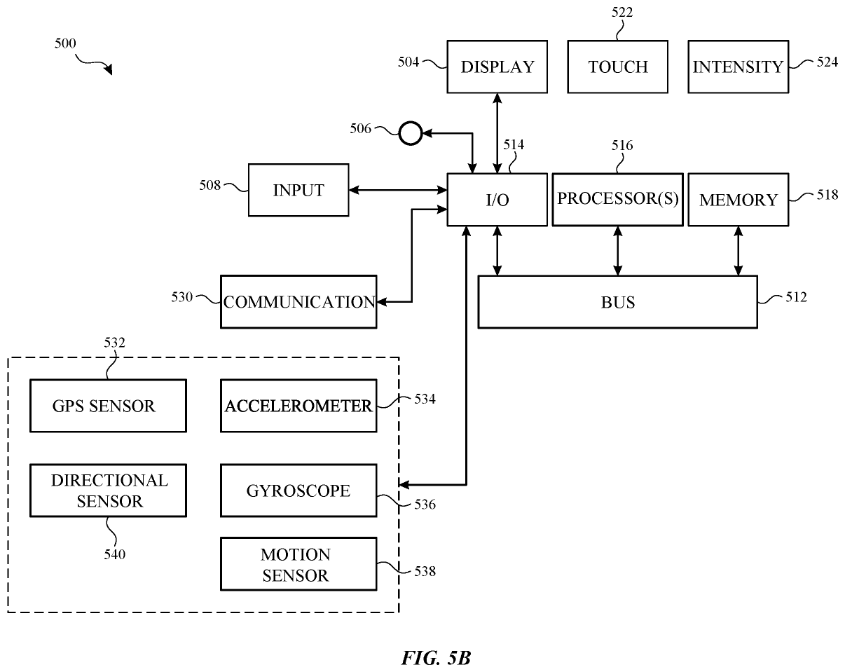

FIG. 5A illustrates exemplary personal electronic device 500. Device 500 includes body 502. In some embodiments, device 500 can include some or all of the features described with respect to devices 100 and 300 (e.g., FIGS. 1A-4B). In some embodiments, device 500 has touch-sensitive display screen 504, hereafter touch screen 504. Alternatively, or in addition to touch screen 504, device 500 has a display and a touch-sensitive surface. As with devices 100 and 300, in some embodiments, touch screen 504 (or the touch-sensitive surface) optionally includes one or more intensity sensors for detecting intensity of contacts (e.g., touches) being applied. The one or more intensity sensors of touch screen 504 (or the touch-sensitive surface) can provide output data that represents the intensity of touches. The user interface of device 500 can respond to touches based on their intensity, meaning that touches of different intensities can invoke different user interface operations on device 500.

Exemplary techniques for detecting and processing touch intensity are found, for example, in related applications: International Patent Application Serial No. PCT/US2013/040061, titled “Device, Method, and Graphical User Interface for Displaying User Interface Objects Corresponding to an Application,” filed May 8, 2013, published as WIPO Publication No. WO/2013/169849, and International Patent Application Serial No. PCT/US2013/069483, titled “Device, Method, and Graphical User Interface for Transitioning Between Touch Input to Display Output Relationships,” filed Nov. 11, 2013, published as WIPO Publication No. WO/2014/105276, each of which is hereby incorporated by reference in their entirety.

In some embodiments, device 500 has one or more input mechanisms 506 and 508. Input mechanisms 506 and 508, if included, can be physical. Examples of physical input mechanisms include push buttons and rotatable mechanisms. In some embodiments, device 500 has one or more attachment mechanisms. Such attachment mechanisms, if included, can permit attachment of device 500 with, for example, hats, eyewear, earrings, necklaces, shirts, jackets, bracelets, watch straps, chains, trousers, belts, shoes, purses, backpacks, and so forth. These attachment mechanisms permit device 500 to be worn by a user.

FIG. 5B depicts exemplary personal electronic device 500. In some embodiments, device 500 can include some or all of the components described with respect to FIGS. 1A, 1B, and 3. Device 500 has bus 512 that operatively couples I/O section 514 with one or more computer processors 516 and memory 518. I/O section 514 can be connected to display 504, which can have touch-sensitive component 522 and, optionally, intensity sensor 524 (e.g., contact intensity sensor). In addition, I/O section 514 can be connected with communication unit 530 for receiving application and operating system data, using Wi-Fi, Bluetooth, near field communication (NFC), cellular, and/or other wireless communication techniques. Device 500 can include input mechanisms 506 and/or 508. Input mechanism 506 is, optionally, a rotatable input device or a depressible and rotatable input device, for example. Input mechanism 508 is, optionally, a button, in some examples.

Input mechanism 508 is, optionally, a microphone, in some examples. Personal electronic device 500 optionally includes various sensors, such as GPS sensor 532, accelerometer 534, directional sensor 540 (e.g., compass), gyroscope 536, motion sensor 538, and/or a combination thereof, all of which can be operatively connected to I/O section 514.

Memory 518 of personal electronic device 500 can include one or more non-transitory computer-readable storage mediums, for storing computer-executable instructions, which, when executed by one or more computer processors 516, for example, can cause the computer processors to perform the techniques described below, including processes 700, 900, 950, and 1100 (FIGS. 7, 9A, 9B, 11). A computer-readable storage medium can be any medium that can tangibly contain or store computer-executable instructions for use by or in connection with the instruction execution system, apparatus, or device. In some examples, the storage medium is a transitory computer-readable storage medium. In some examples, the storage medium is a non-transitory computer-readable storage medium. The non-transitory computer-readable storage medium can include, but is not limited to, magnetic, optical, and/or semiconductor storages. Examples of such storage include magnetic disks, optical discs based on CD, DVD, or Blu-ray technologies, as well as persistent solid-state memory such as flash, solid-state drives, and the like. Personal electronic device 500 is not limited to the components and configuration of FIG. 5B, but can include other or additional components in multiple configurations.

As used here, the term “affordance” refers to a user-interactive graphical user interface object that is, optionally, displayed on the display screen of devices 100, 300, and/or 500 (FIGS. 1A, 3, and 5A-5B). For example, an image (e.g., icon), a button, and text (e.g., hyperlink) each optionally constitute an affordance.

As used herein, the term “focus selector” refers to an input element that indicates a current part of a user interface with which a user is interacting. In some implementations that include a cursor or other location marker, the cursor acts as a “focus selector” so that when an input (e.g., a press input) is detected on a touch-sensitive surface (e.g., touchpad 355 in FIG. 3 or touch-sensitive surface 451 in FIG. 4B) while the cursor is over a particular user interface element (e.g., a button, window, slider, or other user interface element), the particular user interface element is adjusted in accordance with the detected input. In some implementations that include a touch screen display (e.g., touch-sensitive display system 112 in FIG. 1A or touch screen 112 in FIG. 4A) that enables direct interaction with user interface elements on the touch screen display, a detected contact on the touch screen acts as a “focus selector” so that when an input (e.g., a press input by the contact) is detected on the touch screen display at a location of a particular user interface element (e.g., a button, window, slider, or other user interface element), the particular user interface element is adjusted in accordance with the detected input. In some implementations, focus is moved from one region of a user interface to another region of the user interface without corresponding movement of a cursor or movement of a contact on a touch screen display (e.g., by using a tab key or arrow keys to move focus from one button to another button); in these implementations, the focus selector moves in accordance with movement of focus between different regions of the user interface. Without regard to the specific form taken by the focus selector, the focus selector is generally the user interface element (or contact on a touch screen display) that is controlled by the user so as to communicate the user's intended interaction with the user interface (e.g., by indicating, to the device, the element of the user interface with which the user is intending to interact). For example, the location of a focus selector (e.g., a cursor, a contact, or a selection box) over a respective button while a press input is detected on the touch-sensitive surface (e.g., a touchpad or touch screen) will indicate that the user is intending to activate the respective button (as opposed to other user interface elements shown on a display of the device).

As used in the specification and claims, the term “characteristic intensity” of a contact refers to a characteristic of the contact based on one or more intensities of the contact. In some embodiments, the characteristic intensity is based on multiple intensity samples. The characteristic intensity is, optionally, based on a predefined number of intensity samples, or a set of intensity samples collected during a predetermined time period (e.g., 0.05, 0.1, 0.2, 0.5, 1, 2, 5, 10 seconds) relative to a predefined event (e.g., after detecting the contact, prior to detecting liftoff of the contact, before or after detecting a start of movement of the contact, prior to detecting an end of the contact, before or after detecting an increase in intensity of the contact, and/or before or after detecting a decrease in intensity of the contact). A characteristic intensity of a contact is, optionally, based on one or more of: a maximum value of the intensities of the contact, a mean value of the intensities of the contact, an average value of the intensities of the contact, a top 10 percentile value of the intensities of the contact, a value at the half maximum of the intensities of the contact, a value at the 90 percent maximum of the intensities of the contact, or the like. In some embodiments, the duration of the contact is used in determining the characteristic intensity (e.g., when the characteristic intensity is an average of the intensity of the contact over time). In some embodiments, the characteristic intensity is compared to a set of one or more intensity thresholds to determine whether an operation has been performed by a user. For example, the set of one or more intensity thresholds optionally includes a first intensity threshold and a second intensity threshold. In this example, a contact with a characteristic intensity that does not exceed the first threshold results in a first operation, a contact with a characteristic intensity that exceeds the first intensity threshold and does not exceed the second intensity threshold results in a second operation, and a contact with a characteristic intensity that exceeds the second threshold results in a third operation. In some embodiments, a comparison between the characteristic intensity and one or more thresholds is used to determine whether or not to perform one or more operations (e.g., whether to perform a respective operation or forgo performing the respective operation), rather than being used to determine whether to perform a first operation or a second operation.

As used herein, an “installed application” refers to a software application that has been downloaded onto an electronic device (e.g., devices 100, 300, and/or 500) and is ready to be launched (e.g., become opened) on the device. In some embodiments, a downloaded application becomes an installed application by way of an installation program that extracts program portions from a downloaded package and integrates the extracted portions with the operating system of the computer system.

As used herein, the terms “open application” or “executing application” refer to a software application with retained state information (e.g., as part of device/global internal state 157 and/or application internal state 192). An open or executing application is, optionally, any one of the following types of applications:
an active application, which is currently displayed on a display screen of the device that the application is being used on;
a background application (or background processes), which is not currently displayed, but one or more processes for the application are being processed by one or more processors; and
a suspended or hibernated application, which is not running, but has state information that is stored in memory (volatile and non-volatile, respectively) and that can be used to resume execution of the application.

As used herein, the term “closed application” refers to software applications without retained state information (e.g., state information for closed applications is not stored in a memory of the device). Accordingly, closing an application includes stopping and/or removing application processes for the application and removing state information for the application from the memory of the device. Generally, opening a second application while in a first application does not close the first application. When the second application is displayed and the first application ceases to be displayed, the first application becomes a background application.

In some embodiments, the computer system is in a locked state or an unlocked state. In the locked state, the computer system is powered on and operational but is prevented from performing a predefined set of operations in response to user input. The predefined set of operations optionally includes navigation between user interfaces, activation or deactivation of a predefined set of functions, and activation or deactivation of certain applications. The locked state can be used to prevent unintentional or unauthorized use of some functionality of the computer system or activation or deactivation of some functions on the computer system. In some embodiments, in the unlocked state, the computer system is powered on and operational and is not prevented from performing at least a portion of the predefined set of operations that cannot be performed while in the locked state. When the computer system is in the locked state, the computer system is said to be locked. When the computer system is in the unlocked state, the computer is said to be unlocked. In some embodiments, the computer system in the locked state optionally responds to a limited set of user inputs, including input that corresponds to an attempt to transition the computer system to the unlocked state or input that corresponds to powering the computer system off.

Attention is now directed towards embodiments of user interfaces (“UI”) and associated processes that are implemented on an electronic device, such as portable multifunction device 100, device 300, or device 500.

FIGS. 6A-6Q illustrate exemplary user interfaces for tracking body temperature information, in accordance with some embodiments. The user interfaces in these figures are used to illustrate the processes described below, including the processes in FIG. 7.

FIG. 6A illustrates electronic device 600 with touch-sensitive display 602. In FIG. 6A, electronic device 600 displays health summary user interface 604, which displays various sets of health information pertaining to a user (e.g., a user of electronic device 600 and/or a user that is logged into electronic device 600). Health summary user interface 604 includes selectable options 606a-606g. Option 606a displays heart rate information pertaining to the user (e.g., the user's heart rate was measured at 82 bpm at 9:41 am), and is selectable to display additional heart rate information that is not displayed in health summary user interface 604 (e.g., additional and/or earlier heart rate measurements). Option 606b displays body (e.g., wrist) temperature information pertaining to the user, and is selectable to display additional body temperature information that is not displayed in health summary user interface 604. In FIG. 6A, option 606b indicates that wrist data temperature pertaining to the user has not yet been collected and/or there is no wrist temperature data available for the user. Option 606c displays calorie information pertaining to the user (e.g., 1482 calories burned on April 22), and is selectable to display additional calorie information not shown in health summary user interface 604 (e.g., calorie information for previous days). Option 606d displays active calorie information pertaining to the user (e.g., 249 active calories burned on April 22), and is selectable to display additional active calorie information not shown in health summary user interface 604 (e.g., active calorie information for previous days). Option 606e displays steps information pertaining to the user (e.g., 2,945 steps taken on April 22), and is selectable to display additional steps information not shown in health summary user interface 604 (e.g., steps taken on previous and/or other days). Option 606f is selectable to display a user interface through which a user can access additional health information that is not displayed in health summary user interface 604. Option 606g is selectable to initiate a process for modifying the health information that is displayed in health summary user interface 604 (e.g., remove information that is currently included in health summary user interface 604 and/or add other information that is not currently included in health summary user interface 604). Electronic device 600 also displays selectable options 608a-608c. Option 608a is selectable to display health summary user interface 604. Option 608b is selectable to display a health information sharing user interface, in which a user can view health information that the user is sharing with other users (e.g., other users that the user has selected and/or identified) and/or other user's health information that the other users have elected to share with the user of electronic device 600. Option 608c is selectable to browse additional health information pertaining to the user. At FIG. 6A, electronic device 600 detects user input 610 (e.g., a touch input and/or a tap input) corresponding to selection of option 606b.

At FIG. 6B, in response to user input 610, electronic device 600 displays wrist temperature user interface 612. Wrist temperature user interface 612 displays body temperature information (e.g., wrist temperature information) that has been collected from the user. In some embodiments, wrist temperature user interface 612 displays relative body temperature information (e.g., body temperature information relative to a baseline temperature (e.g., x degrees above and/or y degrees below a baseline temperature) rather than absolute body temperature information. Wrist temperature user interface 612 includes options 614a-614c that are selectable to cause body temperature data for different timeframes to be displayed within data chart 614d. For example, option 614a is selectable to view a week of body temperature data within data chart 614d, option 614b is selectable to display a month of body temperature data within data chart 614d, and option 614c is selectable to display six months of body temperature data within data chart 614d. However, in FIG. 6B, there is no body temperature data available for the user (e.g., the user has not yet enabled body temperature logging and/or the user has not taken the appropriate steps for logging body temperature data), so there is no data shown in data chart 614d. Wrist temperature user interface 612 also includes option 614h that is selectable to return to health summary user interface 604, and option 614g that is selectable to initiate a process for manually adding body temperature data.

In FIG. 6B, the user of electronic device 600 has not yet taken the necessary steps to log any body temperature data. For example, the user has not yet enabled the appropriate settings for body temperature data to be collected, and/or the user has not yet taken the appropriate actions required for body temperature data to be collected. Accordingly, electronic device 600 displays, within wrist temperature user interface 612, notification 614e. Notification 614e instructs the user to wear his or her watch to bed, and also instructs the user to enable a sleep tracking function (e.g., a sleep focus state, as will be described in greater detail below) in order to log body temperature information. Electronic device 600 also displays option 614f that is selectable to initiate a process for setting up and/or enabling the sleep tracking function. At FIG. 6B, electronic device 600 detects user input 616 (e.g., a touch input and/or a tap input) corresponding to selection of option 614f.

At FIG. 6C, in response to user input 616, electronic device 600 displays sleep tracking user interface 618. Sleep tracking user interface 618 displays sleep information that has been collected from the user. Sleep tracking user interface 618 includes options 620a-620c that are selectable to cause sleep data for different timeframes to be displayed within data chart 620d. For example, option 620a is selectable to view a week of sleep data within data chart 620d, option 620b is selectable to view a month of sleep data within data chart 620d, and option 620c is selectable to view six months of sleep data within data chart 620d. However, in FIG. 6C, the user has not yet taken the appropriate steps to log sleep data. Accordingly, there is no data shown in data chart 620d. Sleep tracking user interface 618 also includes option 620e that is selectable to display additional sleep data that is not shown in sleep tracking user interface 618, option 620i that is selectable to display health summary user interface 604, and option 620j that is selectable to initiate a process for manually adding and/or logging sleep data.

Sleep tracking user interface 618 includes next sleep schedule information 620f, selectable option 620g, and full sleep schedule information 620h. Next sleep schedule information 620f indicates the next sleep cycle that the user has specified. In FIG. 6C, the user has specified that the user intends to sleep from 11:00 pm that day until 6:00 am the next day. Option 620g is selectable to modify the next sleep cycle (e.g., to specify a start time and/or an end time for the user's next sleep cycle). Full sleep schedule information 620h identifies the full weekly sleep schedule that the user has specified, and is selectable to modify the user's weekly sleep schedule (e.g., to specify, for each day of the week, the user's intended and/or desired sleep start time and sleep end time). In FIG. 6C, the user has defined a sleep schedule that starts at 11:00 μm and ends at 6:00 am every day of the week. In some embodiments, the user is able to specify different sleep start times and different sleep end times for different days (e.g., for different days of the week).

FIG. 6C demonstrated that the user has set up a daily sleep schedule. FIGS. 6D-6G will now demonstrate an example scenario, according to some embodiments, in which the user enables sleep data logging and also enables body temperature information to be measured and logged while the user is sleeping. At FIG. 6D, electronic device 600 displays settings user interface 622. Settings user interface 622 displays various device settings for electronic device 600, including option 624 that is selectable to modify settings for one or more focus states of electronic device 600, which will be described in greater detail below. The one or more focus states includes a sleep focus state, which is also referred to herein as a sleep tracking state. At FIG. 6D, electronic device 600 detects user input 626 (e.g., a touch input and/or a tap input) corresponding to selection of option 624.

At FIG. 6E, in response to user input 626, electronic device 600 displays focus user interface 628. Focus user interface 628 includes options 630a-630d corresponding to different focus states of electronic device 600. Option 630a corresponds to a do not disturb focus state, option 630b corresponds to a sleep focus state, option 630c corresponds to a personal focus state, and option 630d corresponds to a work focus state. Each of these options is selectable to modify one or more settings for the corresponding focus state. In some embodiments, a focus state can be selectively enabled by a user to indicate that the user does not wish to be disturbed while the focus state is enabled. In some embodiments, when a focus state of electronic device 600 is enabled (e.g., any of the four focus states in FIG. 6E), one or more notifications that would normally be displayed and/or otherwise output (e.g., via audio and/or haptic output) are suppressed and are not output by electronic device 600, as will be described in greater detail below. For example, in some embodiments, when a focus state is not enabled, and electronic device 600 receives a message (e.g., a text message) from another user, electronic device 600 displays a notification corresponding to the message. However, when a focus state is enabled, in some embodiments, electronic device 600 does not display notifications corresponding to received messages, as will be described in greater detail below. In some embodiments, different focus states result in different settings being applied. For example, in some embodiments, a first focus state will allow notifications from a certain set of users but a second focus state will not allow notifications from those users. In some embodiments, the sleep focus state will cause suppression of notifications and collection of body temperature information, whereas a different focus state (e.g., do not disturb and/or work) will cause suppression of notifications without collection of body temperature information.

Focus user interface 630e includes toggle 630e. When toggle 630e is toggled to an enabled (or “on”) state, and electronic device 600 enters a particular focus state (e.g., begins operating in the particular focus state) (e.g., automatically and/or based on user input), one or more external devices corresponding to electronic device 600 (e.g., other devices corresponding to the same user) are also caused to enter the particular focus state such that notifications that are suppressed on electronic device 600 during the focus state are also suppressed on other corresponding devices. When toggle 630e is toggled to a disabled (or “off”) state, when electronic device 600 enters a particular focus state and/or operates in the particular focus state, other corresponding devices are not automatically caused to operate in the particular focus state such that even if electronic device 600 suppresses a notification while it is operating in the selected focus state, a different corresponding device is still able to output a notification. Focus user interface 628 also includes option 630f that is selectable to return to settings user interface 622. At FIG. 6E, electronic device 600 detects user input 632 (e.g., a touch input and/or a tap input) corresponding to selection of option 630b.

At FIG. 6F, in response to user input 632, electronic device 600 displays sleep focus user interface 634. Sleep focus user interface 634 includes various options 636a-636h that are selectable to modify one or more settings corresponding to a sleep focus state of electronic device 600. Option 636a is selectable by a user to turn on a sleep focus state (e.g., to cause electronic device 600 to enter and/or operate in the sleep focus state) or to turn off the sleep focus state (e.g., to cause electronic device 600 to exit and/or cease operating in the sleep focus state). In FIG. 6F, option 636a is in the off position, indicating that electronic device 600 is not operating in the sleep focus state (and notifications are not suppressed).

Option 636b is selectable to initiate a process for identifying one or more people (e.g., one or more contacts) that are permitted to disturb the user while the sleep focus state is enabled (e.g., on and/or active). In other words, for a certain subset of people identified by the user, even when electronic device 600 is operating in the sleep focus state, notifications pertaining to those people (e.g., notifications of messages and/or calls received from those people) are not suppressed, whereas notifications pertaining to other people are suppressed. For example, in some embodiments, if a user selects a first contact as being permitted to disturb the user while the sleep focus state is active, when a message is received from the first contact while the sleep focus state is active, electronic device 600 will output a notification (e.g., display a notification, output an audio notification, and/or output haptic notification) as if the sleep focus state was not active. However, notifications pertaining to messages from other users that were not selected by the user will be suppressed because the sleep focus state is active. Similarly, option 636c is selectable to initiate a process for identifying one or more applications that are permitted to disturb the user while the sleep focus state is active (e.g., while electronic device 600 is operating in the sleep focus state).

Option 636d is selectable to open a user interface in which the user can enable or disable a focus status setting. When the focus status setting is enabled, other users (e.g., other users attempting to contact the user of electronic device 600) are able to see an indication when the sleep focus state is active for electronic device 600 (e.g., an indication that electronic device 600 is operating in the sleep focus state and/or has suppressed notifications). When the focus status setting is disabled, other users are not able to see when electronic device 600 is operating in the sleep focus state. Option 636e is selectable to display a user interface in which the user can enable or disable a home screen notifications setting. In some embodiments, when the home screen notifications setting is enabled, notifications that would normally be displayed on a home screen of electronic device 600 (e.g., when the sleep focus state is not active) are also displayed on the home screen of electronic device 600 when the sleep focus state is active. When the home screen notifications setting is disabled, notifications that would normally be displayed on the home screen of electronic device 600 are suppressed (e.g., not displayed) when the sleep focus state is active. Option 636f is selectable to display a user interface in which the user can enable or disable a lock screen notifications setting. In some embodiments, when the lock screen notifications setting is enabled, notifications that would normally be displayed on a lock screen of electronic device 600 (e.g., when the sleep focus state is not active) are also displayed on the lock screen of electronic device 600 when the sleep focus state is active. When the lock screen notifications setting is disabled, notifications that would normally be displayed on the lock screen of electronic device 600 are suppressed (e.g., not displayed) when the sleep focus state is active.

Option 636g is selectable by a user to toggle option 636g between an on state and an off state. When option 636g is in an on state, electronic device 600 automatically enters and/or exits the sleep focus state according to a user-specified sleep schedule (e.g., electronic device 600 automatically enters (e.g., activates) the sleep focus state at a user specified bed time, and electronic device 600 automatically exits (e.g., deactivates) the sleep focus state at a user specified wake time). When option 636g is in an off state, electronic device 600 does not automatically enter and/or exit the sleep focus state according to the user-specified sleep schedule, and the user must manually activate or deactivate the sleep focus state (e.g., using option 636a).

Option 636h is selectable by a user to toggle option 636h between an on state and an off state. When option 636h is in an on state, electronic device 600 collects body temperature information while the user is sleeping (e.g., while electronic device 600 is in the sleep focus state). When option 636h is in an off state, electronic device 600 does not collect body temperature information and/or does not log body temperature information while the user is sleeping (e.g., while electronic device 600 is in the sleep focus state).

Sleep focus user interface 634 also includes option 636i, which displays next sleep schedule information and is selectable to modify next sleep schedule information (similar to next sleep schedule information 620f and option 620g in FIG. 6C), and option 636j, which displays full sleep schedule information and is selectable to modify full sleep schedule information (similar to option 620h in FIG. 6C). Sleep focus user interface 634 also includes options 636k-636m. Option 636k is selectable to display sleep tracking user interface 618. Option 636l is selectable to initiate a process for deleting (e.g., removing) the sleep focus state from electronic device 600. Option 636m is selectable to display focus user interface 628. At FIG. 6F, electronic device 600 detects user input 638a (e.g., a touch input and/or a tap input) corresponding to selection of option 636a, user input 638b (e.g., a touch input and/or a tap input) corresponding to selection of option 636g, and user input 638c (e.g., a touch input and/or a tap input) corresponding to selection of option 636h.

At FIG. 6G, in response to user inputs 638a-638c, options 636a, 636g, and 636h have each been toggled into the “on” position. Option 636a being toggled to the on position indicates that the sleep focus state has been activated and electronic device 600 is now operating in the sleep focus state. Option 636g being toggled to the on position indicates that electronic device 600 will automatically enter and exit the sleep focus state according to the user-specified sleep schedule. Option 636h being toggled to the on position indicates that electronic device 600 will automatically collect body temperature information from the user (e.g., via an external device and/or a wearable device, such as a smart watch or other temperature sensor) when electronic device 600 is operating in the sleep focus state.

At FIG. 6H, electronic device 600 displays lock screen user interface 640. Lock screen user interface 640 includes indication 642a, which indicates that electronic device 600 is in a locked state. Lock screen user interface 640 also includes current time information 642b, current date information 642c, object 642g that is selectable to selectively enable or disable a flashlight, and object 642f that is selectable to display a camera user interface.

In FIG. 6H, electronic device 600 is operating in the sleep focus state. For example, electronic device 600 enters the sleep focus state in response to user input 638a or, in another example, electronic device 600 automatically enters the sleep focus state based on option 636g in FIG. 6G being toggled to an on state and in response to a user-defined sleep schedule. Electronic device 600 displays indication 642d indicating that electronic device 600 is in the sleep focus state. Electronic device 600 also displays alarm indication 642e indicating that the user has not set a wake alarm. In some embodiments, when the user has set a wake alarm, alarm indication 642e displays the time at which the wake alarm will go off.

FIG. 6H also depicts electronic device 800, which is a smart watch with touch-screen display 802, rotatable and depressible input mechanism 804, and button 806. Electronic device 644 displays lock screen user interface 644, indicating that electronic device 800 is in a locked. Lock screen user interface includes current time indication 646a and current date indication 646b. In FIG. 6H, electronic device 800 is also operating in the sleep focus state, as indicated by indication 646d. For example, in some embodiments, as a result of option 630e of FIG. 6D being in the on position, when electronic device 600 enters the sleep focus state, electronic device 800 automatically also enters the sleep focus state. Electronic device 800 also displays alarm indication 646c, similar to alarm indication 642e.

FIGS. 6H-6I illustrate an example embodiment in which, when an electronic device (e.g., electronic device 600 and/or electronic device 800) is in the sleep focus state, additional user inputs are required to transition the electronic device from the locked state to an unlocked state and/or to cease displaying the lock screen user interface compared to when the electronic device is not operating in the sleep focus state.

At FIG. 6H, electronic device 800 detects user input 648, which is a depression (e.g., press) of rotatable and depressible input mechanism 648. In FIG. 6H, electronic device 600 receives biometric information corresponding to the user (e.g., a facial scan and/or facial image) via input device 601 (e.g., a camera and/or depth sensor).

At FIG. 6I, in response to user input 648, electronic device 800 displays user interface 650, which instructs the user to turn rotatable and depressible input mechanism 804 in order to unlock electronic device 800. In some embodiments, when electronic device 800 is not operating in the sleep focus state, user input 648 would be sufficient to unlock electronic device 800. However, because electronic device 800 is operating in the sleep focus state, an additional user input rotating input mechanism 804 is required. Furthermore, in FIG. 6I, in response to receiving biometric information from the user, electronic device 600 displays indication 642a as an unlocked lock, indicating that the biometric information from the user successfully authenticated the user. In some embodiments, when electronic device 600 is in this state, and when electronic device 600 is not operating in the sleep focus state, an upward swipe would be sufficient to cease display of lock screen user interface 640 and unlock electronic device 600. However, in the depicted embodiment, because electronic device 600 is operating in the sleep focus state, additional user input is required to cease display of lock screen user interface. In some embodiments, from the state shown in FIG. 6I, in order to cease display of lock screen user interface 640, a user is first required to press object 642h, and then provide an upward swipe input, and/or the user is required to provide two upward swipe inputs.

At FIG. 6J, electronic device 600 displays health summary user interface 604, which was described above with reference to FIG. 6A. However, now that the user has enabled body temperature logging (e.g., by enabling option 636h in FIG. 6G), option 606b now indicates that electronic device 600 is “collecting data.” At FIG. 6J, electronic device 600 detects user input 652 (e.g., a touch input and/or a tap input) corresponding to selection of option 606b.

At FIG. 6K, in response to user input 652, electronic device 600 displays wrist temperature user interface 612. Now that the user has enabled body temperature logging, wrist temperature user interface 612 no longer displays notification 614e, as was shown in FIG. 6B. However, in FIG. 6K, electronic device 600 has not collected sufficient body temperature data to accurately determine a baseline temperature for the user. As such, wrist temperature user interface 612 still does not display any actual body temperature data in data chart 614d, and displays notification 654 indicating that more body temperature information is needed.

At FIG. 6L, several months have passed from FIG. 6K, and electronic device 600 has collected the threshold amount of body temperature information from the user (e.g., has collected body temperature information for at least five nights and/or for five consecutive nights). Accordingly, wrist temperature user interface 612 now displays user body temperature data in data chart 614d, and no longer displays notification 654. In FIG. 6L, additional options 656a-656c in wrist temperature user interface 612 are shown. In some embodiments, options 656a-656c were part of wrist temperature user interface 612 in the previous figures, but were not shown in those figures. Option 656a is selectable to selectively enable or disable display of body temperature information within health summary user interface 604. Option 656b is selectable to display additional body temperature information that is not shown in wrist temperature user interface 612. Option 656c is selectable to view information pertaining to one or more external devices from which body temperature information for the user was collected (e.g., one or more watches and/or other wearable devices or sensors that were used to collect user body temperature information). At FIG. 6L, electronic device 600 detects user input 658a (e.g., a touch input and/or a tap input) corresponding to selection of option 614b, user input 658b (e.g., a touch input and/or a tap input) corresponding to selection of a data point in data chart 614d, and user input 658c (e.g., a touch input and/or a tap input) corresponding to selection of option 656b.

At FIG. 6M, in response to user input 658b corresponding to selection of a data point in data chart 614d, electronic device 600 displays data box 660. User input 658b corresponded to selection of a data point corresponding to the date Friday, Jun. 24, 2022. In response to user input 658b, electronic device 600 displays data box 660, which includes additional information about the data point (e.g., the user's body temperature on Friday Jun. 24, 2022 was measured to be 0.5 degrees below their baseline temperature). In some embodiments, a user is able to select any of the data points displayed in data chart 614d to view additional information pertaining to the selected data point.

At FIG. 6N, in response to user input 658a corresponding to selection of option 614b, electronic device 600 displays a month of user body temperature data within data chart 614d. Similar to what was shown in FIG. 6M, a user is able to select any data point within data chart 614d to view additional information about the selected data point.

At FIG. 6O, in response to user input 658c corresponding to selection of option 656b, electronic device 600 displays user interface 662. User interface 662 includes representations 664a-664g of a plurality of body temperature information collection instances. For example, representation 664a is representative of a first body temperature information collection instance that occurred on Jun. 25, 2022, and representation 664b is representative of a second body temperature information collection instance that occurred on Jun. 24, 2022, and so forth. In some embodiments, user interface 662 includes representations of all instances in which body temperature information was collected from the user, or representations of all body temperature information collection instances that occurred within a predetermined period of time (e.g., in the current calendar year, within the last month, within the last three months, within the last six months, within the last year, within the last three years, etc.). User interface 662 also includes option 664h that is selectable to return to wrist temperature user interface 612. At FIG. 6O, electronic device 600 detects user input 666 (e.g., a tap input and/or a touch input) corresponding to selection of representation 664a.

At FIG. 6P, in response to user input 666, electronic device 600 displays user interface 668. User interface 668 displays additional information pertaining to the selected body temperature information collection instance. For example, in the depicted embodiment, user interface 668 includes start time information 670b (e.g., the time at which body temperature information collection begin for this particular collection instance), end time information 670c (e.g., the time at which body temperature information collection ended for this particular collection instance), and data addition information 670e (e.g., the date and time on which the body temperature information was received by electronic device 600 from an external device). User interface 668 also includes source information 670d identifying the external device which collected the body temperature information and/or the external device from which the body temperature information was received. In some embodiments, electronic device 600 aggregates body temperature information collected by and/or received from a plurality of external devices. In some embodiments, wrist temperature user interface 612 displays body temperature information collected by and/or received from a plurality of external devices. User interface 668 also includes surface temperature information 670f and wrist temperature information 670a. As discussed above, wrist temperature user interface 612 displays baseline temperature information, and does not display actual temperature measurements (e.g., the actual measured temperature). However, user interface 668 displays surface temperature information 670f, which indicates the actual temperature that was measured at the surface of the user's skin, and wrist temperature information 670a, which indicates the user's body temperature as derived from surface temperature information 670f.

In FIG. 6Q, electronic device 600 displays wrist temperature user interface 612. However, in FIG. 6Q, the user has not been consistently wearing his or her wearable device to sleep and, as such, electronic device 600 has not been able to consistently collect body temperature information while the user is sleeping. Accordingly, electronic device 600 has determined that electronic device 600 does not have sufficient data to continue accurately displaying body temperature information (e.g., electronic device 600 has collected less than a threshold amount of body temperature information during a predetermined period of time (e.g., in the last week, in the last month, etc.)). In response to this determination, electronic device 600 ceases displaying body temperature information in data chart 614d, and displays indication 672, which informs the user that electronic device 600 does not have sufficient body temperature information, and instructs the user to consistently wear his or her wearable device to sleep.

FIG. 7 is a flow diagram illustrating a method for tracking body temperature information using a computer system in accordance with some embodiments. Method 700 is performed at a computer system (e.g., 100, 300, 500) (e.g., a wearable device, a smart watch, a smart phone, a tablet, and/or a computer system controlling an external display) (e.g., 600, 800) that is in communication with a display generation component (e.g., a display controller, a touch-sensitive display system; and/or a display (e.g., integrated and/or connected)) (e.g., 602, 802), one or more input devices (e.g., a touch-sensitive surface (e.g., a touch-sensitive display); an accelerometer; a rotatable input mechanism; a depressible input mechanism; and/or a rotatable and depressible input mechanism) (e.g., 601, 602, 802, 804, 806), and a temperature sensor (e.g., an integrated sensor; a sensor integrated into an external device that is in communication with the computer system) (e.g., a temperature sensor integrated into device 800). Some operations in method 700 are, optionally, combined, the orders of some operations are, optionally, changed, and some operations are, optionally, omitted.

As described below, method 700 provides an intuitive way for tracking body temperature information. The method reduces the cognitive burden on a user for tracking body temperature information, thereby creating a more efficient human-machine interface. For battery-operated computing devices, enabling a user to track body temperature information faster and more efficiently conserves power and increases the time between battery charges.

The computer system (e.g., 600, 800) receives (702), via the one or more input devices (e.g., 601, 602, 802, 804, 806), an input (e.g., a first set of user inputs and/or one or more user inputs) (e.g., one or more touch inputs, one or more non-touch inputs, and/or one or more gestures) corresponding to a request to enter a sleep tracking mode (e.g., 638a and/or 638b and/or one or more user inputs defining a sleep schedule (e.g., sleep schedule shown in 636i, 636j)) (e.g., a mode and/or state in which one or more notifications (e.g., notifications of a first type) received by the computer system are suppressed and/or in which information pertaining to the sleep (e.g., sleep quality) of a user is collected). In response to the input (704), the computer system enters the sleep tracking mode (e.g., enables the sleep tracking mode and/or causes the computer system to operate in the sleep tracking). In some embodiments, entering the sleep tracking mode includes initiating a process to collect user body temperature information. During the sleep tracking mode (706) (e.g., while the computer system is in the sleep tracking mode), the computer system causes a first set of user body temperature information (e.g., user body temperature information displayed in data chart 614d) (e.g., one or more user body temperature measurements) to be collected via the temperature sensor (e.g., a temperature sensor integrated into electronic device 800).

The computer system receives (708) notification data of a first type (e.g., notifications of a type that are subject to being suppressed while in a sleep tracking mode) (in some embodiments, all notifications are suppressed in the sleep tracking mode) (in some embodiments, notifications of a second type (e.g., emergency notifications, wake alarm notifications), are not suppressed while in the sleep tracking mode). In response to receiving the notification data of the first type (710): in accordance with a determination that the computer system is not in the sleep tracking mode (712), the computer system outputs a first notification (e.g., displays a first notification; causes the display generation component to transition from an inactive state (e.g., off and/or a state in which content is not displayed on the display generation component) to an active state (e.g., on and/or a state in which content is displayed); outputs an audio output; and/or outputs a haptic output) corresponding to the notification data of the first type; and in accordance with a determination that the computer system is currently in the sleep tracking mode (714) (e.g., FIGS. 6H-6I) (e.g., that the computer system is in the sleep tracking mode when the notification data of the first type is received), the computer system forgoes outputting (e.g., suppresses) the first notification corresponding to the notification data of the first type (e.g., forgoes displaying the first notification; forgoes causing the display generation component to transition from an inactive state to an active state; forgoes outputting an audio output; and/or forgoes outputting a haptic output).

After causing the first set of user body temperature information to be collected (716), the computer system displays, via display generation component (e.g., 602), a body temperature user interface (e.g., 612) that includes representations of one or more sets of user body temperature information (e.g., body temperature information displayed in data chart 614d in FIG. 6L) (e.g., user body temperature information associated with a user of the computer system, collected from the user of the computer system and/or collected by the computer system) including the first set of user body temperature information (e.g., a body temperature user interface that depicts representations of a user's body temperature (e.g., depicts and/or demonstrates changes in the user's body temperature) over a period of time).

Automatically collecting the first set of user body temperature information while the device is in the sleep tracking mode allows for collection of user body temperature information with fewer user inputs, thereby reducing the number of user inputs required to perform an operation. Collecting user body temperature information during a sleep tracking mode that is initiated by a user input reduces the risk that body temperature information is erroneously collected during a non-sleep state (e.g., due to a false positive on a sleep state determination); doing so improves the quality of the data and improves operations performed by the computer system based on body temperature data that is attributed to a sleep state, which enhances the operability of the system and makes the user-system interface more efficient (e.g., by helping the user to provide proper inputs and reducing errors) which, additionally, reduces power usage and improves battery life of the device by enabling the user to use the system more quickly and efficiently

In some embodiments, the computer system receives, via the one or more input devices, a second input (e.g., a second set of user inputs and/or one or more user inputs) (e.g., one or more touch inputs, one or more non-touch inputs, and/or one or more gestures) corresponding to a request to enter a first silent mode (e.g., do not disturb, personal focus state, and/or work focus state in FIG. 6E) (e.g., a mode and/or state in which one or more notifications (e.g., notifications of a first type) received by the computer system are suppressed and, in some embodiments, a mode and/or state in which information pertaining to the sleep (e.g., sleep quality) of a user is not collected) different from the sleep tracking mode (e.g., sleep focus state in FIG. 6E). In some embodiments, in response to receiving the second input, the computer system enters the first silent mode (enabling the first silent mode and/or causing the computer system to operate in the first silent mode) (in some embodiments, without entering the sleep tracking mode) without initiating a process to collect body temperature information. In some embodiments, the computer system collects body temperature information while forgoing storing body temperature information and/or collects body temperature information of a second type while forgoing collecting and/or storing body temperature information of a third type.

In some embodiments, the computer system receives second notification data of the first type. In some embodiments, in response to receiving the second notification data of the first type: in accordance with a determination that the computer system is not in the first silent mode (e.g., the do not disturb state, the personal focus state, and/or the work focus state in FIG. 6E are not active and/or enabled) and that the computer system is not in the sleep tracking mode (e.g., the sleep focus state in FIG. 6E is not active and/or enabled (e.g., option 636a is not enabled), the computer system outputs a second notification (e.g., displays a second notification; causes the display generation component to transition from an inactive state (e.g., off and/or a state in which content is not displayed on the display generation component) to an active state (e.g., on and/or a state in which content is displayed); outputs an audio output; and/or outputs a haptic output) corresponding to the second notification data of the first type; and in accordance with a determination that the computer system is currently in the first silent mode (e.g., at least one of the do not disturb state, the personal focus state, and/or the work focus state in FIG. 6E is active and/or enabled) (e.g., that the computer system is in the first silent mode when the second notification data of the first type is received), the computer system forgoes outputting (e.g., suppresses) a notification corresponding to the second notification data of the first type (e.g., forgoes displaying the second notification; forgoes causing the display generation component to transition from an inactive state to an active state; forgoes outputting an audio output; and/or forgoes outputting a haptic output). Automatically suppressing notifications when the computer system is in the first silent mode allows a user to perform this operation with fewer user inputs, thereby reducing the number of user inputs required to perform an operation. Furthermore, automatically collecting temperature information when the computer system is in the sleep tracking mode, and forgoing collecting temperature information when the computer system is in the first silent mode, allows a user to perform these operations with fewer user inputs.

In some embodiments, the computer system receives, via the one or more input devices, a third input (e.g., a third set of user inputs and/or one or more user inputs) (e.g., one or more touch inputs, one or more non-touch inputs, and/or one or more gestures) corresponding to a request to define a sleep schedule (e.g., sleep schedule shown in FIG. 6C (e.g., 620f, 620h) and/or FIG. 6F (e.g., 636i, 636j), wherein the sleep schedule includes, for at least a first day (e.g., a first day of the week (e.g., Sunday, Monday, Tuesday, etc.) and/or a first calendar date), a first sleep start time and a first sleep end time. In some embodiments, the sleep schedule includes, for a second day (e.g., a second day of the week and/or a second calendar date) different from the first day, a second sleep start time and a second sleep end time. Subsequent to receiving the third input: in accordance with a determination that a current time corresponds to the first sleep start time (e.g., is the first sleep start time), the computer system enters the sleep tracking mode (e.g., sleep focus state in FIGS. 6H-6I); and in accordance with a determination that the current time does not correspond to the first sleep start time (e.g., is not the first sleep start time), the computer system forgoes entering the sleep tracking mode (e.g., maintains the computer system in a non-sleep tracking mode and/or maintains the computer system in the sleep tracking mode). In some embodiments, the sleep schedule defines sleep start times and sleep end times for a plurality of days. In some embodiments, the sleep start times and/or the sleep end times are the same for two or more days of the plurality of days. In some embodiments, the sleep start times and/or the sleep end times are different for two or more days of the plurality of days. In some embodiments, subsequent to receiving the third input, and in accordance with a determination that a current time corresponds to the first sleep end time (e.g., is the sleep end time), the computer system exits the sleep tracking mode (e.g., transitions the computer system from the sleep tracking mode to a non-sleep tracking mode). Automatically engaging a sleep tracking mode based on a user-defined schedule allows the user to enable the sleep tracking mode with fewer user inputs, thereby reducing the number of user inputs required to perform an operation.

In some embodiments, causing the first set of user body temperature information (e.g., user body temperature information shown in chart 614d) to be collected includes: causing a first external device (e.g., 800) (e.g., a wearable device, a smart watch, a smart phone, a tablet, and/or a computer system controlling an external display) separate from the computer system (e.g., 600) to collect the first set of user body temperature information; and receiving the first set of user body temperature information from the first external device (e.g., via wireless and/or wired transmission) (e.g., directly or indirectly). Automatically causing a first external device to collect the first set of user body temperature information while the computer system is in the sleep tracking mode allows for collection of user body temperature information with fewer user inputs, thereby reducing the number of user inputs required to perform an operation.

In some embodiments, prior to receiving the input, the computer system displays, via the display generation component, the body temperature user interface (e.g., 612), including: in accordance with a determination that a threshold amount of body temperature information (e.g., user body temperature information associated with a user of the computer system, collected from the user of the computer system and/or collected by the computer system) has not been collected (e.g., FIG. 6K) (e.g., less than a threshold number of days of body temperature information has been collected and/or less than a threshold number of hours of body temperature information has been collected), displaying an indication that the threshold amount of body temperature information has not been collected (e.g., data chart 614d in FIG. 6K and/or 654) (and, in some embodiments, forgoing displaying representations of one or more sets of user body temperature information (e.g., collected from the user and/or by the computer system)); and in accordance with a determination that the threshold amount of body temperature information has been collected (e.g., FIG. 6L), displaying representations of one or more sets of user body temperature information (e.g., body temperature information displayed in data chart 614d in FIG. 6L) (e.g., user body temperature information associated with a user of the computer system, collected from the user and/or collected by the computer system) without displaying the indication that the threshold amount of body temperature information has not been collected (e.g., 654). Displaying the indication that the threshold amount of body temperature information has not been collected provides the user with visual feedback about the state of the system (e.g., that the system has not yet collected the threshold amount of body temperature information), thereby providing improved visual feedback to the user.

In some embodiments, subsequent to displaying the body temperature user interface (e.g., 612) that includes the representations of one or more sets of user body temperature information (e.g., 612 in FIG. 6L), the computer system displays a second instance of the body temperature user interface (e.g., 612), wherein: in accordance with a determination that a second set of criteria are satisfied (e.g., in accordance with a determination that body temperature collection frequency criteria are satisfied (e.g., a threshold amount of body temperature information has been collected in a defined period of time (e.g., the immediately preceding week, month, three months, six months, and/or year))), the second instance of the body temperature user interface includes representations of a second set of user body temperature information (e.g., an updated version of FIG. 6L that includes updated user body temperature information) (e.g., a set of user body temperature information that is collected after the first set of user body temperature information); and in accordance with a determination that the second set of criteria are not satisfied (e.g., in accordance with a determination that body temperature collection frequency criteria are not satisfied (e.g., a threshold amount of body temperature information has not been collected in a defined period of time (e.g., the immediately preceding week, month, three months, six months, and/or year))), the second instance of the body temperature user interface does not include the representations of the second set of user body temperature information (e.g., user interface 612 displaying data chart 614d in FIG. 6Q) (and, in some embodiments, does not include representations of user body temperature information) and includes a second indication indicating that the second set of criteria are not satisfied (e.g., data chart 614d in FIG. 6Q and/or 672) (e.g., indicating that collection of body temperature information is too sporadic and/or infrequent). Displaying the indication that the second set of criteria are not satisfied provides the user with visual feedback about the state of the system (e.g., that the system has determined that the second set of criteria are not satisfied), thereby providing improved visual feedback to the user.

In some embodiments, while displaying the body temperature user interface (e.g., 612), the computer system receives, via the one or more input devices, a selection input (e.g., 658a) (e.g., one or more touch inputs, one or more non-touch inputs, and/or one or more gestures). In response to receiving the selection input: in accordance with a determination that the selection input corresponds to selection of a first timeframe option (e.g., 614a, 614b, 614c) corresponding to a first timeframe (e.g., a first duration of time (e.g., a week, a month, six months, and/or a year)), the computer system displays, within the body temperature user interface, representations of one or more sets of user body temperature information corresponding to the first timeframe (e.g., in FIG. 6L user interface 612 displays body temperature information for a weekly timeframe, in FIG. 6N, user interface 612 displays body temperature information for a monthly timeframe) (e.g., collected during the first timeframe and/or collected during the first duration of time corresponding to the first timeframe option) (and, optionally, without displaying representations of user body temperature information collected outside of the first timeframe and/or outside of the first duration of time corresponding to the first timeframe option); and in accordance with a determination that the selection input corresponds to selection of a second timeframe option (e.g., 614a, 614b, 614c) different from the first timeframe option, wherein the second timeframe option corresponds to a second timeframe (e.g., a second duration of time (e.g., a week, a month, six months, and/or a year)) different from the first timeframe, the computer system displays, within the body temperature user interface, representations of one or more sets of user body temperature information corresponding to the second timeframe (e.g., in FIG. 6L user interface 612 displays body temperature information for a weekly timeframe, in FIG. 6N, user interface 612 displays body temperature information for a monthly timeframe) (e.g., collected during the second timeframe and/or collected during the second duration of time corresponding to the second timeframe option) (and, optionally, without displaying representations of user body temperature information collected outside of the second timeframe and/or outside of the second duration of time corresponding to the second timeframe option). Providing different selectable options corresponding to different timeframes that allows a user to display body temperature information corresponding to different timeframes allows for a user to view different sets of body temperature information with fewer user inputs, thereby reducing the number of user inputs required to perform an operation.

In some embodiments, the representations of one or more sets of user body temperature information includes one or more relative temperature measurements (e.g., data chart 614 in FIGS. 6L, 6M, and 6N displays relative temperature measurements (e.g., +2, +1, 0, −1, −2)) (e.g., relative temperature measurements indicating the number of degrees above and/or below a baseline temperature) (and, optionally, does not include absolute temperature measurements). Automatically collecting the first set of user body temperature information while the device is in the sleep tracking mode allows for collection of user body temperature information with fewer user inputs, thereby reducing the number of user inputs required to perform an operation.

In some embodiments, the computer system displays, via the display generation component, a first details user interface (e.g., 668) corresponding to the first set of user body temperature information, wherein the first details user interface includes information about the first set of user body temperature information (e.g., 670a-670f) that is not displayed in the body temperature user interface (e.g., collection start time, collection end time, actual body temperature measurement information (whereas, in some embodiments, the body temperature user interface displays relative body temperature measurement information), and/or external device information (e.g., information identifying the name, make, and/or model of an external device used to collect the first set of user body temperature information)). In some embodiments, the computer system displays a second details user interface corresponding to a second set of user body temperature information different from the first set of user body temperature information, wherein the second details user interface includes information about the second set of user body temperature information that is not displayed in the body temperature user interface. Displaying a first details user interface provides the user with visual feedback about the state of the system (e.g., that the system has collected additional information about the first set of user body temperature information that is not included in the body temperature user interface), thereby providing improved visual feedback to the user. Providing the first details user interface in which a user can view additional details about the first set of user body temperature information allows the user to perform this operation with fewer user inputs.

In some embodiments, the first details user interface (e.g., 668) includes: a start time (e.g., 670b) corresponding to the first set of user body temperature information (e.g., a time and/or a date on which collection of the first set of user body temperature information started); and an end time (e.g., 670c) corresponding to the first set of user body temperature information (e.g., a time and/or a date on which collection of the first set of user body temperature information ended). Displaying a first details user interface provides the user with visual feedback about the state of the system (e.g., that the system has collected additional information about the first set of user body temperature information that is not included in the body temperature user interface), thereby providing improved visual feedback to the user. Providing the first details user interface in which a user can view additional details about the first set of user body temperature information allows the user to perform this operation with fewer user inputs.

In some embodiments, the body temperature user interface (e.g., 612) includes relative body temperature information corresponding to the first set of user body temperature information (e.g., relative temperature measurements indicating the number of degrees above and/or below a baseline temperature) (and, optionally, does not include absolute temperature measurements); and the first details user interface (e.g., 668) includes one or more absolute body temperature measurements (e.g., 670a, 670f) corresponding to the first set of user body temperature information (e.g., actual measured body temperature and/or measurements indicative of the actual body temperature of the user). Displaying a first details user interface provides the user with visual feedback about the state of the system (e.g., that the system has collected additional information about the first set of user body temperature information that is not included in the body temperature user interface), thereby providing improved visual feedback to the user. Providing the first details user interface in which a user can view additional details about the first set of user body temperature information allows the user to perform this operation with fewer user inputs.

In some embodiments, the first details user interface (e.g., 668) includes external device information (e.g., 670d) corresponding to the first set of user body temperature information (e.g., information pertaining to an external device (e.g., separate from the computer system) that was used to collect the first set of user body temperature information (e.g., name, model, and/or other identifier of external device)). Displaying a first details user interface provides the user with visual feedback about the state of the system (e.g., that the system has collected additional information about the first set of user body temperature information that is not included in the body temperature user interface), thereby providing improved visual feedback to the user. Providing the first details user interface in which a user can view additional details about the first set of user body temperature information allows the user to perform this operation with fewer user inputs.

In some embodiments, the computer system (e.g., 600) receives first notification data corresponding to one or more messages received from a first external user (e.g., a first external user different from a user of the computer system and/or a first external user of a first external device separate from the computer system) and/or one or more messages received from a first application (e.g., a first application running on the computer system and/or a first application running on an external device separate from the computer system). In response to receiving the first notification data: in accordance with a determination that the computer system is not in the sleep tracking mode (e.g., option 636a is in the inactive and/or off state) (e.g., when the first notification data is received), the computer system outputs a second notification (e.g., displays a second notification; causes the display generation component to transition from an inactive state (e.g., off and/or a state in which content is not displayed on the display generation component) to an active state (e.g., on and/or a state in which content is displayed); outputs an audio output; and/or outputs a haptic output) corresponding to the first notification data; and in accordance with a determination that the computer system is currently in the sleep tracking mode (e.g., option 636a is in the active and/or on state) (e.g., when the first notification data is received), and that the first notification data comprises notification data of a second type (e.g., notification data that is suppressed while the computer system is in the sleep tracking mode, notification data corresponding to one or more messages received from a first subset of external users (e.g., users that are not identified in object 636b in FIG. 6F) and/or notification data corresponding to one or more messages received from a first subset of applications (e.g., applications that are not identified in object 636c in FIG. 6F)), the computer system forgoes outputting (e.g., suppresses) the second notification corresponding to the first notification data (e.g., forgoes displaying the second notification; forgoes causing the display generation component to transition from an inactive state to an active state; forgoes outputting an audio output; and/or forgoes outputting a haptic output). Automatically suppressing notifications pertaining to messages received from other users or applications when the computer system is in the sleep tracking mode allows a user to perform these operations with fewer user inputs.

In some embodiments, in response to receiving the first notification data: in accordance with a determination that the computer system is currently in the sleep tracking mode (e.g., option 636a is in the active and/or on state), and that the first notification data comprises notification data of a third type (e.g., notification data that is not suppressed while the computer system is in the sleep tracking mode, notification data corresponding to one or more messages received from a second subset of external users (e.g., a preapproved and/or preselected subset of external users) (e.g., users identified in object 636b in FIG. 6F) different from the first subset and/or corresponding to one or more messages receives from a second subset of applications (e.g., a preapproved and/or preselected subset of external users) (e.g., applications identified in object 636c in FIG. 6F) different from the first subset) different from the second type, the computer system outputs the second notification corresponding to the first notification data. Automatically suppressing notifications pertaining to messages received from certain unapproved users or applications when the computer system is in the sleep tracking mode, while allowing notifications pertaining to messages received from preapproved users or applications when the computer system is in the sleep tracking mode, allows a user to perform these operations with fewer user inputs.

In some embodiments, during the sleep tracking mode (e.g., while the computer system is in the sleep tracking mode): in accordance with a determination that the computer system is in the sleep tracking mode (e.g., option 636a is in the active and/or on state), the computer system causes a second external device (e.g., 1070) (e.g., a second external device corresponding to a second user different from a user of the computer system) different from the computer system (e.g., separate from the computer system) to display an indication that the computer system has suppressed notifications (e.g., has suppressed one or more types of notifications) (e.g., based on option 636d being in the activated and/or engaged state). In some embodiments, while the computer system is not in the sleep tracking mode, and in accordance with a determination that the computer system is not in the sleep tracking mode, the computer system forgoes causing and/or does not cause the second external device to display the indication that the computer system is in the sleep tracking mode. Causing the second external device to display the indication that the computer system has suppressed notifications provides the user of the second external device with visual feedback about the state of the computer system (e.g., that the computer system has suppressed notifications), thereby providing improved visual feedback to the user.

In some embodiments, the representations of one or more sets of user body temperature information (e.g., body temperature information shown in data chart 614d) includes: a representation of a third set of user body temperature information (e.g., a third period of time during which body temperature information was collected from the user) collected by a second external device (e.g., a wearable device, a smart watch, a smart phone, a tablet, and/or a computer system controlling an external display) separate from the computer system; and a representation of a fourth set of user body temperature information (e.g., a fourth period of time during which body temperature information was collected from the user (e.g., a third period of time different from the second period of time)) different from the second set of user body temperature information and collected by a third external device (e.g., a wearable device, a smart watch, a smart phone, a tablet, and/or a computer system controlling an external display) different from the second external device and the computer system (e.g., data chart 614d in FIG. 6L and/or data chart 614d in FIG. 6N displays data aggregated from multiple external devices (e.g., multiple wearable devices, such as device 800)). In some embodiments, the body temperature user interface includes and/or aggregates user body temperature information collected by a plurality of external devices (e.g., a plurality of external devices associated with the computer system and/or associated with a particular user of the computer system). Allowing a user to collect body temperature information using multiple external devices improves the user-device interface and helps the user to provide correct inputs.

Note that details of the processes described above with respect to method 700 (e.g., FIG. 7) are also applicable in an analogous manner to the methods described below. For example, method 900, method 950, and/or method 1100 optionally include one or more of the characteristics of the various methods described above with reference to method 700. For example, the body temperature information that is collected in method 700 can be used to generate health-based notifications and/or predictions in methods 900, 950, and/or 1100. For brevity, these details are not repeated below.

FIGS. 8A-8K illustrate exemplary user interfaces for generating and providing ovulation date predictions, in accordance with some embodiments. In some embodiments, ovulation date predictions are generated based on user body temperature information. The user interfaces in these figures are used to illustrate the processes described below, including the processes in FIGS. 9A and 9B.

FIG. 8A illustrates electronic device 600 with touch-sensitive display 602. FIGS. 8A-8C illustrate example user interfaces that allow a user to provide information (e.g., health information and/or biometric information) pertaining to and/or relevant to menstrual cycle predictions, including ovulation date predictions and fertile window predictions. In FIG. 8A, electronic device 600 displays health factors user interface 807, which asks the user to specify, via options 808a-808d, whether one or more predetermined health factors are applicable to the user. Option 808a is selectable by a user to indicate that the user is pregnant. Option 808b is selectable by a user to indicate that the user is lactating. Option 808c is selectable by a user to indicate that the user is using contraception. Option 808d is selectable by a user to indicate that none of these health factors currently apply to the user and, in FIG. 8A, has been selected by the user. Health factors user interface 807 includes option 808e that is selectable to move to a next user interface (e.g., a next user interface in a series of onboarding user interfaces and/or a next user interface in a series of settings user interfaces), option 808f that is selectable to skip entering predetermined health factor information, option 808g that is selectable to return to a previous user interface, and option 808h that is selectable to cease display of health factors user interface 807. In some embodiments, electronic device 600 does not generate one or more menstrual cycle predictions (e.g., menstrual period predictions, ovulation date predictions, and/or fertile window predictions) if the user indicates that one or more predetermined health factors are applicable to the user during the relevant prediction time period. At FIG. 8A, electronic device 600 detects user input 810 (e.g., a tap input and/or a touch input) corresponding to selection of option 808e.

At FIG. 8B, in response to user input 810, electronic device 600 displays user interface 812. User interface 812 includes option 814a and option 814b. Option 814a is selectable by a user to selectively enable or disable menstrual period predictions. When option 814a is enabled (e.g., when menstrual period predictions are enabled), electronic device 600 predicts when the user's next menstrual period will occur (e.g., based on biometric information (e.g., body temperature information) and/or based on the user's previous menstrual cycle history). When option 814a is disabled (e.g., when menstrual period predictions are disabled), electronic device 600 does not generate and/or display menstrual period predictions. Option 814b is selectable by a user to selectively enable or disable menstrual period notifications. When option 814b is enabled (e.g., when menstrual period notifications are enabled), electronic device 600 generates and/or displays notifications (e.g., push notifications, lock screen notifications, and/or banner notifications) that are indicative of a menstrual period prediction (e.g., a prediction of when the user's next menstrual period will occur). When option 814b is disabled (e.g., when menstrual period notifications are disabled), electronic device 600 does not generate and/or display notifications (e.g., push notifications, lock screen notifications, and/or banner notifications) pertaining to menstrual period predictions and/or menstrual period data. User interface 812 also includes option 814c that is selectable to display a next user interface (e.g., a next user interface in a series of onboarding user interfaces and/or a next user interface in a series of settings user interfaces), option 814d that is selectable to display a previous user interface (e.g., user interface 807), and option 814e that is selectable to cease display of user interface 812. At FIG. 8B, electronic device 600 detects user input 816 (e.g., a tap input and/or a touch input) corresponding to selection of option 814c.

At FIG. 8C, in response to user input 816, electronic device 600 displays user interface 818. User interface 818 includes options 820a-820g. Option 820a is selectable by a user to selectively enable or disable fertile window predictions. When option 820a is enabled (e.g., when fertile window predictions are enabled), electronic device 600 generates and displays fertile window predictions (e.g., based on biometric information (e.g., body temperature information) and/or based on the user's menstrual cycle history). In various embodiments, fertile window predictions include one or more of: a prediction of when the user's next fertile window will occur, a prediction of when the user's previous fertile window occurred, and/or a prediction of when the user's current fertile window began. When option 820a is disabled (e.g., when fertile window predictions are disabled), electronic device 600 does not generate and/or display fertile window predictions. Option 820b is selectable by a user to selectively enable or disable fertile window notifications. When option 820b is enabled (e.g., when fertile window notifications are enabled), electronic device 600 generates and/or displays notifications (e.g., push notifications, lock screen notifications, and/or banner notifications) that are indicative of a fertile window prediction. When option 820b is disabled, electronic device 600 does not generate and/or display notifications pertaining to fertile window predictions and/or fertile window data. Option 820c is selectable by a user to selectively enable or disable fertility logging. When option 820c is enabled (e.g., fertility logging is enabled), the user is provided with options (e.g., electronic device 600 displays options) that allow the user to log ovulation tests and other fertility indicators, and displays indications of fertility indicators logged by a user (e.g., within cycle tracking user interface 828, described below). When option 820c is disabled (e.g., fertility logging is disabled), electronic device 600 does not display and/or provide the user with the option to log ovulation tests and/or other fertility indicators. Option 820d is selectable by a user to selectively enable or disable sexual activity logging. When option 820d is enabled (e.g., sexual activity logging is enabled), electronic device 600 displays and/or provides the user with options that allow the user to log sexual activity information (e.g., allows the user to identify dates on which the user had sex). When option 820d is disabled (e.g., sexual activity logging is disabled), electronic device 600 does not display and/or provide the user with options that allow the user to log sexual activity information. Option 820e is selectable to return to a previous user interface (e.g., user interface 812), option 820f is selectable to cease display of user interface 818, and option 820g is selectable to display a next user interface (e.g., a next user interface in a series of onboarding user interfaces and/or a next user interface in a series of settings user interfaces).

User interface 818 also includes notification 822, which indicates that the user can improve period predictions, ovulation predictions, and fertile window predictions by setting up sleep tracking, and wearing their wearable device (e.g., watch) to sleep at night (e.g., so that electronic device 600 can log and track body temperature information, which can be used to improve these predictions). In some embodiments, notification 822 is displayed in response to a determination that the user has not enabled sleep tracking, has not enabled body temperature logging, and/or has not consistently logged body temperature information. Notification 822 includes option 824b, that is selectable to display sleep tracking user interface 618 (discussed above), and option 824a, that is selectable to cease display of notification 822. At FIG. 8C, electronic device 600 detects user input 826 (e.g., a tap input and/or a touch input) corresponding to selection of option 820g.

At FIG. 8D, in response to user input 826, electronic device 600 displays cycle tracking user interface 828. Cycle tracking user interface 828 includes first region 830, which includes day representations 832a-832g. Each day representation 832a-832g is representative of a single calendar day (e.g., representation 832d is representative of Friday May 6, representation 832c is representative of Thursday May 5, representation 832e is representative of Saturday May 7, and so forth). A user is able to navigate within first region 830 (e.g., by scrolling and/or swiping to the left or right) to view additional day representations representative of dates in the past and/or in the future in order to view and/or log menstrual cycle information about a particular date. Indication 831a is pointing to representation 832d, indicating that representations 832d is the currently selected day representation, and indication 831b indicates that representation 832d is representative of “Today, May 6.” Accordingly, with representation 832d being currently selected, a user is able to interact with options 834a-834d to log menstrual cycle information for Friday, May 6. If the user swipes to the right to select representation 832c (e.g., to move representation 832c below indication 831a), the user is able to interact with options 834a-834d to log menstrual cycle information for Thursday, May 5. Option 834a is selectable to initiate a process for indicating that the user experienced menstruation on the currently selected date. Option 834b is selectable to initiate a process for logging one or more symptoms for the selected date. Option 834c is selectable to initiate a process for indicating that the user experienced spotting on the currently selected date. Option 834d is selectable to initiate a process for indicating that one or more predetermined health factors (e.g., pregnancy, using contraception, lactation, etc.) were applicable to the user on the selected date.

Option 834e is selectable to view one or more health-related predictions for the user, including, for example, menstrual period predictions (e.g., when the user will have her next menstrual period), fertile window predictions (e.g., when the user will have her next fertile window and/or when the user's most recent fertile window occurred), and ovulation date predictions (e.g., a prediction of when the user last ovulated and/or when the user will ovulate next). Such predictions are also shown via daily representations 832a-832g in region 830. In FIG. 8D, representations 832a-832e are displayed in a first manner (e.g., in a first color, in a first pattern, etc.) indicating that electronic device 600 has predicted that the user's fertile window has taken place and/or will take place on those dates (e.g., in FIG. 8D, Tuesday May 3 to Saturday May 7).

Cycle tracking user interface 828 also includes option 834g that is selectable to return to a previously-displayed user interface, and option 834f that is selectable to initiate a process for logging a menstrual cycle (e.g., for identifying one or more dates on which the user menstruated).

Cycle tracking user interface 828 also includes history section 836, which displays menstrual cycle information for one or more menstrual cycles. History section 836 includes first region 838a, which corresponds to a current menstrual cycle (e.g., which started on April 17 and has continued to the present date, May 6), and second region 838b, which corresponds to a previous menstrual cycle that occurred from March 19 to April 16. First region 838a includes 20 pill-shaped day representations, with each day representation representative of a single day in the current menstrual cycle (e.g., the first pill representative of the first day, the second pill representative of the second day, and so forth). In FIG. 8D, the first six day representations 835a in region 838a indicate that the user menstruated for those first six days (e.g., by displaying the small circle at the top of each day representation), and the final six day representations 835b in region 838a indicate that electronic device 600 predicts that the user's fertile window has occurred during those six days (e.g., based on the user's past menstrual cycle information). Similarly, in region 838b, there are 28 pill-shaped day representations to represent the 28 days in that menstrual cycle (not all of the day representations are displayed in FIG. 8D, and, in some embodiments, the user is able to swipe in region 838b to reveal the additional day representations). Region 838b indicates that, during the previous menstrual cycle, the user menstruated in the first five days (e.g., based on the small circle shown at the top of those day representations), the user exhibited spotting on the third and fifth days (e.g., based on the small dot at the bottom of the third and fifth day representations), and show a fertile window prediction for days 10-16 of the menstrual cycle (based on the single diagonal hatching shown in those day representations), and shows an ovulation date prediction for the 15^{th} day of the menstrual cycle (e.g., based on the double diagonal hatching shown on that day representation). In some embodiments, when fertility predictions are not enabled, cycle tracking user interface 828 does not display fertile window predictions or ovulation date predictions. In some embodiments, when fertility predictions are not enabled, cycle tracking user interface 828 displays menstrual period predictions. In some embodiments, when fertility predictions are enabled, cycle tracking user interface 828 does not display menstrual period predictions, and only displays fertile window predictions and ovulation date predictions.

As discussed above, in FIG. 8D, electronic device 600 has generated a fertile window prediction for the user's current menstrual cycle (e.g., day representations 832a-832e in region 830, and day representations 835b in region 838a). In the depicted example scenario, the fertile window prediction shown in FIG. 8D represents an initial fertile window prediction that is generated with information pertaining to the user (e.g., based on menstrual cycle history information for the user), but is not generated using user body temperature information. In the example shown in FIG. 8D, the user has enabled sleep tracking and has enabled body temperature collection and logging, as was discussed above with reference to FIGS. 6A-6Q. In FIGS. 8E-8G, an example scenario is demonstrated in which user body temperature information is used by electronic device 600 to generate an ovulation date prediction, and to update the initial fertile window prediction based on the ovulation date prediction. In some embodiments, when sleep tracking and/or body temperature logging are not enabled, cycle tracking user interface 828 includes a notification similar to notification 822 in FIG. 8C informing the user that the user can improve menstrual cycle predictions (e.g., menstrual period predictions, ovulation date predictions, and/or fertile window predictions) by setting up sleep tracking, setting up body temperature logging, and/or wearing their wearable device to sleep each night.

At FIG. 8E, electronic device 600 displays wrist temperature user interface 612, which was discussed above with reference to FIGS. 6A-6Q. In FIG. 8E, wrist temperature user interface 612 indicates that the user's relative body temperature spiked around May 3. In FIG. 8E, based on the user's body temperature information, electronic device 600 generates an ovulation date prediction predicting that the user ovulated on May 3.

In FIG. 8F, electronic device 600 displays lock screen user interface 840, which includes indication 842a indicating the electronic device 600 is in a locked state. Lock screen user interface also includes current time indication 842b, current date indication 842c, flashlight option 842d, and camera option 842e. In FIG. 8F, after electronic device 600 generates the ovulation date prediction of May 3, electronic device 600 displays push notification 842f while electronic device 600 is in the locked state. In some embodiments, push notification 842f causes display 602 to transition from an inactive state (e.g., an off state) to an active state (e.g., an on state). Push notification 842f informs the user that electronic device 600 has determined (e.g., based on the user's body temperature information), that it is likely that the user ovulated on May 3.

FIG. 8F also depicts electronic device 800, which is a smart watch with touch-sensitive display 802, rotatable and depressible input mechanism 804, and button 806. In some embodiments, electronic device 800 corresponds to electronic device 600 (e.g., is paired to electronic device 600 and/or corresponds to the same user as electronic device 600). In response to and/or after electronic device 600 determines that it is likely that the user ovulated on May 3, electronic device 800 displays notification 844 informing the user of the ovulation date prediction. At FIG. 8F, electronic device 600 detects user input 846 (e.g., a touch input and/or a tap input) corresponding to selection of notification 842f.

At FIG. 8G, in response to user input 846, electronic device 600 displays cycle tracking user interface 828. In FIG. 8G, cycle tracking user interface 828 has been updated based on the ovulation date prediction by electronic device 600. In FIG. 8D, prior to the ovulation date prediction, cycle tracking user interface 828 displayed an initial fertile window prediction, predicting the user's fertile window to take place between May 1 and May 7 (e.g., day representations 832a-832e, and day representations 835b). However, in FIG. 8G, after electronic device 600 has used the user's body temperature information to generate a prediction that the user ovulated on May 3, cycle tracking user interface 828 now displays an updated fertile window prediction 835c between April 28 and May 4. In some embodiments, the updated fertile window prediction is defined by selecting a predetermined number of days before the ovulation date prediction (e.g., the five days before the ovulation date prediction) and a predetermined number of days after the ovulation date prediction (e.g., the day after the ovulation date prediction). Accordingly, day representations 832j, 832i, 832h, and 832b are displayed in the manner indicative of a fertile window prediction (e.g., single line hatching) while day representations 832c and 832d, which were previously shown with the single line hatching representing fertile window prediction, are no longer filled with this pattern. Cycle tracking user interface 828 also displays the ovulation date prediction by displaying day representation 832a with double line hatching, and day representation 835d with double line hatching. In this way, from FIG. 8D to FIG. 8G, electronic device 600 went from displaying an initial fertile window prediction, to generating an ovulation date prediction using user body temperature information, to then displaying the ovulation date prediction and an updated fertile window prediction.

In FIG. 8H, electronic device 600 displays cycle history user interface 848, which includes menstrual cycle representations 850a-850d corresponding to a plurality of previous menstrual cycles for a user. In FIG. 8H, menstrual cycle representations 850a, 850c, and 850d do not include fertile window predictions or ovulation date predictions because the user indicated that one or more predetermined health factors (e.g., lactation and/or contraception user) were applicable to the user during those menstrual cycles. However, menstrual cycle representation 850b does include a fertile window prediction and an ovulation date prediction. At FIG. 8H, electronic device 600 detects user input 852 (e.g., a touch input and/or a tap input) corresponding to selection of menstrual cycle representation 850b.

At FIG. 8I, in response to user input 852, electronic device 600 displays cycle detail user interface 854. Cycle detail user interface 854 displays information pertaining to the menstrual cycle that was selected in cycle history user interface 848. In FIG. 8I, cycle detail user interface 854 displays details pertaining to a menstrual cycle that occurred from Dec. 14, 2021 to Jan. 15, 2022. Cycle history user interface 848 includes cycle length information 856a, a set of day representations 856b representative of each day in the menstrual cycle. Day representations 856b include indications of which days the user menstruated (e.g., days 1-6 in FIG. 8I), on which days the user logged spotting (e.g., days 3, 5, and 16 in FIG. 8I), an ovulation date prediction for the menstrual cycle (e.g., day 14 in FIG. 8I), and a fertile window prediction for the menstrual cycle (e.g., days 9-15 in FIG. 8I). Cycle detail user interface 854 also displays body temperature information 856c, which indicates the user's body temperature (e.g., relative body temperature) for each day in the menstrual cycle. Cycle detail user interface 854 also includes sexual history information 856d. Cycle detail user interface 854 also includes option 856e, which is selectable to initiate a process for exporting the menstrual cycle information in cycle detail user interface 854 (e.g., in a PDF file, in an email, or in another file format), option 856f that is selectable to initiate a process for editing the menstrual cycle information in cycle detail user interface 854, option 856g that is selectable to return to cycle history user interface 848.

FIG. 8J illustrates an alternative embodiment of cycle tracking user interface 828, in which cycle tracking user interface 828 includes option 858 that is selectable to log a positive ovulation prediction kit (OPK) test. FIG. 8J depicts the same scenario that was described above with reference to FIG. 8D. In FIG. 8J, electronic device 600 has generated an initial fertile window prediction, and displays the initial fertile window prediction for May 1-May 7. In FIG. 8J, while Friday May 6 is selected (e.g., as indicated in region 830 and by indications 831a, 831b), electronic device 600 detects user input 860 corresponding to selection of option 858.

At FIG. 8K, in response to user input 860, electronic device 600 logs a positive OPK test for Friday May 6, as indicated by indication 862. Furthermore, in response to user input 860, electronic device 600 marks Saturday May 7 (day representation 832e) as the predicted ovulation date (a positive OPK test is indicative of the day before ovulation), and updates the fertile window based on the new predicted ovulation date by marking May 2 through May 8 as the new predicted fertile window (e.g., within region 830). In some embodiments, a positive OPK test overrides any fertile window prediction or ovulation date prediction generated by electronic device 600. In some embodiments, even after electronic device 600 predicts an ovulation date for the user based on user body temperature information, a positive OPK test will override the ovulation date prediction made by electronic device 600.

FIG. 9A is a flow diagram illustrating a method for generating and providing ovulation date predictions using a computer system in accordance with some embodiments. Method 900 is performed at a computer system (e.g., 100, 300, 500) (e.g., 600, 800) (e.g., a wearable device, a smart watch, a smart phone, a tablet, and/or a computer system controlling an external display) that is in communication with a display generation component (e.g., 602, 802) (e.g., a display controller, a touch-sensitive display system; and/or a display (e.g., integrated and/or connected)) and one or more input devices (e.g., 601, 602, 802, 804, 806) (e.g., a touch-sensitive surface (e.g., a touch-sensitive display); an accelerometer; a rotatable input mechanism; a depressible input mechanism; and/or a rotatable and depressible input mechanism). Some operations in method 900 are, optionally, combined, the orders of some operations are, optionally, changed, and some operations are, optionally, omitted.

As described below, method 900 provides an intuitive way for generating and providing ovulation date predictions. The method reduces the cognitive burden on a user for generating and/or receiving ovulation date predictions, thereby creating a more efficient human-machine interface. For battery-operated computing devices, enabling a user to generate and/or receive ovulation date predictions faster and more efficiently conserves power and increases the time between battery charges.

The computer system (e.g., 600, 800) receives (902) (e.g., via the one or more input devices and/or via an external device (e.g., an external (e.g., separate) wearable device, smart watch, smart phone, tablet, and/or computer system controlling an external display) body temperature information corresponding to a user (e.g., body temperature measurements collected from the user over one or more periods of time). After (in some embodiments, in response to) receiving the body temperature information corresponding to the user (904), the computer system displays (906), via the display generation component (e.g., 602, 802), a push notification (e.g., 842f and/or 844) that causes the display generation component to transition from an inactive state (e.g., FIG. 10I) (e.g., off state and/or a state in which the display generation component does not display content) to an active state (e.g., FIG. 8F) (e.g., an on state and/or a state in which the display generation component displays content) and that indicates to the user that a predicted ovulation date has been determined for the user (e.g., has been determined by the computer system and/or by an external device), wherein the predicted ovulation date was determined based on the body temperature information corresponding to the user. In some embodiments, after (in some embodiments, in response to) receiving the body temperature information corresponding to the user, the computer system determines, based on the body temperature information corresponding to the user, a predicted ovulation date for the user (e.g., a predicted ovulation date for the user that is determined based on changes in the body temperature of the user over a period of time). In some embodiments, the computer system displays the push notification that indicates to the user that the computer system has determined a predicted ovulation date after (in some embodiments, in response to) determining the predicted ovulation date for the user. Automatically displaying a push notification that indicates to the user that a predicted ovulation date has been determined for the user after receiving body temperature information corresponding to the user informs the user of an ovulation date prediction with fewer user inputs, thereby reducing the number of user inputs required to perform an operation. Displaying the push notification that indicates to the user that a predicted ovulation date has been determined for the user provides the user with visual feedback about the state of the system (e.g., that the system has determined a predicted ovulation date for the user), thereby providing improved visual feedback to the user.

In some embodiments, displaying the push notification comprises displaying the push notification while maintaining the computer system in a locked state (e.g., lock screen user interface 840 and/or indication 842a indicate that computer system 600 is in a locked state in FIG. 8F) (e.g., a state in which one or more features and/or one or more sets of content of the computer system are inaccessible to a user and/or a state in which authentication information is required to transition the computer system to an unlocked state). Automatically displaying a push notification that indicates to the user that a predicted ovulation date has been determined for the user after receiving body temperature information corresponding to the user informs the user of an ovulation date prediction with fewer user inputs, thereby reducing the number of user inputs required to perform an operation.

In some embodiments, receiving the body temperature information corresponding to the user (e.g., user body temperature information shown in data chart 614d) comprises receiving the body temperature information corresponding to the user from a first external device (e.g., 800) (e.g., a wearable device, a smart watch, a smart phone, a tablet, and/or a computer system controlling an external display) separate from (e.g., different from) the computer system. Automatically displaying a push notification that indicates to the user that a predicted ovulation date has been determined for the user after receiving body temperature information corresponding to the user from an external device informs the user of an ovulation date prediction with fewer user inputs, thereby reducing the number of user inputs required to perform an operation.

In some embodiments, the computer system is in communication with a first sensor (e.g., a first temperature sensor and/or a first body temperature sensor) (e.g., a first sensor built into the computer system (e.g., contained within a housing corresponding to the computer system), connected to the computer system, and/or separate from the computer system) (e.g., a first sensor built into electronic device 600 and/or built into electronic device 800); and receiving the body temperature information corresponding to the user comprises receiving the body temperature information corresponding to the user from the first sensor. Automatically displaying a push notification that indicates to the user that a predicted ovulation date has been determined for the user after receiving body temperature information corresponding to the user from a first sensor informs the user of an ovulation date prediction with fewer user inputs, thereby reducing the number of user inputs required to perform an operation.

In some embodiments, while displaying the push notification (e.g., 842f and/or 844) (908): the computer system receives (910), via the one or more input devices, a selection input (e.g., 846) (e.g., one or more touch inputs, one or more tap inputs, one or more mouse inputs, one or more keyboard inputs, and/or one or more gesture inputs) corresponding to selection of the push notification. In response to receiving the selection input corresponding to selection of the push notification (912), the computer system displays (914), via the display generation component, a cycle tracking user interface (e.g., 828) that includes (e.g., displays and/or shows) the predicted ovulation date (e.g., day representation 832a in FIG. 8G and/or day representation 835d in FIG. 8G). In some embodiments, the push notification does not display the predicted ovulation date. In some embodiments, the cycle tracking user interface that includes the predicted ovulation date is displayed in response to receiving the selection input corresponding to selection of the push notification and in accordance with a determination that device security criteria are satisfied (e.g., in accordance with a determination that authentication information (e.g., passcode information and/or biometric authentication information (e.g., face scan authentication, iris scan authentication, and/or fingerprint scan authentication)) satisfies authentication criteria). Displaying a push notification that is selectable to display the predicted ovulation date allows a user to view the predicted ovulation date with fewer user inputs, thereby reducing the number of user inputs required to perform an operation.

In some embodiments, the push notification (e.g., 842f and/or 844) includes (e.g., displays and/or shows) the predicted ovulation date (e.g., “May 3” in FIG. 8F). Displaying a push notification that displays the predicted ovulation date allows a user to view the predicted ovulation date with fewer user inputs, thereby reducing the number of user inputs required to perform an operation. Displaying a push notification that displays the predicted ovulation date provides the user with visual feedback about the state of the system (e.g., that the system has determined a predicted ovulation date for the user), thereby providing improved visual feedback to the user.

In some embodiments, displaying the push notification (e.g., 842f and/or 844) is performed in accordance with a determination that the user has enabled a fertility tracking setting (e.g., 820a, 820b, and/or 820c) (e.g., has enabled a setting that indicates that the user authorizes receipt of fertility information and/or authorizes performance of ovulation date predictions). In some embodiments, after receiving the body temperature information corresponding to the user, and in accordance with a determination that the user has not enabled the fertility tracking setting, the computer system forgoes displaying the push notification. Automatically displaying a push notification that indicates to the user that a predicted ovulation date has been determined for the user after receiving body temperature information corresponding to the user and in accordance with a determination that the user has enabled fertility tracking informs the user that an ovulation date prediction is ready for the user to view with fewer user inputs, thereby reducing the number of user inputs required to perform an operation.

In some embodiments, displaying the push notification (e.g., 842f and/or 844) is performed in accordance with a determination that a threshold amount of body temperature information corresponding to the user has been collected (e.g., in FIGS. 6L-6N, the threshold amount of body temperature information has been collected, whereas in FIGS. 6K and 6Q, the threshold amount of body temperature information has not been collected) (e.g., body temperature information corresponding to the user has been collected for a threshold number of days and/or a body temperature information corresponding to the user has been collected a threshold number of times). In some embodiments, after receiving the body temperature information corresponding to the user, and in accordance with a determination that the threshold amount of body temperature information corresponding to the user has not been collected, the computer system forgoes displaying the push notification. Automatically displaying a push notification that indicates to the user that a predicted ovulation date has been determined for the user after receiving body temperature information corresponding to the user and in accordance with a determination that a threshold amount of body temperature information corresponding to the user has been collected informs the user that an ovulation date prediction is ready for the user to view with fewer user inputs, thereby reducing the number of user inputs required to perform an operation. Displaying the push notification only if a threshold amount of body temperature information has been collected improves the user-device interface by avoiding presenting the user with information that does not apply to the user and helping the user to provide correct inputs.

In some embodiments, displaying the push notification (e.g., 842f and/or 844) is performed in accordance with a determination that one or more predetermined health factors do not apply to the user (e.g., FIG. 8A and/or “factors” option 834d in FIG. 8D) (e.g., the user is not pregnant, the user is not using contraception, and/or the user is not using a first type of contraception). In some embodiments, after receiving the body temperature information corresponding to the user, and in accordance with a determination that at least one of the one or more predetermined health factors apply to the user (e.g., the user is pregnant and/or the user is using contraception), the computer system forgoes displaying the push notification. Automatically displaying a push notification that indicates to the user that a predicted ovulation date has been determined for the user after receiving body temperature information corresponding to the user and in accordance with a determination that the one or more predetermined health factors do not apply to the user informs the user that an ovulation date prediction is ready for the user to view with fewer user inputs, thereby reducing the number of user inputs required to perform an operation. Displaying the push notification only if one or more predetermined health factors do not apply to the user improves the user-device interface by avoiding presenting the user with information that does not apply to the user and helping the user to provide correct inputs.

In some embodiments, the computer system receives, via the one or more input devices, an input (e.g., a first set of user inputs and/or one or more user inputs) (e.g., one or more touch inputs, one or more non-touch inputs, and/or one or more gestures) corresponding to a request to enter a sleep tracking mode (e.g., 638a and/or 638b and/or one or more user inputs defining a sleep schedule (e.g., sleep schedule shown in 636i, 636j)) (e.g., a mode and/or state in which one or more notifications (e.g., notifications of a first type) received by the computer system are suppressed and/or in which user sleep information (e.g., sleep quality information) is collected). In response to the input, the computer system enters the sleep tracking mode (e.g., sleep focus state in FIGS. 6H-6I), wherein receiving the body temperature information corresponding to the user is performed while the computer system is in the sleep tracking mode. In some embodiments, receiving the body temperature information corresponding to the user is performed in accordance with a determination that the computer system is in the sleep tracking mode. Automatically collecting body temperature information in response to the computer system entering the sleep tracking mode allows for collection of body temperature information with fewer user inputs, thereby reducing the number of user inputs required to perform an operation.

In some embodiments, the computer system receives notification data of a first type (e.g., notifications of a type that are subject to being suppressed while in the sleep tracking mode) (in some embodiments, all notifications are suppressed in the sleep tracking mode) (in some embodiments, notifications of a second type (e.g., emergency notifications, wake alarm notifications), are not suppressed while in the sleep tracking mode). In response to receiving the notification data of a first type: in accordance with a determination that the computer system is not in the sleep tracking mode (e.g., option 636a is in the inactive and/or off state) (e.g., when the notification data of the first type is received), the computer system outputs a first notification (e.g., displays a first notification; causes the display generation component to transition from an inactive state (e.g., off and/or a state in which content is not displayed on the display generation component) to an active state (e.g., on and/or a state in which content is displayed); outputs an audio output; and/or outputs a haptic output) corresponding to the notification data of the first type; and in accordance with a determination that the computer system is in the sleep tracking mode (e.g., electronic device 600 in FIGS. 6H-6I) (e.g., option 636a is in the active and/or on state) (e.g., when the notification data of the first type is received), the computer system forgoes outputting (e.g., suppresses) the first notification corresponding to the notification data of the first type (e.g., forgoes displaying the first notification; forgoes causing the display generation component to transition from an inactive state to an active state; forgoes outputting an audio output; and/or forgoes outputting a haptic output). Automatically suppressing notifications when the computer system is in the sleep tracking mode allows a user to perform this operation with fewer user inputs, thereby reducing the number of user inputs required to perform an operation.

In some embodiments, in response to receiving the notification data of the first type: in accordance with a determination that the computer system is not in the sleep tracking mode (e.g., option 636a is in the inactive and/or off state) (e.g., when the notification data of the first type is received), the computer system causes a second external device (e.g., 800) (e.g., a wearable device, a smart watch, a smart phone, a tablet, and/or a computer system controlling an external display) separate from (e.g., different from) the computer system to display a second notification corresponding to the notification data of the first type (e.g., causes external device 800 to display and/or output a notification); and in accordance with a determination that the computer system is in the sleep tracking mode (e.g., option 636a is in the active and/or on state) (e.g., when the notification data of the first type is received), the computer system forgoes causing the second external device to display the second notification corresponding to the notification data of the first type (e.g., in FIG. 6H, electronic device 800 is also in the sleep focus state when electronic device 600 is in the sleep focus state (e.g., based on option 630e being enabled)). Automatically suppressing notifications on the second external device when the computer system is in the sleep tracking mode allows a user to perform this operation with fewer user inputs, thereby reducing the number of user inputs required to perform an operation.

In some embodiments, receiving the body temperature information corresponding to the user (e.g., body temperature information shown in data chart 614d) comprises receiving the body temperature information corresponding to the user from a third external device (e.g., 800) (e.g., a wearable device, a smart watch, a smart phone, a tablet, and/or a computer system controlling an external display) separate from (e.g., different from) the computer system. Automatically displaying a push notification that indicates to the user that a predicted ovulation date has been determined for the user after receiving body temperature information corresponding to the user from an external device informs the user of the predicted ovulation date with fewer user inputs, thereby reducing the number of user inputs required to perform an operation.

In some embodiments, the computer system receives notification data of a second type (e.g., notifications of a type that are subject to being suppressed while in the sleep tracking mode) (in some embodiments, all notifications are suppressed in the sleep tracking mode) (in some embodiments, notifications of a third type (e.g., emergency notifications, wake alarm notifications), are not suppressed while in the sleep tracking mode). In response to receiving the notification data of a second type: in accordance with a determination that the computer system (e.g., 600) is not in the sleep tracking mode (e.g., option 636a is in the inactive and/or off state) (e.g., when the notification data of the second type is received), the computer system (e.g., 600) causes the third external device (e.g., 800) to output a third notification (e.g., causes the third external device to display a third notification; causes a display generation component corresponding to and/or in communication with the third external device to transition from an inactive state (e.g., off and/or a state in which content is not displayed on the display generation component) to an active state (e.g., on and/or a state in which content is displayed); causes the third external device to output an audio output; and/or causes the third external device to output a haptic output) corresponding to the notification data of the third type; and in accordance with a determination that the computer system is in the sleep tracking mode (e.g., option 636a is in the active and/or on state) (e.g., when the notification data of the second type is received), the computer system (e.g., 600) forgoes causing the third external device (e.g., 800) to output the third notification corresponding to the notification data of the second type (e.g., when notifications are suppressed on electronic device 600, notifications are also suppressed on electronic device 800 (e.g., based on setting 630e being enabled)). Automatically suppressing notifications on the third external device when the computer system is in the sleep tracking mode allows a user to perform this operation with fewer user inputs, thereby reducing the number of user inputs required to perform an operation.

In some embodiments, the computer system (e.g., 600) receives (e.g., via the one or more input devices and/or via an external device (e.g., an external (e.g., separate) wearable device, smart watch, smart phone, tablet, and/or computer system controlling an external display)) second body temperature information corresponding to the user (e.g., body temperature measurements collected from the user over one or more periods of time) (e.g., second body temperature information different from the body temperature information). After (in some embodiments, in response to) receiving the second body temperature information corresponding to the user: in accordance with a determination that the user has not logged (e.g., entered and/or provided) an ovulation prediction kit test result during a predefined period of time (e.g., no OPK test result is logged in FIG. 8D) (e.g., within a threshold amount of time (e.g., in the last two weeks, in the last three weeks, in the last four weeks)), the computer system displays, via the display generation component (e.g., 602), a second push notification (e.g., 842f and/or 844) that causes the display generation component to transition from the inactive state to the active state and that indicates to the user that a second predicted ovulation date has been determined for the user, wherein the second predicted ovulation date was determined based on the second body temperature information corresponding to the user; and in accordance with a determination that the user has logged an ovulation prediction kit test result during the predefined period of time (e.g., user logs positive OPK test in FIGS. 8J-8K), the computer system forgoes displaying the second push notification (e.g., a push notification indicative of a predicted ovulation date is not generated after the user logs the positive OPK test in FIGS. 8J-8K). Automatically displaying a second push notification that indicates to the user that a second predicted ovulation date has been determined for the user after receiving second body temperature information corresponding to the user informs the user that an ovulation date prediction is ready for them to view with fewer user inputs, thereby reducing the number of user inputs required to perform an operation. Forgoing displaying the second push notification if the user has logged an ovulation prediction kit test result during the predefined period of time improves the user-device interface by avoiding presenting the user with information that does not apply to the user and helping the user to provide correct inputs.

Note that details of the processes described above with respect to method 900 (e.g., FIG. 9A) are also applicable in an analogous manner to the methods described above and/or below. For example, method 700, method 950, and/or method 1100 optionally include one or more of the characteristics of the various methods described above with reference to method 900. For example, the body temperature information that is collected in method 700 can be used to generate health-based notifications and/or predictions in methods 900, 950, and/or 1100. For brevity, these details are not repeated below.

FIG. 9B is a flow diagram illustrating a method for generating and providing ovulation date predictions using a computer system in accordance with some embodiments. Method 950 is performed at a computer system (e.g., 100, 300, 500) (e.g., 600, 800) (e.g., a wearable device, a smart watch, a smart phone, a tablet, and/or a computer system controlling an external display) that is in communication with a display generation component (e.g., 602, 802) (e.g., a display controller, a touch-sensitive display system; and/or a display (e.g., integrated and/or connected)) and one or more input devices (e.g., 601, 602, 802, 804, 806) (e.g., a touch-sensitive surface (e.g., a touch-sensitive display); an accelerometer; a rotatable input mechanism; a depressible input mechanism; and/or a rotatable and depressible input mechanism). Some operations in method 950 are, optionally, combined, the orders of some operations are, optionally, changed, and some operations are, optionally, omitted.

As described below, method 950 provides an intuitive way for generating and providing ovulation date predictions. The method reduces the cognitive burden on a user for generating and/or receiving ovulation date predictions, thereby creating a more efficient human-machine interface. For battery-operated computing devices, enabling a user to generate and/or receive ovulation date predictions faster and more efficiently conserves power and increases the time between battery charges.

The computer system (e.g., 600) displays (952), via the display generation component (e.g., 602), at a first time, a cycle tracking user interface (e.g., 828) (e.g., a cycle tracking user interface corresponding to a user) including displaying a first fertile window prediction for a user (e.g., 835b and/or day representations 832a-832e in FIG. 8D) (e.g., a prediction of one or more days and/or dates during which the user has an increased likelihood of becoming pregnant (e.g., a prediction of the date on which the user will release an egg from an ovary and a predetermined period of time prior to that date (e.g., five dates prior to that date)) (e.g., a fertile window prediction corresponding to a first period of time). The computer system receives (954) (e.g., via the one or more input devices and/or via an external device (e.g., an external (e.g., separate) wearable device, smart watch, smart phone, tablet, and/or computer system controlling an external display)) body temperature information corresponding to the user (e.g., body temperature information shown in data chart 614d in FIG. 8E) (e.g., body temperature measurements collected from the user over one or more periods of time). After (in some embodiments, in response to) receiving the body temperature information corresponding to the user (956), the computer system determines (958), at a second time subsequent to the first time, based on the body temperature information corresponding to the user, a predicted ovulation date for the user (e.g., notification 842f and/or 844) (e.g., a predicted ovulation date for the user that is determined based on changes in the body temperature of the user over a period of time). The computer system displays (960), via the display generation component, at a third time subsequent to the second time, an updated cycle tracking user interface (e.g., 828 in FIG. 8G) (e.g., an updated and/or modified version of the cycle tracking user interface (e.g., updated and/or modified based on the body temperature information and/or based on the predicted ovulation date for the user)), including concurrently displaying: a representation of the predicted ovulation date for the user (e.g., 832a, 835d) (e.g., a graphical object indicative of the predicted ovulation date for the user); and an updated fertile window prediction for the user (e.g., day representations 832j, 832i, 832h, 832b, and/or 835c) different from the first fertile window prediction for the user (e.g., an updated, revised, and/or modified fertile window prediction (e.g., updated, revised, and/or modified based on the body temperature information and/or the predicted ovulation date for the user)). In some embodiments, the first fertile window prediction is determined based on a set of information that does not include the body temperature information corresponding to the user and/or does not include any body temperature information corresponding to the user (e.g., the first fertile window prediction is determined based on historical menstrual cycle information corresponding to the user). In some embodiments, the predicted ovulation date for the user is determined, based on the body temperature information corresponding to the user, by an external device and transmitted to and received by the computer system. Automatically updating the cycle tracking user interface with an updated fertile window prediction after receiving body temperature information corresponding to the user allows the user to view updated fertile window prediction information with fewer user inputs, thereby reducing the number of user inputs required to perform an operation. Automatically updating the cycle tracking user interface with an updated fertile window prediction after receiving body temperature information corresponding to the user provides the user with visual feedback about the state of the system (e.g., that the system has determined a predicted ovulation date for the user and/or an updated fertile window prediction for the user), thereby providing improved visual feedback to the user.

In some embodiments, receiving the body temperature information corresponding to the user comprises receiving the body temperature information corresponding to the user from a first external device (e.g., 800) (e.g., a wearable device, a smart watch, a smart phone, a tablet, and/or a computer system controlling an external display) separate from (e.g., different from) the computer system. Automatically updating the cycle tracking user interface with an updated fertile window prediction after receiving body temperature information corresponding to the user allows the user to view updated fertile window prediction information with fewer user inputs, thereby reducing the number of user inputs required to perform an operation.

In some embodiments, the computer system is in communication with a first sensor (e.g., a sensor built into electronic device 600 and/or electronic device 800) (e.g., a first temperature sensor and/or a first body temperature sensor) (e.g., a first sensor built into the computer system (e.g., contained within a housing corresponding to the computer system), connected to the computer system, and/or separate from the computer system); and receiving the body temperature information corresponding to the user comprises receiving the body temperature information corresponding to the user from the first sensor. Automatically updating the cycle tracking user interface with an updated fertile window prediction after receiving body temperature information corresponding to the user allows the user to view updated fertile window prediction information with fewer user inputs, thereby reducing the number of user inputs required to perform an operation.

In some embodiments, the representation of the predicted ovulation date (e.g., 832a and/or 835d) for the user is displayed with a first visual appearance (e.g., with a first set of visual characteristics (e.g., color, size, shape, font, and/or pattern)); and the updated fertile window prediction for the user (e.g., 832j, 832i, 832h, 832b, 832c, and/or 835c) is displayed with a second visual appearance (e.g., with a second set of visual characteristics (e.g., color, size, shape, font, and/or pattern)) different from the first visual appearance. Displaying the representation of the predicted ovulation date with a first visual appearance and displaying the updated fertile window prediction with a second visual appearance different from the first visual appearance allows a user to visually distinguish between the predicted ovulation date and the fertile window prediction without additional user inputs and also provides improved visual feedback.

In some embodiments, the representation of the predicted ovulation date for the user (e.g., 832a and/or 835d) includes a first color (e.g., at least a portion of the representation of the predicted ovulation date for the user is displayed in the first color); and the updated fertile window prediction for the user (e.g., 832j, 832i, 832h, 832b, 832c, and/or 835c) includes a second color different from the first color (e.g., at least a portion of the updated fertile window prediction is displayed in the second color) (in some embodiments, the updated fertile window prediction does not include the first color). Displaying the representation of the predicted ovulation date in a first color and displaying the updated fertile window prediction in a second color different from the first color allows a user to visually distinguish between the predicted ovulation date and the fertile window prediction without additional user inputs.

In some embodiments, the computer system (e.g., 600) displays, via the display generation component (e.g., 602), at a fourth time subsequent to the third time, the cycle tracking user interface (e.g., 828), including: in accordance with a determination that a fertility tracking setting (e.g., 820a, 820b, and/or 820c) is enabled (e.g., a setting that indicates that the user authorizes receipt of fertility information and/or authorizes performance of ovulation date predictions) (e.g., at the fourth time), displaying (e.g., within the cycle tracking user interface) a second fertile window prediction (e.g., day representations 832a, 832b, 832c, 832d, 832e, 835b and/or 835c) for the user (e.g., a second fertile window prediction that is different from or the same as the first fertile window prediction and/or the updated fertile window prediction); and in accordance with a determination that the fertility tracking setting is not enabled (e.g., at the fourth time), displaying (e.g., within the cycle tracking user interface) a menstrual period prediction (e.g., 835a) for the user (e.g., a prediction of one or more days during which the user has an increased likelihood of menstruating) without displaying the second fertile window prediction for the user (in some embodiments, without displaying any fertile window prediction for the user). Displaying a fertile window prediction for a user when a fertility tracking setting is enabled, and displaying a menstrual period prediction for the user when the fertility tracking setting is not enabled, improves the user-device interface by avoiding presentation of information that is not relevant and/or of interest to the user.

In some embodiments, the computer system receives second body temperature information corresponding to the user (e.g., body temperature information shown in data chart 614d) (e.g., second body temperature information different from or the same as the first body temperature information). After receiving the second body temperature information corresponding to the user, the computer system displays, via the display generation component, the cycle tracking user interface (e.g., 828), including: in accordance with a determination that a fertility tracking setting (e.g., 820a, 820b, and/or 820c) is enabled (e.g., a setting that indicates that the user authorizes receipt of fertility information and/or authorizes performance of ovulation date predictions), displaying (e.g., within the cycle tracking user interface) a representation of a second predicted ovulation date (e.g., 832a, 835d) for the user, wherein the second predicted ovulation date is determined based on the second body temperature information; and in accordance with a determination that the fertility tracking setting is not enabled, forgoing displaying the representation of the second predicted ovulation date for the user. Displaying a predicted ovulation date for a user when a fertility tracking setting is enabled, and forgoing displaying the predicted ovulation date when the fertility tracking setting is not enabled, improves the user-device interface by avoiding presentation of information that is not relevant and/or of interest to the user.

In some embodiments, the computer system receives third body temperature information corresponding to the user (e.g., body temperature information shown in data chart 614d) (e.g., third body temperature information different from or the same as the first body temperature information and/or the second body temperature information). After receiving the third body temperature information corresponding to the user: in accordance with a determination that one or more predetermined health factors (e.g., FIG. 8A and/or “factors” option 834d) do not apply to the user (e.g., the user is not pregnant, the user is not using contraception, and/or the user is not using a first type of contraception) (e.g., at the time of receiving the third body temperature information and/or at a defined time subsequent to receiving the third body temperature information corresponding to the user), the computer system determines, based on the third body temperature information, a third predicted ovulation date (e.g., 842f, 844, 832a, and/or 835d) for the user (and, optionally, displays a representation of and/or an indication of the third predicted ovulation date); and in accordance with a determination that at least one of the one or more predetermined health factors apply to the user (e.g., the user is pregnant and/or the user is using contraception) (e.g., at the time of receiving the third body temperature information and/or at a defined time subsequent to receiving the third body temperature information corresponding to the user), the computer system forgoes determining a predicted ovulation date for the user based on the third body temperature information. Automatically determining a predicted ovulation date for a user in accordance with a determination that one or more predetermined health factors do not apply to the user reduces the number of user inputs required to perform an operation. Furthermore, forgoing performing an ovulation date prediction for a user if one or more predetermined health factors apply to the user improves the user-device interface by avoiding presentation of information that does not apply to the user.

In some embodiments, the computer system receives fourth body temperature information corresponding to the user (e.g., body temperature information shown in data chart 614d) (e.g., fourth body temperature information different from or the same as the first body temperature information, the second body temperature information, and/or the third body temperature information). After receiving the fourth body temperature information corresponding to the user: in accordance with a determination that a threshold amount of body temperature information (e.g., user body temperature information associated with a user of the computer system, collected from the user of the computer system and/or collected by the computer system) has been collected (e.g., greater than a threshold number of days of body temperature information has been collected and/or greater than a threshold number of hours of body temperature information has been collected), the computer system determines, based on the fourth body temperature information, a fourth predicted ovulation date for the user (e.g., 842f, 844, 832a, and/or 835d) (and, optionally, displays a representation of and/or an indication of the fourth predicted ovulation date); and in accordance with a determination that the threshold amount of body temperature information has not been collected, the computer system forgoes determining a prediction ovulation date for the user based on the fourth body temperature information. Automatically determining a predicted ovulation date for a user in accordance with a determination that a threshold amount of body temperature information has been collected reduces the number of user inputs required to perform an operation. Furthermore, forgoing performing an ovulation date prediction for a user if the threshold amount of body temperature information has not been collected improves the user-device interface by avoiding presentation of information that does not apply to the user and/or that is inaccurate.

In some embodiments, the computer system receives, via the one or more input devices, an input (e.g., a first set of user inputs and/or one or more user inputs) (e.g., one or more touch inputs, one or more non-touch inputs, and/or one or more gestures) corresponding to a request to enter a sleep tracking mode (e.g., 638a and/or 638b and/or one or more user inputs defining a sleep schedule (e.g., sleep schedule shown in 636i, 636j)) (e.g., a mode and/or state in which one or more notifications (e.g., notifications of a first type) received by the computer system are suppressed and/or in which user sleep information (e.g., sleep quality information) is collected). In response to the input, the computer system enters the sleep tracking mode (e.g., sleep focus state in FIGS. 6H-6I), wherein: receiving the body temperature information corresponding to the user is performed while the computer system is in the sleep tracking mode (in some embodiments, receiving the body temperature information corresponding to the user is performed in accordance with a determination that the computer system is in the sleep tracking mode). Automatically collecting body temperature information in response to the computer system entering the sleep tracking mode allows for collection of body temperature information with fewer user inputs, thereby reducing the number of user inputs required to perform an operation.

In some embodiments, the computer system receives notification data of a first type (e.g., notifications of a type that are subject to being suppressed while in the sleep tracking mode) (in some embodiments, all notifications are suppressed in the sleep tracking mode) (in some embodiments, notifications of a second type (e.g., emergency notifications, wake alarm notifications), are not suppressed while in the sleep tracking mode). In response to receiving the notification data of a first type: in accordance with a determination that the computer system is not in the sleep tracking mode (e.g., option 636a is in the inactive and/or off state) (e.g., when the notification data of the first type is received), the computer system outputs a first notification (e.g., displays a first notification; causes the display generation component to transition from an inactive state (e.g., off and/or a state in which content is not displayed on the display generation component) to an active state (e.g., on and/or a state in which content is displayed); outputs an audio output; and/or outputs a haptic output) corresponding to the notification data of the first type; and in accordance with a determination that the computer system is in the sleep tracking mode (e.g., option 636a is in the active and/or on state) (e.g., when the notification data of the first type is received), the computer system forgoes outputting (e.g., suppresses) the first notification corresponding to the notification data of the first type (e.g., forgoes displaying the first notification; forgoes causing the display generation component to transition from an inactive state to an active state; forgoes outputting an audio output; and/or forgoes outputting a haptic output). Automatically suppressing notifications when the computer system is in the sleep tracking mode allows a user to perform this operation with fewer user inputs, thereby reducing the number of user inputs required to perform an operation.

In some embodiments, in response to one or more user inputs (e.g., one or more touch inputs, one or more non-touch inputs, and/or one or more gesture inputs) on a second external device (e.g., 800) (e.g., a wearable device, a smart watch, a smart phone, a tablet, and/or a computer system controlling an external display) separate from the computer system corresponding to a request to enter a sleep tracking mode (e.g., a mode and/or state in which one or more notifications (e.g., notifications of a first type) received by the computer system are suppressed and/or in which user sleep information (e.g., sleep quality information) is collected), the computer system enters the sleep tracking mode (e.g., a user is able to provide one or more user inputs on electronic device 800 to cause electronic device 600 to enter the sleep focus state), wherein: receiving the body temperature information corresponding to the user comprises receiving the body temperature information corresponding to the user from the second external device; and the second external device collects the body temperature information corresponding to the user while the computer system is in the sleep tracking mode. In some embodiments, the second external device collects the body temperature information corresponding to the user in accordance with a determination that the computer system is in the sleep tracking mode. Automatically collecting body temperature information from an external device in response to the computer system entering the sleep tracking mode allows for collection of body temperature information with fewer user inputs, thereby reducing the number of user inputs required to perform an operation.

In some embodiments, the computer system receives notification data of a second type (e.g., notifications of a type that are subject to being suppressed while in the sleep tracking mode) (in some embodiments, all notifications are suppressed in the sleep tracking mode) (in some embodiments, notifications of a third type (e.g., emergency notifications, wake alarm notifications), are not suppressed while in the sleep tracking mode). In response to receiving the notification data of a second type: in accordance with a determination that the computer system is not in the sleep tracking mode (e.g., when the notification data of the second type is received), the computer system (e.g., 600) causes the second external device (e.g., 800) to output a second notification (e.g., causes the second external device to display a second notification; causes a display generation component corresponding to and/or in communication with the second external device to transition from an inactive state (e.g., off and/or a state in which content is not displayed on the display generation component) to an active state (e.g., on and/or a state in which content is displayed); causes the second external device to output an audio output; and/or causes the second external device to output a haptic output) corresponding to the notification data of the second type; and in accordance with a determination that the computer system (e.g., 600) is in the sleep tracking mode (e.g., when the notification data of the second type is received), the computer system (e.g., 600) forgoes causing the second external device (e.g., 800) to output the second notification corresponding to the notification data of the second type. Automatically suppressing notifications on the second external device when the computer system is in the sleep tracking mode allows a user to perform this operation with fewer user inputs, thereby reducing the number of user inputs required to perform an operation.

In some embodiments, after displaying the updated cycle tracking user interface (e.g., 828 in FIG. 8G), the computer system receives (e.g., via one or more user inputs, from an application, and/or from an external device) OPK (e.g., ovulation prediction kit) test result information corresponding to the user (e.g., user input 860 in FIG. 8J indicating a positive OPK test on Friday, May 6). After (in some embodiments, in response to) receiving the OPK test result information corresponding to the user, the computer system determines, based on the OPK test result information, an updated predicted ovulation date for the user (e.g., in FIG. 8K, electronic device 600 has determined that the predicted ovulation date for the user is Saturday, May 7 based on the user input 860). After determining the updated predicted ovulation date for the user, the computer system displays, via the display generation component, a second updated cycle tracking user interface (e.g., 828 in FIG. 8K) (e.g., an updated, revised, and/or modified version of the cycle tracking user interface; and/or an updated, revised, and/or modified version of the updated cycle tracking user interface (e.g., updated and/or modified based on the ovulation prediction kit test result information)), including: a representation of the updated predicted ovulation date for the user (e.g., 832e in FIG. 8K) (in some embodiments, electronic device 600 goes from displaying cycle tracking user interface 828 in FIG. 8G, with a first predicted ovulation date (e.g., 832a in FIG. 8G) to displaying cycle tracking user interface 828 in FIG. 8K that displays a different and/or updated predicted ovulation date (e.g., 832e in FIG. 8K)) (e.g., a graphical object indicative of the updated predicted ovulation date) (optionally, without displaying the representation of the predicted ovulation date for the user). In some embodiments, displaying the second updated cycle tracking user interface further includes displaying (e.g., concurrently with the representation of the updated predicted ovulation date for the user) a second updated fertile window prediction for the user, wherein the second updated fertile window prediction is determined based on the updated predicted ovulation date for the user. Automatically determining an updated predicted ovulation date for a user based on an OPK test result allows a user to receive updated ovulation date predictions with fewer user inputs, thereby reducing the number of user inputs required to perform an operation.

In some embodiments, displaying the second updated cycle tracking user interface (e.g., 828 in FIG. 8K) further comprises: displaying, concurrently with the representation of the updated predicted ovulation date for the user (e.g., 832e), a second updated fertile window prediction for the user (e.g., 832a, 832b, 832c, 832d, 832f and/or fertile window prediction in region 838a in FIG. 8K) (e.g., a second updated fertile window prediction different from the updated fertile window prediction and/or the same as the updated fertile window prediction), wherein the second updated fertile window prediction is determined based on the OPK test result information. In some embodiments, the second updated fertile window prediction is determined based on the updated predicted ovulation date for the user. Automatically updated a fertile window prediction based on an OPK test result allows a user to receive updated fertile window predictions with fewer user inputs, thereby reducing the number of user inputs required to perform an operation.

In some embodiments, the representation of the predicted ovulation date for the user (e.g., 832a in FIG. 8G) is displayed with a first set of visual characteristics (e.g., color, size, and/or shape); and the representation of the updated predicted ovulation date for the user (e.g., 832e in FIG. 8K) is displayed with the first set of visual characteristics (e.g., the same color, size, and/or shape). Automatically determining an updated predicted ovulation date for a user based on an OPK test result allows a user to receive updated ovulation date predictions with fewer user inputs, thereby reducing the number of user inputs required to perform an operation.

Note that details of the processes described above with respect to method 950 (e.g., FIG. 9B) are also applicable in an analogous manner to the methods described above and/or below. For example, method 700, method 900, and/or method 1100 optionally include one or more of the characteristics of the various methods described above with reference to method 950. For example, the body temperature information that is collected in method 700 can be used to generate health-based notifications and/or predictions in methods 900, 950, and/or 1100. For brevity, these details are not repeated below.

FIGS. 10A-10N illustrate exemplary user interfaces for generating and providing health-related notifications, in accordance with some embodiments. The user interfaces in these figures are used to illustrate the processes described below, including the processes in FIG. 11.

FIG. 10A illustrates electronic device 600 with touch-sensitive display 602. At FIG. 10A, electronic device 600 displays cycle tracking user interface 828, which was discussed above with reference to FIGS. 8A-8K. In FIG. 10A, the current date is June 6, and in cycle tracking user interface 828, day representation 1001a representing the current date is selected (e.g., as indicated by indications 831a, 831b).

FIG. 10A also illustrates electronic device 800, which is a smart watch with touch-sensitive display 802, rotatable and depressible input mechanism 804, and button 806. In the depicted embodiments, electronic device 800 corresponds to electronic device 600 (e.g., electronic device 800 is paired to electronic device 600 and/or corresponds to the same user as electronic device 600). FIGS. 10A-10E illustrate electronic device 600 and electronic device 800 displaying substantially identical user interfaces to demonstrate that, in certain embodiments, a user is able to log and/or enter menstruation information using either electronic device 600 or electronic device 800.

In FIG. 10A, electronic device 800 displays cycle tracking user interface 1000, which is substantially identical in substance to cycle tracking user interface 828. Cycle tracking user interface 1000 includes region 1002 with a plurality of day representations (similar to region 830 in cycle tracking user interface 828), option 1004a (which corresponds to option 832a), option 1004b (which corresponds to option 834b), and option 1004c (which corresponds to option 834c). In FIG. 10A, day representation 1001b, representative of the current day, is selected within cycle tracking user interface 1000. In FIG. 10A, while day representation 1001a is selected, electronic device 600 detects user input 1005 (e.g., a tap input and/or a touch input) corresponding to selection of option 834a, indicative of a user request to log menstruation information for the selected day. Furthermore, while day representation 1001b is selected, electronic device 800 detects user input 1006 (e.g., a tap input and/or a touch input) corresponding to selection of option 1004a.

At FIG. 10B, in response to user input 1005, electronic device 600 displays user interface 1008, and in response to user input 1006, electronic device 800 displays user interface 1012. User interface 1012 corresponds to user interface 1008 (e.g., user interface 1012 is a watch version of user interface 1008 and/or smaller form factor version of user interface 1008). User interface 1008 includes option 1010a that is selectable by a user to indicate that the user is experiencing light flow, option 1010b that is selectable by a user to indicate that the user is experiencing medium flow, option 1010c that is selectable by a user to indicate that the user is experiencing heavy flow, option 1010d that is selectable by a user to indicate that the user is experiencing unspecified flow, and option 1010e that is selectable by a user to indicate that the user is experiencing no flow. User interface 1008 also includes option 1010f that is selectable to cease display of user interface 1008 (and, for example, cancel entry of menstruation information and return to cycle tracking user interface 828), and option 1010g that is selectable to advance to a next user interface. User interface 1012 includes options 1014a-1014g that correspond to options 1010a-1010g, respectively. In FIG. 10B, on both electronic device 600 and electronic device 800, the user has specified that the user is experiencing light flow. Electronic device 600 detects user input 1016a (e.g., a touch input and/or a tap input) corresponding to selection of option 1010g, and electronic device 800 detects user input 1016b (e.g., a touch input and/or a tap input) corresponding to selection of option 1014g.

At FIG. 10C, in response to user input 1016a, electronic device 600 displays user interface 1018, and in response to user input 1016b, electronic device 800 displays user interface 1022. User interface 1022 corresponds to user interface 1018 (e.g., user interface 1022 is a watch version of user interface 1018 and/or smaller form factor version of user interface 1018). User interface 1018 includes various options 1020a-1020e, and user interface 1022 includes corresponding options 1024a-1024g, that are selectable by a user to indicate that the user is experience one or more symptoms (e.g., cramps, mood changes, low back pain, aches, bloating, constipation, headache). In FIG. 10C, on both electronic device 600 and electronic device 800, the user selects five symptoms (cramps, mood changes, aches, bloating, and heading (the final two symptoms are not visible on electronic device 600 as depicted in FIG. 10C)). After the user has selected their symptoms, electronic device 600 detects user input 1026a (e.g., a touch input and/or a tap input) corresponding to selection of option 1020f, and electronic device 800 detects user input 1026b (e.g., a touch input and/or a tap input) corresponding to selection of option 1024h.

At FIG. 10D, in response to user input 1026a, electronic device 600 displays user interface 1028, and in response to user input 1026b, electronic device 800 displays user interface 1032. User interface 1032 corresponds to user interface 1028 (e.g., user interface 1032 is a watch version of user interface 1028 and/or smaller form factor version of user interface 1028). User interface 1028 includes option 1030a that is selectable by a user to indicate that the user is experiencing spotting, and user interface 1032 includes corresponding option 1034a. In FIG. 10D, the user is not experiencing spotting, and has not selected option 1030a or option 1034a. At FIG. 10D, electronic device 600 detects user input 1036a (e.g., a touch input and/or a tap input) corresponding to option 1030c, and electronic device 800 detects user input 1036b (e.g., a touch input and/or a tap input) corresponding to option 1034c.

At FIG. 10E, in response to user input 1036a, electronic device 600 logs the menstruation information entered by the user, and displays cycle tracking user interface 828. In FIG. 10E, cycle tracking user interface 828 has been updated according to the menstruation information entered by the user in FIGS. 10B-10D. For example, day representation 1001a is now shown with a menstruation indication (e.g., hashed circle at the top of day representation 1001a) indicating that the user has logged menstruation for that day, option 834a is shown with “light flow”, and option 834b is shown indicating that the user has logged 5 symptoms for that day. Similarly, in response to user input 1036b, electronic device 800 logs the menstruation information entered by the user, and displays cycle tracking user interface 1000, which has also been updated according to the menstruation information entered by the user in the same ways described above with reference to cycle tracking user interface 828.

FIG. 10E depicts an example scenario in which the user has entered menstruation information, and electronic device 600 (and/or electronic device 800) have not detected any potential health issues based on the menstruation information entered by the user. However, FIG. 10F depicts an alternative scenario in which electronic device 600 detects a potential health issue based on the menstruation information that has just been entered by the user.

At FIG. 10F, in response to user input 1036a, electronic device 600 displays cycle tracking user interface 828, which is nearly identical to cycle tracking user interface 828 as it was described in FIG. 10E. However, in FIG. 10F, electronic device 600 has identified, based on the menstrual cycle information entered by the user in FIGS. 10B-10D, a potential health issue for the user. Accordingly, in FIG. 10F, electronic device 600 displays, within cycle tracking user interface 828, notification 1038a indicating that the user may have a potential health issue. In some embodiments, notification 1038a does not identify the potential health issue, and the user is not presented with the potential health issue until the user confirms that the underlying menstrual cycle information that led to identification of the potential health issue is accurate. Notification 1038a includes option 1038c that is selectable to dismiss notification 1038a and/or cease display of notification 1038a, and option 1038b that is selectable to review the user's cycle history to confirm whether or not the user's cycle history is accurate.

In FIG. 10F, electronic device 800 does not display a notification of a potential health issue. In some embodiments, electronic device 600 displays notification 1038a indicative of a potential health issue, but a corresponding device (e.g., a corresponding wearable device) such as electronic device 800 does not display a notification of a potential health issue. In some embodiments, when a user ignores and/or does not interact with notification 1038a for a duration of time, and new menstrual cycle information entered by the user (e.g., either revising previously entered menstrual cycle information and/or entering new menstrual cycle information on future days) negates the potential health issue identified by electronic device 600, electronic device 600 ceases display of notification 1038a within cycle tracking user interface 828. At FIG. 10F, electronic device 600 detects user input 1040 (e.g., a touch input and/or a tap input) corresponding to selection of option 1038b.

At FIG. 10G, in response to user input 1040, electronic device 600 displays user interface 1042. User interface 1042 displays menstrual cycle information 1044a, which includes the underlying menstrual cycle information that resulted in electronic device 600 detecting a potential health issue. In various embodiments, menstrual cycle information 1044a shown in user interface 1042 varies based on the potential health issue identified by electronic device 600. For example, if the duration of the user's periods are determined to be problematic, then menstrual cycle information 1044a shows the period durations that resulted in identification of the potential health issue, whereas if the frequency of the user's spotting is determined to be problematic, then menstrual cycle information 1044a shows the dates on which the user logged spotting. User interface 1042 instructs the user to review menstrual cycle information 1044a, and either confirm that menstrual cycle information 1044a is accurate (e.g., by selecting option 1044b), or revise and/or modify menstrual cycle information 1044a (e.g., by selecting option 1044c). User interface 1042 also includes option 1044d that is selectable to cease display of user interface 1042 without confirming or modifying menstrual cycle information 1044a (and, for example, return to cycle tracking user interface 828). At FIG. 10G, electronic device 600 detects user input 1046 (e.g., a touch input and/or a tap input) corresponding to selection of option 1044d.

At FIG. 10H, electronic device 600 displays health summary user interface 604, which was discussed above with reference to FIGS. 6A-6Q. In some embodiments, when a potential health issue for the user is determined based on menstrual cycle information logged by the user, a notification of the potential health issue is also displayed in health summary user interface 604. Accordingly, in FIG. 10H, electronic device 600 displays notification 1048a in health summary user interface 604. Notification 1048a includes option 1048c that is selectable to cease display of notification 1048a, and option 1048b that is selectable to display user interface 1042. In FIG. 10H, the user does not take any action with respect to notification 1048a (e.g., the user ignores notification 1048a). In some embodiments, when a user ignores and/or does not interact with notification 1048a for a duration of time, and new menstrual cycle information entered by the user (e.g., either revising previously entered menstrual cycle information and/or entering new menstrual cycle information on future days) negates the potential health issue identified by electronic device 600, electronic device 600 ceases display of notification 1048c within health summary user interface 604.

At FIG. 10I, electronic device 600 is in an inactive and locked state, and does not display content on display 602. At FIG. 10J, electronic device 600 receives and/or generates information pertaining to a push notification indicating a potential health issue for the user. At FIG. 10J, in response to receiving and/or generating this information, electronic device 600 displays lock screen user interface 840, which includes push notification 1052 informing the user that electronic device 600 has detected a potential health issue based on menstrual cycle information entered by the user. In some embodiments, push notification 1052 causes display 602 to transition from an inactive state (e.g., FIG. 10I) to an active state (e.g., FIG. 10J). In some embodiments, push notification 1052 is generated in accordance with a determination that the user has not yet confirmed or otherwise negated the potential health issue identified for the user (e.g., in accordance with a determination that there is still an unconfirmed and/or uncleared potential health issue identified for the user). In some embodiments, after electronic device 600 generates and/or displays push notification 1052, electronic device 600 does not display and/or the user is not presented with another push notification for the same unconfirmed potential health issue for a threshold duration of time (e.g., for one month and/or for sixty days). At FIG. 10J, electronic device 600 detects user input 1054 (e.g., a touch input and/or a tap input) corresponding to selection of push notification 1052.

At FIG. 10K, in response to user input 1054, electronic device 600 displays user interface 1042, which was described above with reference to FIG. 10G. In FIG. 10K, electronic device 600 detects user input 1056 (e.g., a touch input and/or a tap input) corresponding to selection of option 1044b, indicating that the user confirms the accuracy of menstrual cycle information 1044a.

At FIG. 10L, in response to user input 1056, and based on a determination that the user has confirmed menstrual cycle information 1044a, electronic device 600 displays user interface 1058. User interface 1058 includes indication 1060a, which identifies the potential health issue detected by electronic device 600, as well as the criteria that were used to determine the potential health issue. In FIG. 10L, electronic device 600 has determined that, within the past 180 days, the user has had two periods that lasted longer than 10 days and, accordingly, the user may have prolonged menstrual periods that the user should discuss with a doctor. User interface 1058 includes instruction 1060b, which instructs the user to contact a doctor. User interface 1058 also includes option 1060c, that is selectable to initiate a process for exporting menstrual cycle information for the user (e.g., export a PDF file that contains all menstrual cycle information pertaining to the user over the previous 12 months that is available to electronic device 600). User interface 1058 also includes option 1060d that is selectable to cease display of user interface 1058, and option 1060e that is selectable to return to user interface 1042.

FIG. 10L shows the potential health issue of “prolonged periods,” and indication 1060a displays the criteria for this health issue. However, in other scenarios, electronic device 1058 is able to detect other health issues, and indication 1060a would display different information. For example, in one embodiment, the potential health issue is an irregular menstrual cycle, and the criteria for determining the irregular menstrual cycle includes a determination that the difference between the maximum menstrual cycle length and minimum menstrual cycle length for the user for a predetermined period of time is greater than a threshold value (e.g., the difference is greater than or equal to 17 days for the latest two non-overlapping 90 day windows). In another example, the potential health issue is an infrequent menstrual cycle, and the criteria for determining the infrequent menstrual cycle includes a determination that logged menstrual cycle information includes less than a threshold number of logged menstrual periods during a predetermined period of time (e.g., less than two logged menstrual periods in the latest two non-overlapping 90 day windows). In another example, the potential health issue is a frequent spotting, and the criteria for determining the frequent spotting includes a determination that logged menstrual cycle information includes a threshold number of spotting instances within a predefined period of time (e.g., one or more days of spotting logged non-adjacent to a menstrual period in the latest two non-overlapping 90 day windows).

In some embodiments, if the user selects option 1044c in FIG. 10K rather than option 1044b, and modifies the underlying menstrual cycle information in such a way that negates the determination of a potential health issue, electronic device 600 displays an indication that the potential health issue does not apply to the user (and does not display user interface 1058). However, if the user selects option 1044c, and modifies the underlying menstrual cycle information, and the modified menstrual cycle information still indicates a potential health issue for the user, electronic device 600 displays user interface 1058.

In some embodiments, when a user confirms a potential health issue (e.g., by confirming the underlying menstrual cycle information, as shown in FIGS. 10K-10L), the potential health issue is displayed in cycle history user interface 848 (of FIG. 8H) and/or cycle detail user interface 854 (of FIG. 8I). In some embodiments, unconfirmed potential health issues are not displayed in these user interfaces, but confirmed potential health issues are displayed in these user interfaces. Furthermore, in some embodiments, for confirmed potential health issues that occurred within a threshold period of time (e.g., within the past 6 months or the past 12 months), cycle history user interface 848 and/or cycle detail user interface 854 displays an option that is selectable to export a 12-month menstrual cycle history (similar to option 1060c). However, for confirmed potential health issues that did not occur within the threshold period of time (e.g., occurred more than 6 months ago or more than 12 months ago), cycle history user interface 848 and/or cycle detail user interface 854 does not display an option that is selectable to export the 12-month menstrual cycle history.

In some embodiments, notification 1038a and/or notification 1052 are not displayed and/or generated in response to a lack of data entry by a user and/or a lack of interaction by a user in order to avoid false positives and/or inaccurate notifications. In other words, in some embodiments, notifications of potential health issues are only generated based on menstrual cycle information that is entered by a user.

In some embodiments, notifications of potential health issues (e.g., notification 1038a and/or push notification 1052) are not generated and/or displayed if the user indicates that one or more predetermined health factors (e.g., use of contraception and/or pregnancy) are applicable to the user and/or have been applicable to the user within a threshold duration of time (e.g., within the last 12 weeks).

FIG. 10M depicts electronic device 1070 with touch-sensitive display 1072. In FIG. 10M, electronic device 1070 displays sharing user interface 1074. Electronic device 1070 corresponds to a different user from electronic device 600. In the example scenario depicted, electronic device 600 corresponds to a user named Jane Appleseed, and electronic device 1070 corresponds to a user named Sarah. Jane Appleseed has chosen to share menstrual cycle information with Sarah. Accordingly, in FIG. 10M, sharing user interface 1074 includes object 1076a which indicates that Sarah is able to view menstrual cycle information corresponding to user Jane Appleseed. Furthermore, sharing user interface 1074 includes objects 1076b and 1076c, which indicate that the user Sarah is sharing menstrual cycle information with user “Mom” and is also sharing menstrual cycle information with Jane Appleseed. Object 1076a indicates that there are one or more alerts corresponding to user Jane Appleseed (e.g., based on the determination that one or more potential health issues have been identified for user Jane Appleseed based on menstrual cycle information corresponding to user Jane Appleseed). At FIG. 10M, electronic device 1070 detects user input 1078 (e.g., a touch input and/or a tap input) corresponding to selection of object 1076a.

At FIG. 10N, in response to user input 1078, electronic device 1070 displays user interface 1080. User interface 1080 corresponds to user Jane Appleseed, and includes indication 1082e which indicates that electronic device 600 has determined that Jane Appleseed has the potential health issue of prolonged periods. In some embodiments, indication 1082e is displayed in user interface 1080 only after the user of electronic device 600 confirms the potential health issue (e.g., by confirming the accuracy of the underlying menstrual cycle information). In some embodiments, unconfirmed potential health issues are not displayed in user interface 1080.

User interface 1080 also includes option 1082g that is selectable to cease display of indication 1082e, and option 1082f that is selectable to display additional details about the potential health issue identified for Jane Appleseed. User interface 1080 also includes options 1082a-1082d, 1082h, and 1082i. Option 1082a is selectable to initiate a process for sending a text message to user Jane Appleseed (e.g., display a text message user interface). Option 1082b is selectable to initiate a process for calling user Jane Appleseed (e.g., display a phone call user interface). Option 1082c is selectable to initiate a process for video calling user Jane Appleseed (e.g., display a video call user interface). Option 1082d is selectable to display contact information for user Jane Appleseed. Option 1082h displays ovulation prediction information for user Jane Appleseed. Option 1082i is selectable to return to user interface 1074.

FIG. 11 is a flow diagram illustrating a method for generating and providing health-related notifications using a computer system in accordance with some embodiments. Method 1100 is performed at a computer system (e.g., 100, 300, 500) (e.g., 600 and/or 800) (e.g., a wearable device, a smart watch, a smart phone, a tablet, and/or a computer system controlling an external display) that is in communication with a display generation component (e.g., 602 and/or 802) (e.g., a display controller, a touch-sensitive display system; and/or a display (e.g., integrated and/or connected)) and one or more input devices (e.g., 601, 602, 802, 804, and/or 806) (e.g., a touch-sensitive surface (e.g., a touch-sensitive display); an accelerometer; a rotatable input mechanism; a depressible input mechanism; and/or a rotatable and depressible input mechanism). Some operations in method 1100 are, optionally, combined, the orders of some operations are, optionally, changed, and some operations are, optionally, omitted.

As described below, method 1100 provides an intuitive way for generating and providing health-related notifications. The method reduces the cognitive burden on a user for generating and/or receiving health-related notifications, thereby creating a more efficient human-machine interface. For battery-operated computing devices, enabling a user to generate and/or receive health-related notifications faster and more efficiently conserves power and increases the time between battery charges.

The computer system (e.g., 600, 800) receives (1102), via the one or more input devices (e.g., 601, 602, 802, 804, and/or 806), menstrual cycle information (e.g., information identifying one or more dates during which a user was menstruating; and/or information identifying one or more dates during which a user exhibited spotting) for a user (e.g., inputted by the user via one or more user inputs) (e.g., one or more touch inputs, one or more non-touch inputs, and/or one or more gestures) (e.g., menstrual cycle information received in FIGS. 10A-10D). In accordance with a determination that the menstrual cycle information satisfies a first set of criteria (1104), the computer system displays (1106), via the display generation component, a push notification (e.g., 1052) (e.g., a notification that causes the display generation component to transition from an inactive (e.g., off state and/or a state in which the display generation component does not display content) to an active state (e.g., an on state and/or a state in which the display generation component displays content) in order to display the push notification and/or a notification that is generated by a second application and is overlaid on and/or displayed concurrently with a user interface corresponding to a first application that is different from the second application) that indicates to the user that the computer system has identified a potential health issue for the user. In some embodiments, in accordance with a determination that the menstrual cycle information does not satisfy the first set of criteria indicative of a potential health issue, the computer system forgoes displaying the push notification. Automatically displaying a push notification that indicates to the user that the computer system has identified a potential health issue for the user in accordance with a determination that the menstrual cycle information satisfies a first set of criteria informs the user of a potential health issue with fewer user inputs, thereby reducing the number of user inputs required to perform an operation. Automatically displaying a push notification that indicates to the user that the computer system has identified a potential health issue for the user in accordance with a determination that the menstrual cycle information satisfies a first set of criteria provides the user with visual feedback about the state of the system (e.g., that the system has identified a potential health issue for the user based on menstrual cycle information corresponding to the user), thereby providing improved visual feedback to the user.

In some embodiments, the push notification (e.g., 1052) causes the display generation component (e.g., 602) to transition from an inactive state (e.g., in FIG. 10I) (e.g., off state and/or a state in which the display generation component does not display content) to an active state (e.g., in FIG. 10J) (e.g., an on state and/or a state in which the display generation component displays content). Automatically displaying a push notification that transitions the display generation component from an inactive state to an active state and indicates to the user that the computer system has identified a potential health issue for the user in accordance with a determination that the menstrual cycle information satisfies a first set of criteria informs the user of a potential health issue with fewer user inputs, thereby reducing the number of user inputs required to perform an operation.

In some embodiments, displaying the push notification (e.g., 1052) comprises displaying the push notification while maintaining the computer system in a locked state (e.g., user interface 840 and/or indication 842a in FIG. 10J indicate that electronic device 600 is in a locked state) (e.g., a state in which one or more features and/or one or more sets of content of the computer system are inaccessible to a user and/or a state in which authentication information is required to transition the computer system to an unlocked state). Automatically displaying a push notification that indicates to the user that the computer system has identified a potential health issue for the user in accordance with a determination that the menstrual cycle information satisfies a first set of criteria, while maintaining the computer system in a locked state, informs the user of a potential health issue with fewer user inputs, thereby reducing the number of user inputs required to perform an operation.

In some embodiments, the first set of criteria includes a first criterion that is satisfied when a difference between a maximum menstrual cycle duration during a predetermined period of time (e.g., the duration of a menstrual cycle having the longest duration during the predetermined period of time (e.g., previous sixty days, previous ninety days, or previous 120 days)) and a minimum menstrual cycle duration during the predetermined period of time (e.g., the duration of a menstrual cycle having the shortest duration during the predetermined period of time) exceeds a threshold value (e.g., the difference is greater than or equal to 14 days, 15 days, 16 days, 17 days, 18 days, or 19 days). In some embodiments, the first set of criteria includes a second criterion that is satisfied when the first criterion is satisfied for multiple, consecutive pre-defined time periods (e.g., the first criterion is satisfied for the previous two non-overlapping 90 day windows) and the first set of criteria are satisfied when the first criterion and the second criterion are both satisfied (e.g., difference between maximum menstrual cycle duration and minimum menstrual cycle duration is greater than the threshold value for each of the last two non-overlapping 90 day windows). Displaying the push notification that indicates to the user that the computer system has identified a potential health issue for a user only when it is determined that the menstrual cycle information satisfies a first set of criteria improves the user-device interface by ensuring that a user is only presented with push notifications that are relevant to the user.

In some embodiments, the first set of criteria includes a second criterion that is satisfied when the menstrual cycle information for the user indicates that the user has experienced (e.g., the user has logged and/or entered) less than a threshold number of menstrual periods within a predetermined period of time (e.g., less than two logged menstrual periods in the latest two non-overlapping 90 day windows). Displaying the push notification that indicates to the user that the computer system has identified a potential health issue for a user only when it is determined that the menstrual cycle information satisfies a first set of criteria improves the user-device interface by ensuring that a user is only presented with push notifications that are relevant to the user.

In some embodiments, the first set of criteria includes a third criterion that is satisfied when the menstrual cycle information for the user indicates that, within a defined period of time (e.g., in the previous 180 days, in the previous 90 days, in the previous six months, in the previous three months, or in the previous 24 weeks), the user has experienced (e.g., the user has logged and/or entered) greater than a threshold number of menstrual periods that have had a duration that is greater than a threshold duration (e.g., two or more menstrual periods within the defined period of time that lasted 10 or more days, and/or two or more menstrual periods that started within the defined period of time (e.g., that started within the last 180 days) that lasted 10 or more days). Displaying the push notification that indicates to the user that the computer system has identified a potential health issue for a user only when it is determined that the menstrual cycle information satisfies a first set of criteria improves the user-device interface by ensuring that a user is only presented with push notifications that are relevant to the user.

In some embodiments, the first set of criteria includes a fourth criterion that is satisfied when the menstrual cycle information for the user indicates that, within a defined period of time (e.g., in the latest two non-overlapping 90 day windows and/or in the latest two non-overlapping three-month windows), the user has experienced (e.g., the user has logged and/or entered) greater than a threshold number of spotting instances that did not occur during a menstrual period and/or directly adjacent to (e.g., directly before and/or after; and/or the day before and/or after) a menstrual period (e.g., one or more days of spotting logged non-adjacent to a menstrual period in the latest two non-overlapping 90 day windows). Displaying the push notification that indicates to the user that the computer system has identified a potential health issue for a user only when it is determined that the menstrual cycle information satisfies a first set of criteria improves the user-device interface by ensuring that a user is only presented with push notifications that are relevant to the user.

In some embodiments, subsequent to displaying the push notification (e.g., 1052) (1108), the computer system receives (1110) second menstrual cycle information (e.g., additional menstrual cycle information, new menstrual cycle information, and/or second menstrual cycle information that includes the menstrual cycle information (and, in some embodiments, additional menstrual cycle information in addition to the menstrual cycle information)) for the user (e.g., user entering menstrual cycle information in FIGS. 10A-10D). Subsequent to receiving the second menstrual cycle information (1112) (in some embodiments, in response to receiving the second menstrual cycle information): in accordance with a determination that the second menstrual cycle information satisfies the first set of criteria and that a threshold duration of time has elapsed since the push notification (e.g., 1052) was displayed (1114) (e.g., greater than 30 days, greater than 60 days, or greater than 90 days), the computer system displays, via the display generation component, a second push notification (e.g., another push notification similar to and/or identical to push notification 1052) (e.g., a second push notification that causes the display generation component to transition from an inactive (e.g., off state and/or a state in which the display generation component does not display content) to an active state (e.g., an on state and/or a state in which the display generation component displays content) in order to display the second push notification and/or a second push notification that is generated by a second application and is overlaid on and/or displayed concurrently with a user interface corresponding to a first application that is different from the second application) that indicates to the user that the computer system has identified a potential health issue for the user; and in accordance with a determination that the second menstrual cycle information satisfies the first set of criteria and that less than the threshold duration of time has elapsed since the push notification was displayed (1116), the computer system forgoes displaying the second push notification. In some embodiments, subsequent to receiving the second menstrual cycle information, and in accordance with a determination that the second menstrual cycle information does not satisfy the first set of criteria, the computer system forgoes displaying the second push notification. Automatically forgoing display of the second push notification if less than a threshold duration of time has passed since the previous push notification improves the user-device interface by ensuring that a user is not inundated with too many push notifications pertaining to the same issue. Automatically displaying a second push notification that indicates to the user that the computer system has identified a potential health issue for the user in accordance with a determination that the second menstrual cycle information satisfies a first set of criteria informs the user of a potential health issue with fewer user inputs, thereby reducing the number of user inputs required to perform an operation.

In some embodiments, the computer system (e.g., 600 and/or 800) receives third menstrual cycle information (e.g., user entering menstrual cycle information in FIGS. 10A-10D) (e.g., additional menstrual cycle information, new menstrual cycle information, and/or third menstrual cycle information that includes the menstrual cycle information (and, in some embodiments, additional menstrual cycle information in addition to the menstrual cycle information)) for the user. Subsequent to receiving the third menstrual cycle information for the user: in accordance with a determination that the third menstrual cycle information was entered by a user (e.g., was inputted by a user and/or logged by a user), and that the third menstrual cycle information satisfies the first set of criteria, the computer system (e.g., 600) displays, via the display generation component (e.g., 602), a third push notification (e.g., 1052) (e.g., a notification that causes the display generation component to transition from an inactive (e.g., off state and/or a state in which the display generation component does not display content) to an active state (e.g., an on state and/or a state in which the display generation component displays content) in order to display the push notification and/or a notification that is generated by a second application and is overlaid on and/or displayed concurrently with a user interface corresponding to a first application that is different from the second application) that indicates to the user that the computer system has identified a potential health issue for the user; and in accordance with a determination that the third menstrual cycle information was not entered by a user (e.g., was not entered by a user and/or was not logged by the user), the computer system forgoes displaying the third push notification (e.g., regardless of whether or not the third menstrual cycle information satisfies the first set of criteria). In some embodiments, push notifications that indicate that the computer system has identified a potential health issue for the user are generated and/or displayed only based on user-inputted menstrual cycle information and/or user-confirmed menstrual cycle information, and are not generated and/or displayed based on a lack of interaction and/or a lack of data entry by a user (e.g., if the user has not logged and/or entered menstrual cycle information for a period of time, the computer system does not assume that the user has not menstruated and/or had a menstrual period for the period of time and does not generate push notifications based on the lack of data for the period of time). In some embodiments, the determination that the third menstrual cycle information was not entered by a user comprises a determination that no user input was received on any computer system to enter, log, and/or confirm the third menstrual cycle information. Displaying the push notification that indicates to the user that the computer system has identified a potential health issue for a user only when it is determined that the menstrual cycle information satisfies a first set of criteria improves the user-device interface by ensuring that a user is only presented with push notifications that are relevant to the user.

In some embodiments, the first set of criteria includes a sixth criterion that is satisfied when one or more predetermined health factors (e.g., health factors in FIG. 8A and/or “factors” option 834d) do not apply to the user (e.g., the user is not pregnant, the user is not using contraception, and/or the user is not using a first type of contraception). In some embodiments, when it is determined that at least one of the one or more predetermined health factors do apply to the user (e.g., the user is pregnant and/or the user is using contraception), the computer system forgoes displaying the push notification. Displaying the push notification only if one or more predetermined health factors do not apply to the user improves the user-device interface by avoiding presenting the user with information that does not apply to the user.

In some embodiments, the first set of criteria includes a seventh criterion that is satisfied when one or more predetermined health factors (e.g., health factors in FIG. 8A and/or “factors” option 834d) have not been applicable to the user for at least a threshold duration of time (e.g., the user has not been pregnant and/or has not used contraception for at least the threshold duration of time (e.g., 12 weeks, 3 months, and/or 90 days)). In some embodiments, when it is determined that at least one of the one or more predetermined health factors have been applicable to the user within the threshold duration of time (e.g., the user was pregnant and/or the user was using contraception within the previous 12 weeks, 3 months, and/or 90 days), the computer system forgoes displaying the push notification. Displaying the push notification only if one or more predetermined health factors have not been applicable to the user for at least the threshold duration of time improves the user-device interface by avoiding presenting the user with information that does not apply to the user.

In some embodiments, while displaying the push notification (e.g., 1052), the computer system (e.g., 600 and/or 800) receives, via the one or more input devices, a selection input (e.g., 1054) (e.g., one or more touch inputs, one or more non-touch inputs, and/or one or more gestures) corresponding to selection of the push notification. In response to receiving the selection input, the computer system displays, via the display generation component, a cycle history user interface (e.g., 1042), wherein the cycle history user interface includes: a first option (e.g., 1044b) that is selectable to confirm a cycle history of the user (e.g., a log and/or historical data containing historical menstrual cycle information for the user (e.g., for a predefined period of time (e.g., the last 3 months of cycle history and/or the last 6 months of cycle history)) (e.g., selectable to confirm that the cycle history of the user is accurate); and a second option (e.g., 1044c) that is selectable to revise the cycle history of the user (e.g., a second option that is selectable to display a history revision user interface that allows the user to correct and/or change menstrual cycle information for the user) (e.g., a second option that is selectable to indicate that the cycle history of the user is not accurate) (e.g., a second option that is selectable to initiate a process for revising the cycle history of the user). Automatically displaying the cycle history user interface that includes the first option and the second option in response to a selection input selecting the push notification allows a user to either confirm or revise the user's cycle history with fewer inputs, thereby reducing the number of inputs required to perform an operation.

In some embodiments, while displaying the cycle history user interface (e.g., 1042), the computer system receives, via the one or more input devices, a second selection input (e.g., 1056) (e.g., one or more touch inputs, one or more non-touch inputs, and/or one or more gestures) corresponding to selection of the first option (e.g., 1044b). In response to receiving the second selection input, the computer system displays, via the display generation component, first information identifying the potential health issue for the user (e.g., 1060a) (e.g., text and/or graphics identifying and/or naming the potential health issue for the user (e.g., irregular menstrual cycle, infrequent menstrual cycle, prolonged menstrual cycle, and/or frequent spotting)). Displaying the first information identifying the potential health issue for the user only after the user confirms the user's cycle history improves the user-device interface by ensuring that potentially inaccurate or faulty information is not presented to the user until the user confirms the underlying menstrual cycle information.

In some embodiments, in response to receiving the second selection input (e.g., 1056), the computer system displays (e.g., concurrently with the first information), via the display generation component, a third option (e.g., 1060c) that is selectable to initiate a process for exporting (e.g., printing, saving, sending, and/or transmitting) a file (e.g., a document file, a PDF file, and/or a graphics file) containing menstrual cycle history information corresponding to the user (e.g., a 12-month menstrual cycle history for the user). Automatically displaying the third option in response to the second selection input allows a user to export menstrual cycle history information with fewer inputs, thereby reducing the number of inputs required to perform an operation.

In some embodiments, while displaying the cycle history user interface (e.g., 1042), the computer system receives, via the one or more input devices, a third selection input (e.g., one or more touch inputs, one or more non-touch inputs, and/or one or more gestures) corresponding to selection of the second option (e.g., 1044c). Subsequent to receiving the third selection input, the computer system receives, via the one or more input devices, corrected menstrual cycle information (e.g., receiving one or more user inputs revising the cycle history of the user, correcting the cycle history of the user, and/or entering corrected menstrual cycle information) corresponding to the user. In response to receiving the corrected menstrual cycle information: in accordance with a determination that the corrected menstrual cycle information satisfies the first set of criteria, the computer system displays, via the display generation component, second information identifying the potential health issue for the user (e.g., 1060a) (e.g., text and/or graphics identifying and/or naming the potential health issue for the user (e.g., irregular menstrual cycle, infrequent menstrual cycle, prolonged menstrual cycle, and/or frequent spotting)); and in accordance with a determination that the corrected menstrual cycle information does not satisfy the first set of criteria, the computer system displays, via the display generation component, an indication that potential health issues have not been identified for the user (e.g., based on the corrected menstrual cycle information) (and, in some embodiments, forgoes displaying the second information identifying the potential health issue for the user). Displaying information identifying the potential health issue for the user only after the user confirms the user's cycle history (e.g., by providing corrected menstrual cycle information) improves the user-device interface by ensuring that potentially inaccurate or faulty information is not presented to the user until the user confirms the underlying menstrual cycle information.

In some embodiments, in response to receiving the corrected menstrual cycle information: in accordance with a determination that the corrected menstrual cycle information satisfies the first set of criteria, the computer system displays (e.g., concurrently with the second information), via the display generation component, an export option (e.g., 1060c) that is selectable to initiate a process for exporting (e.g., printing, saving, sending, and/or transmitting) a file (e.g., a document file, a PDF file, and/or a graphics file) containing menstrual cycle history information corresponding to the user (e.g., a 12-month menstrual cycle history for the user). Automatically displaying the export option in response to receiving the corrected menstrual cycle information and in accordance with a determination that the corrected menstrual cycle information satisfied the first set of criteria allows a user to export menstrual cycle history information with fewer inputs, thereby reducing the number of inputs required to perform an operation.

In some embodiments, subsequent to receiving the menstrual cycle information for the user (e.g., FIGS. 10A-10D), the computer system displays, via the display generation component, a cycle tracking user interface (e.g., 828) (e.g., a cycle tracking user interface corresponding to the user) (e.g., a cycle tracking user interface that includes one or more selectable objects that are selectable by a user to enter and/or log menstrual cycle information), wherein: in accordance with a determination that the menstrual cycle information satisfies the first set of criteria, displaying the cycle tracking user interface includes displaying (e.g., within the cycle tracking user interface) a first indicator (e.g., 1038a) (e.g., a visual object and/or visual indication) that indicates to the user that the computer system has identified a potential health issue for the user. In some embodiments, in accordance with a determination that the menstrual cycle information does not satisfy the first set of criteria, the cycle tracking user interface does not include the first indicator. Automatically displaying a first indicator that indicates to the user that the computer system has identified a potential health issue for the user in accordance with a determination that the menstrual cycle information satisfies a first set of criteria informs the user of a potential health issue with fewer user inputs, thereby reducing the number of user inputs required to perform an operation. Automatically displaying a first indicator that indicates to the user that the computer system has identified a potential health issue for the user in accordance with a determination that the menstrual cycle information satisfies a first set of criteria provides the user with visual feedback about the state of the system (e.g., that the system has identified a potential health issue for the user based on menstrual cycle information corresponding to the user), thereby providing improved visual feedback to the user.

In some embodiments, subsequent to displaying cycle tracking user interface (e.g., 828), including the first indicator (e.g., 1038a), the computer system receives, via the one or more input devices, second updated menstrual cycle information for the user (e.g., user entering updated menstrual cycle information, similar to FIGS. 10A-10D) (e.g., second updated menstrual cycle information that includes additional menstrual cycle information that is not included in the menstrual cycle information). Subsequent to receiving the second updated menstrual cycle information, the computer system re-displays the cycle tracking user interface (e.g., 828), wherein: in accordance with a determination that the second updated menstrual cycle information does not satisfy the first set of criteria, the cycle tracking user interface does not include the first indicator (e.g., 1038a) (e.g., cycle tracking user interface 828 in FIG. 10E does not include the first indicator 1038a because electronic device 600 has not detected any potential health issues for the user based on entered menstrual cycle information) (in some embodiments, re-displaying the cycle tracking user interface includes forgoing display of the first indicator). In some embodiments, in accordance with a determination that the second updated menstrual cycle information satisfies the first set of criteria, re-displaying the cycle tracking user interface includes displaying (within the cycle tracking user interface) the first indicator. Excluding the first indicator and/or forgoing display of the first indicator in accordance with a determination that the second updated menstrual cycle information does not satisfy the first set of criteria improves the user-device interface by ensuring that inaccurate, irrelevant, and/or faulty information is not presented to the user.

In some embodiments, subsequent to displaying the push notification (e.g., 1052), the computer system (e.g., 600) receives, via the one or more input devices, one or more confirmation inputs (e.g., 1056) (e.g., one or more touch inputs, one or more non-touch inputs, and/or one or more gestures) confirming the menstrual cycle information for the user (e.g., one or more user inputs indicating that the user confirms the accuracy of the menstrual cycle information for the user and/or indicating that the user confirms the accuracy of menstrual cycle history information for the user). In response to receiving the one or more confirmation inputs: in accordance with a determination that the user of the computer system has elected to share menstrual cycle information with a second user different from the user of the computer system, the computer system causes an external device (e.g., 1070) (e.g., a wearable device, a smart watch, a smart phone, a tablet, and/or a computer system controlling an external display) (e.g., an external device corresponding to an external user different from the user of the computer system) separate from the computer system and corresponding to the second user to display (e.g., via one or more display generation components in communication with the external device) a first visual indication (e.g., 1076a and/or 1082e) indicating that the computer system (e.g., 600) has identified a potential health issue for the user. In some embodiments, subsequent to displaying the push notification, and prior to receiving the one or more confirmation inputs, the computer system forgoes causing and/or does not cause the external device to display the first visual indication. Automatically causing the external device to display the first visual indication in response to receiving the one or more confirmation inputs allows a user to share health information with fewer user inputs, thereby reducing the number of user inputs required to perform an operation.

In some embodiments, the computer system (e.g., 600) displays, via the display generation component (e.g., 602), a cycle history user interface (e.g., 848 and/or 854) (e.g., a user interface that displays historical menstrual cycle information for a user), wherein the cycle history user interface includes one or more health indications indicative of one or more previously identified health issues that were identified by the computer system and confirmed by the user (e.g., in some embodiments, cycle history user interface 848 and/or user interface 854 displays an indication of one or more confirmed potential health issues) (e.g., confirmed by the user based on one or more user inputs confirming the accuracy of underlying menstrual cycle information that was used by the computer system to identify a potential health issue), including: a first health indication indicative of a first previously identified health issue that was identified by the computer system and confirmed by the user. In some embodiments, the cycle history user interface does not identify one or more previously identified health issues that were identified by the computer system but were not confirmed by the user (e.g., the user did not confirm the accuracy of underlying menstrual cycle information that was used by the computer system to identify a potential health issue). In some embodiments, the cycle history user interface includes a second health indication indicative of a second previously identified health issue that was identified by the computer system and confirm by the user, wherein the second previously identified health issue is different from the first previously identified health issue and the second health indication is different from the first health indication. Displaying a cycle history user interface that displays a history of confirmed health issues provides the user with visual feedback about the state of the system (e.g., that the system has identified one or more potential health issues for the user), thereby providing improved visual feedback to the user.

In some embodiments, in accordance with a determination that the first previously identified health issue occurred within a threshold duration of time (e.g., within the last 6 months, within the last 12 months, or within the last 18 months) (e.g., the first previously identified health issue was identified by the computer system within the threshold duration of time, the first previously identified health issue was identified by the computer system based on menstrual cycle information that occurred (e.g., was experienced by the user) within the threshold duration of time, and/or the first previously identified health issue was confirmed by the user within the threshold duration of time), displaying the cycle history user interface (e.g., 848 and/or 854) further includes displaying a first export option corresponding to the first previously identified health issue and that is selectable to initiate a process for exporting (e.g., printing, saving, sending, and/or transmitting) a file (e.g., a document file, a PDF file, and/or a graphics file) containing menstrual cycle history information corresponding to the user (e.g., a 12-month menstrual cycle history for the user) (e.g., in some embodiments, cycle history user interface 848 and/or user interface 854 includes a selectable option that is selectable to export menstrual cycle information for one or more confirmed potential health issues that occurred within the threshold period of time); and in accordance with a determination that the first previously identified health issue did not occur within the threshold duration of time, the cycle history user interface does not include the first export option corresponding to the first previously identified health issue. Displaying an export option only if the previously identified health issue occurred within the threshold duration of time improves the user-device interface by helping the user to provide correct inputs, and preventing the user from exporting unimportant, outdated, and/or irrelevant information.

Note that details of the processes described above with respect to method 1100 (e.g., FIG. 11) are also applicable in an analogous manner to the methods described above. For example, method 700, method 900, and/or method 950 optionally include one or more of the characteristics of the various methods described above with reference to method 1100. For example, the body temperature information that is collected in method 700 can be used to generate health-based notifications and/or predictions in methods 900, 950, and/or 1100. For brevity, these details are not repeated below.

The foregoing description, for purpose of explanation, has been described with reference to specific embodiments. However, the illustrative discussions above are not intended to be exhaustive or to limit the invention to the precise forms disclosed. Many modifications and variations are possible in view of the above teachings. The embodiments were chosen and described in order to best explain the principles of the techniques and their practical applications. Others skilled in the art are thereby enabled to best utilize the techniques and various embodiments with various modifications as are suited to the particular use contemplated.

Although the disclosure and examples have been fully described with reference to the accompanying drawings, it is to be noted that various changes and modifications will become apparent to those skilled in the art. Such changes and modifications are to be understood as being included within the scope of the disclosure and examples as defined by the claims.

As described above, one aspect of the present technology is the gathering and use of data available from various sources to improve the delivery to users of health-related notifications and/or information or any other content that may be of interest to them. The present disclosure contemplates that in some instances, this gathered data may include personal information data that uniquely identifies or can be used to contact or locate a specific person. Such personal information data can include demographic data, location-based data, telephone numbers, email addresses, social network IDs, home addresses, data or records relating to a user's health or level of fitness (e.g., vital signs measurements, medication information, exercise information), date of birth, or any other identifying or personal information.

The present disclosure recognizes that the use of such personal information data, in the present technology, can be used to the benefit of users. For example, the personal information data can be used to deliver health-related notifications and/or information that is of interest to the user. Accordingly, use of such personal information data enables users to have calculated control of the delivered content. Further, other uses for personal information data that benefit the user are also contemplated by the present disclosure. For instance, health and fitness data may be used to provide insights into a user's general wellness, or may be used as positive feedback to individuals using technology to pursue wellness goals.

The present disclosure contemplates that the entities responsible for the collection, analysis, disclosure, transfer, storage, or other use of such personal information data will comply with well-established privacy policies and/or privacy practices. In particular, such entities should implement and consistently use privacy policies and practices that are generally recognized as meeting or exceeding industry or governmental requirements for maintaining personal information data private and secure. Such policies should be easily accessible by users, and should be updated as the collection and/or use of data changes. Personal information from users should be collected for legitimate and reasonable uses of the entity and not shared or sold outside of those legitimate uses. Further, such collection/sharing should occur after receiving the informed consent of the users. Additionally, such entities should consider taking any needed steps for safeguarding and securing access to such personal information data and ensuring that others with access to the personal information data adhere to their privacy policies and procedures. Further, such entities can subject themselves to evaluation by third parties to certify their adherence to widely accepted privacy policies and practices. In addition, policies and practices should be adapted for the particular types of personal information data being collected and/or accessed and adapted to applicable laws and standards, including jurisdiction-specific considerations. For instance, in the US, collection of or access to certain health data may be governed by federal and/or state laws, such as the Health Insurance Portability and Accountability Act (HIPAA); whereas health data in other countries may be subject to other regulations and policies and should be handled accordingly. Hence different privacy practices should be maintained for different personal data types in each country.

Despite the foregoing, the present disclosure also contemplates embodiments in which users selectively block the use of, or access to, personal information data. That is, the present disclosure contemplates that hardware and/or software elements can be provided to prevent or block access to such personal information data. For example, in the case of health-related information, the present technology can be configured to allow users to select to “opt in” or “opt out” of participation in the collection of personal information data during registration for services or anytime thereafter. In another example, users can select not to receive health-related notifications, predications, and/or information. In yet another example, users can select to limit the length of time health data is maintained or entirely prohibit health-related notifications and/or predictions. In addition to providing “opt in” and “opt out” options, the present disclosure contemplates providing notifications relating to the access or use of personal information. For instance, a user may be notified upon downloading an app that their personal information data will be accessed and then reminded again just before personal information data is accessed by the app.

Moreover, it is the intent of the present disclosure that personal information data should be managed and handled in a way to minimize risks of unintentional or unauthorized access or use. Risk can be minimized by limiting the collection of data and deleting data once it is no longer needed. In addition, and when applicable, including in certain health related applications, data de-identification can be used to protect a user's privacy. De-identification may be facilitated, when appropriate, by removing specific identifiers (e.g., date of birth, etc.), controlling the amount or specificity of data stored (e.g., collecting location data a city level rather than at an address level), controlling how data is stored (e.g., aggregating data across users), and/or other methods.

Therefore, although the present disclosure broadly covers use of personal information data to implement one or more various disclosed embodiments, the present disclosure also contemplates that the various embodiments can also be implemented without the need for accessing such personal information data. That is, the various embodiments of the present technology are not rendered inoperable due to the lack of all or a portion of such personal information data. For example, content can be selected and delivered to users by inferring preferences based on non-personal information data or a bare minimum amount of personal information, such as the content being requested by the device associated with a user, other non-personal information available to the content delivery services and/or health information services, or publicly available information.

1-100. (canceled)

101. A computer system configured to communicate with a display generation component, one or more input devices, and a temperature sensor, comprising:
one or more processors; and
memory storing one or more programs configured to be executed by the one or more processors, the one or more programs including instructions for:
receiving, via the one or more input devices, an input corresponding to a request to enter a sleep tracking mode;
in response to the input, entering the sleep tracking mode;
during the sleep tracking mode, causing a first set of user body temperature information to be collected via the temperature sensor;
receiving notification data of a first type;
in response to receiving the notification data of the first type:
in accordance with a determination that the computer system is not in the sleep tracking mode, outputting a first notification corresponding to the notification data of the first type; and
in accordance with a determination that the computer system is currently in the sleep tracking mode, forgoing outputting the first notification corresponding to the notification data of the first type; and
after causing the first set of user body temperature information to be collected, displaying, via display generation component, a body temperature user interface that includes representations of one or more sets of user body temperature information including the first set of user body temperature information.

102. The computer system of claim 101, wherein the one or more programs further include instructions for:
receiving, via the one or more input devices, a second input corresponding to a request to enter a first silent mode different from the sleep tracking mode;
in response to receiving the second input, entering the first silent mode without initiating a process to collect body temperature information;
receiving second notification data of the first type; and
in response to receiving the second notification data of the first type:
in accordance with a determination that the computer system is not in the first silent mode and that the computer system is not in the sleep tracking mode, outputting a second notification corresponding to the second notification data of the first type; and
in accordance with a determination that the computer system is currently in the first silent mode, forgoing outputting a notification corresponding to the second notification data of the first type.

103. The computer system of claim 101, wherein the one or more programs further include instructions for:
receiving, via the one or more input devices, a third input corresponding to a request to define a sleep schedule, wherein the sleep schedule includes, for at least a first day, a first sleep start time and a first sleep end time;
subsequent to receiving the third input:
in accordance with a determination that a current time corresponds to the first sleep start time, entering the sleep tracking mode; and
in accordance with a determination that the current time does not correspond to the first sleep start time, forgoing entering the sleep tracking mode.

104. The computer system of claim 101, wherein causing the first set of user body temperature information to be collected includes:
causing a first external device separate from the computer system to collect the first set of user body temperature information; and
receiving the first set of user body temperature information from the first external device.

105. The computer system of claim 101, wherein the one or more programs further include instructions for:
prior to receiving the input, displaying, via the display generation component, the body temperature user interface, including:
in accordance with a determination that a threshold amount of body temperature information has not been collected, displaying an indication that the threshold amount of body temperature information has not been collected; and
in accordance with a determination that the threshold amount of body temperature information has been collected, displaying representations of one or more sets of user body temperature information without displaying the indication that the threshold amount of body temperature information has not been collected.

106. The computer system of claim 101, wherein the one or more programs further include instructions for:
subsequent to displaying the body temperature user interface that includes the representations of one or more sets of user body temperature information, displaying a second instance of the body temperature user interface, wherein:
in accordance with a determination that a second set of criteria are satisfied, the second instance of the body temperature user interface includes representations of a second set of user body temperature information; and
in accordance with a determination that the second set of criteria are not satisfied, the second instance of the body temperature user interface does not include the representations of the second set of user body temperature information and includes a second indication indicating that the second set of criteria are not satisfied.

107. The computer system of claim 101, wherein the one or more programs further include instructions for:
while displaying the body temperature user interface, receiving, via the one or more input devices, a selection input; and
in response to receiving the selection input:
in accordance with a determination that the selection input corresponds to selection of a first timeframe option corresponding to a first timeframe, displaying, within the body temperature user interface, representations of one or more sets of user body temperature information corresponding to the first timeframe; and
in accordance with a determination that the selection input corresponds to selection of a second timeframe option different from the first timeframe option, wherein the second timeframe option corresponds to a second timeframe different from the first timeframe, displaying, within the body temperature user interface, representations of one or more sets of user body temperature information corresponding to the second timeframe.

108. The computer system of claim 101, wherein the representations of one or more sets of user body temperature information includes one or more relative temperature measurements.

109. The computer system of claim 101, wherein the one or more programs further include instructions for:
displaying, via the display generation component, a first details user interface corresponding to the first set of user body temperature information, wherein the first details user interface includes information about the first set of user body temperature information that is not displayed in the body temperature user interface.

110. The computer system of claim 109, wherein the first details user interface includes:
a start time corresponding to the first set of user body temperature information; and
an end time corresponding to the first set of user body temperature information.

111. The computer system of claim 109, wherein:
the body temperature user interface includes relative body temperature information corresponding to the first set of user body temperature information; and
the first details user interface includes one or more absolute body temperature measurements corresponding to the first set of user body temperature information.

112. The computer system of claim 109, wherein the first details user interface includes external device information corresponding to the first set of user body temperature information.

113. The computer system of claim 101, wherein the one or more programs further include instructions for:
receiving first notification data corresponding to one or more messages received from a first external user and/or one or more messages received from a first application;
in response to receiving the first notification data:
in accordance with a determination that the computer system is not in the sleep tracking mode, outputting a second notification corresponding to the first notification data; and
in accordance with a determination that the computer system is currently in the sleep tracking mode, and that the first notification data comprises notification data of a second type, forgoing outputting the second notification corresponding to the first notification data.

114. The computer system of claim 113, wherein the one or more programs further include instructions for:
in response to receiving the first notification data:
in accordance with a determination that the computer system is currently in the sleep tracking mode, and that the first notification data comprises notification data of a third type different from the second type, outputting the second notification corresponding to the first notification data.

115. The computer system of claim 101, wherein the one or more programs further include instructions for:
during the sleep tracking mode:
in accordance with a determination that the computer system is in the sleep tracking mode, causing a second external device different from the computer system to display an indication that the computer system has suppressed notifications.

116. The computer system of claim 101, wherein:
the representations of one or more sets of user body temperature information includes:
a representation of a third set of user body temperature information collected by a second external device separate from the computer system; and
a representation of a fourth set of user body temperature information different from the second set of user body temperature information and collected by a third external device different from the second external device and the computer system.

117. A non-transitory computer-readable storage medium storing one or more programs configured to be executed by one or more processors of a computer system that is in communication with a display generation component, one or more input devices, and a temperature sensor, the one or more programs including instructions for:
receiving, via the one or more input devices, an input corresponding to a request to enter a sleep tracking mode;
in response to the input, entering the sleep tracking mode;
during the sleep tracking mode, causing a first set of user body temperature information to be collected via the temperature sensor;
receiving notification data of a first type;
in response to receiving the notification data of the first type:
in accordance with a determination that the computer system is not in the sleep tracking mode, outputting a first notification corresponding to the notification data of the first type; and
in accordance with a determination that the computer system is currently in the sleep tracking mode, forgoing outputting the first notification corresponding to the notification data of the first type; and
after causing the first set of user body temperature information to be collected, displaying, via display generation component, a body temperature user interface that includes representations of one or more sets of user body temperature information including the first set of user body temperature information.

118. A method, comprising:
at a computer system that is in communication with a display generation component, one or more input devices, and a temperature sensor:
receiving, via the one or more input devices, an input corresponding to a request to enter a sleep tracking mode;
in response to the input, entering the sleep tracking mode;
during the sleep tracking mode, causing a first set of user body temperature information to be collected via the temperature sensor;
receiving notification data of a first type;
in response to receiving the notification data of the first type:
in accordance with a determination that the computer system is not in the sleep tracking mode, outputting a first notification corresponding to the notification data of the first type; and
in accordance with a determination that the computer system is currently in the sleep tracking mode, forgoing outputting the first notification corresponding to the notification data of the first type; and
after causing the first set of user body temperature information to be collected, displaying, via display generation component, a body temperature user interface that includes representations of one or more sets of user body temperature information including the first set of user body temperature information.
